SEQUENCE LISTING (349 sequences)

SEQ ID NO: 1 - protein, 10 aa, Artificial Sequence
  Description of Artificial Sequence Synthetic peptide
GSTFSMAAWA

SEQ ID NO: 2 - protein, 16 aa, Artificial Sequence
  Description of Artificial Sequence Synthetic peptide
SITAGGHTNYADSVEG

SEQ ID NO: 3 - protein, 11 aa, Artificial Sequence
  Description of Artificial Sequence Synthetic peptide
FADVYGSDRPS

SEQ ID NO: 4 - protein, 10 aa, Artificial Sequence
  Description of Artificial Sequence Synthetic peptide
GSTFSINAIA

SEQ ID NO: 5 - protein, 16 aa, Artificial Sequence
  Description of Artificial Sequence Synthetic peptide
SITAGGHTNYADSVEG

SEQ ID NO: 6 - protein, 11 aa, Artificial Sequence
  Description of Artificial Sequence Synthetic peptide
FADVYGSDRPS

SEQ ID NO: 7 - protein, 10 aa, Artificial Sequence
  Description of Artificial Sequence Synthetic peptide
GRTWGMVVMG

SEQ ID NO: 8 - protein, 17 aa, Artificial Sequence
  Description of Artificial Sequence Synthetic peptide
AINWNGGSTQYGNSVKG

SEQ ID NO: 9 - protein, 12 aa, Artificial Sequence
  Description of Artificial Sequence Synthetic peptide
NAVEFGWGGYDY

SEQ ID NO: 10 - protein, 8 aa, Artificial Sequence
  Description of Artificial Sequence Synthetic peptide
GFKINAMA

SEQ ID NO: 11 - protein, 19 aa, Artificial Sequence
  Description of Artificial Sequence Synthetic peptide
LVASITAGGHTNYADSVEG

SEQ ID NO: 12 - protein, 11 aa, Artificial Sequence
  Description of Artificial Sequence Synthetic peptide
FADVYGSDRPS

SEQ ID NO: 13 - protein, 10 aa, Artificial Sequence
  Description of Artificial Sequence Synthetic peptide
GGLFSLSAMG

SEQ ID NO: 14 - protein, 16 aa, Artificial Sequence
  Description of Artificial Sequence Synthetic peptide
TITLGGSTTYGDSVKG

SEQ ID NO: 15 - protein, 18 aa, Artificial Sequence
  Description of Artificial Sequence Synthetic peptide
NADASRWTGDRWTAYDRV

SEQ ID NO: 16 - protein, 10 aa, Artificial Sequence
  Description of Artificial Sequence Synthetic peptide
ESIDSLAAMG

SEQ ID NO: 17 - protein, 16 aa, Artificial Sequence
  Description of Artificial Sequence Synthetic peptide
TISKEGHINYADSVKG

SEQ ID NO: 18 - protein, 11 aa, Artificial Sequence
  Description of Artificial Sequence Synthetic peptide
FADVYGSDRPS

SEQ ID NO: 19 - protein, 10 aa, Artificial Sequence
  Description of Artificial Sequence Synthetic peptide
GGLFSLSAMG

SEQ ID NO: 20 - protein, 16 aa, Artificial Sequence
  Description of Artificial Sequence Synthetic peptide
TITLGGSTTYGDSVKG

SEQ ID NO: 21 - protein, 18 aa, Artificial Sequence
  Description of Artificial Sequence Synthetic peptide
NADASRWTGDRWTAYDRV

SEQ ID NO: 22 - protein, 10 aa, Artificial Sequence
  Description of Artificial Sequence Synthetic peptide
GFTFSSYLMY

SEQ ID NO: 23 - protein, 17 aa, Artificial Sequence
  Description of Artificial Sequence Synthetic peptide
AIQPGGGSTYYADSVKG

SEQ ID NO: 24 - protein, 14 aa, Artificial Sequence
  Description of Artificial Sequence Synthetic peptide
NAEWGYYSGAYRSY

SEQ ID NO: 25 - protein, 10 aa, Artificial Sequence
  Description of Artificial Sequence Synthetic peptide
GRDSSRDAMA

SEQ ID NO: 26 - protein, 16 aa, Artificial Sequence
  Description of Artificial Sequence Synthetic peptide
SITAGGHTNYADSVEG

SEQ ID NO: 27 - protein, 11 aa, Artificial Sequence
  Description of Artificial Sequence Synthetic peptide
FADVYGSDRPS

SEQ ID NO: 28 - protein, 10 aa, Artificial Sequence
  Description of Artificial Sequence Synthetic peptide
GFTLPYYAIV

SEQ ID NO: 29 - protein, 16 aa, Artificial Sequence
  Description of Artificial Sequence Synthetic peptide
SITAGGHTNYADSVEG

SEQ ID NO: 30 - protein, 11 aa, Artificial Sequence
  Description of Artificial Sequence Synthetic peptide
FADVYGSDRPS

SEQ ID NO: 31 - protein, 11 aa, Artificial Sequence
  Description of Artificial Sequence Synthetic peptide
GRTTPNIYAMS

SEQ ID NO: 32 - protein, 17 aa, Artificial Sequence
  Description of Artificial Sequence Synthetic peptide
TINSGGFSPAYADSVKG

SEQ ID NO: 33 - protein, 11 aa, Artificial Sequence
  Description of Artificial Sequence Synthetic peptide
ARHMGYEAMDY

SEQ ID NO: 34 - protein, 10 aa, Artificial Sequence
  Description of Artificial Sequence Synthetic peptide
GRIFSINAMG

SEQ ID NO: 35 - protein, 16 aa, Artificial Sequence
  Description of Artificial Sequence Synthetic peptide
AIAMTGSTSYGDSVKG

SEQ ID NO: 36 - protein, 19 aa, Artificial Sequence
  Description of Artificial Sequence Synthetic peptide
YATVFLSPPDDEDEGLLEL

SEQ ID NO: 37 - protein, 10 aa, Artificial Sequence
  Description of Artificial Sequence Synthetic peptide
GSIFSINVRA

SEQ ID NO: 38 - protein, 16 aa, Artificial Sequence
  Description of Artificial Sequence Synthetic peptide
SITAGGHTNYADSVEG

SEQ ID NO: 39 - protein, 11 aa, Artificial Sequence
  Description of Artificial Sequence Synthetic peptide
FADVYGSDRPS

SEQ ID NO: 40 - protein, 10 aa, Artificial Sequence
  Description of Artificial Sequence Synthetic peptide
GNTFSINAMN

SEQ ID NO: 41 - protein, 16 aa, Artificial Sequence
  Description of Artificial Sequence Synthetic peptide
TISSDGTANYAGSVKG

SEQ ID NO: 42 - protein, 19 aa, Artificial Sequence
  Description of Artificial Sequence Synthetic peptide
YVVAAVFKEDYDSPPFHDY

SEQ ID NO: 43 - protein, 10 aa, Artificial Sequence
  Description of Artificial Sequence Synthetic peptide
SSISSINVMA

SEQ ID NO: 44 - protein, 16 aa, Artificial Sequence
  Description of Artificial Sequence Synthetic peptide
TISKEGHINYADSVKG

SEQ ID NO: 45 - protein, 11 aa, Artificial Sequence
  Description of Artificial Sequence Synthetic peptide
FADVYGSDRPS

SEQ ID NO: 46 - protein, 10 aa, Artificial Sequence
  Description of Artificial Sequence Synthetic peptide
GRTLSRYTMA

SEQ ID NO: 47 - protein, 17 aa, Artificial Sequence
  Description of Artificial Sequence Synthetic peptide
AINWSGDRVSYTDSVKG

SEQ ID NO: 48 - protein, 16 aa, Artificial Sequence
  Description of Artificial Sequence Synthetic peptide
YAIPYYSDSEENLYDY

SEQ ID NO: 49 - protein, 11 aa, Artificial Sequence
  Description of Artificial Sequence Synthetic peptide
SESRFHYNTMA

SEQ ID NO: 50 - protein, 16 aa, Artificial Sequence
  Description of Artificial Sequence Synthetic peptide
SITAGGHTNYADSVEG

SEQ ID NO: 51 - protein, 11 aa, Artificial Sequence
  Description of Artificial Sequence Synthetic peptide
FADVYGSDRPS

SEQ ID NO: 52 - protein, 10 aa, Artificial Sequence
  Description of Artificial Sequence Synthetic peptide
GSGRSINVMG

SEQ ID NO: 53 - protein, 16 aa, Artificial Sequence
  Description of Artificial Sequence Synthetic peptide
LIRSGGSTDYADSVKD

SEQ ID NO: 54 - protein, 11 aa, Artificial Sequence
  Description of Artificial Sequence Synthetic peptide
NAERYSRALRP

SEQ ID NO: 55 - protein, 12 aa, Artificial Sequence
  Description of Artificial Sequence Synthetic peptide
GRTFSFSMYNMA

SEQ ID NO: 56 - protein, 17 aa, Artificial Sequence
  Description of Artificial Sequence Synthetic peptide
AISWSGSSADYADFVKG

SEQ ID NO: 57 - protein, 10 aa, Artificial Sequence
  Description of Artificial Sequence Synthetic peptide
YAVRIDGMDY

SEQ ID NO: 58 - protein, 10 aa, Artificial Sequence
  Description of Artificial Sequence Synthetic peptide
EATFSKYVMG

SEQ ID NO: 59 - protein, 17 aa, Artificial Sequence
  Description of Artificial Sequence Synthetic peptide
TIDWKGSSTHYADSVEG

SEQ ID NO: 60 - protein, 18 aa, Artificial Sequence
  Description of Artificial Sequence Synthetic peptide
AADLALSMRYYHAPNYEY

SEQ ID NO: 61 - protein, 10 aa, Artificial Sequence
  Description of Artificial Sequence Synthetic peptide
GFTFSTYWMH

SEQ ID NO: 62 - protein, 16 aa, Artificial Sequence
  Description of Artificial Sequence Synthetic peptide
AINSDSSALYADSVKG

SEQ ID NO: 63 - protein, 20 aa, Artificial Sequence
  Description of Artificial Sequence Synthetic peptide
SASIGVRPYSGSDLLRRLEV

SEQ ID NO: 64 - protein, 10 aa, Artificial Sequence
  Description of Artificial Sequence Synthetic peptide
EATFSRYVMG

SEQ ID NO: 65 - protein, 17 aa, Artificial Sequence
  Description of Artificial Sequence Synthetic peptide
AIDWKGSSTHYADSVEG

SEQ ID NO: 66 - protein, 18 aa, Artificial Sequence
  Description of Artificial Sequence Synthetic peptide
AADLALSMRYYHAPNYEY

SEQ ID NO: 67 - protein, 10 aa, Artificial Sequence
  Description of Artificial Sequence Synthetic peptide
GRSFSPYTMG

SEQ ID NO: 68 - protein, 17 aa, Artificial Sequence
  Description of Artificial Sequence Synthetic peptide
ASSASGGSTTYASSGKD

SEQ ID NO: 69 - protein, 10 aa, Artificial Sequence
  Description of Artificial Sequence Synthetic peptide
YVNIWNGGDY

SEQ ID NO: 70 - protein, 10 aa, Artificial Sequence
  Description of Artificial Sequence Synthetic peptide
TSIFNMNAMN

SEQ ID NO: 71 - protein, 17 aa, Artificial Sequence
  Description of Artificial Sequence Synthetic peptide
TINSGGTYTSYADSVKG

SEQ ID NO: 72 - protein, 15 aa, Artificial Sequence
  Description of Artificial Sequence Synthetic peptide
NAVVNTILDFLPKNY

SEQ ID NO: 73 - protein, 10 aa, Artificial Sequence
  Description of Artificial Sequence Synthetic peptide
GGSSDQYTIG

SEQ ID NO: 74 - protein, 16 aa, Artificial Sequence
  Description of Artificial Sequence Synthetic peptide
SSRSGGSLAYNDSIKD

SEQ ID NO: 75 - protein, 17 aa, Artificial Sequence
  Description of Artificial Sequence Synthetic peptide
AAGFGPAVPVTPDEYTY

SEQ ID NO: 76 - protein, 10 aa, Artificial Sequence
  Description of Artificial Sequence Synthetic peptide
GNTSSIVDME

SEQ ID NO: 77 - protein, 16 aa, Artificial Sequence
  Description of Artificial Sequence Synthetic peptide
FIRRAGTTVYASSVKG

SEQ ID NO: 78 - protein, 10 aa, Artificial Sequence
  Description of Artificial Sequence Synthetic peptide
NANDWGGREY

SEQ ID NO: 79 - protein, 10 aa, Artificial Sequence
  Description of Artificial Sequence Synthetic peptide
GSIFSYSTMG

SEQ ID NO: 80 - protein, 16 aa, Artificial Sequence
  Description of Artificial Sequence Synthetic peptide
SIPRAGTRTYLDSVKG

SEQ ID NO: 81 - protein, 5 aa, Artificial Sequence
  Description of Artificial Sequence Synthetic peptide
RGDNY

SEQ ID NO: 82 - protein, 10 aa, Artificial Sequence
  Description of Artificial Sequence Synthetic peptide
GFTFSRYWMY

SEQ ID NO: 83 - protein, 17 aa, Artificial Sequence
  Description of Artificial Sequence Synthetic peptide
TINTGGGSTYYADSVKG

SEQ ID NO: 84 - protein, 13 aa, Artificial Sequence
  Description of Artificial Sequence Synthetic peptide
AKGHSNYEDALDA

SEQ ID NO: 85 - protein, 10 aa, Artificial Sequence
  Description of Artificial Sequence Synthetic peptide
GFTFDNYFLS

SEQ ID NO: 86 - protein, 17 aa, Artificial Sequence
  Description of Artificial Sequence Synthetic peptide
GTYGAIDATYYEDSVKG

SEQ ID NO: 87 - protein, 15 aa, Artificial Sequence
  Description of Artificial Sequence Synthetic peptide
AAGRLVTSTTSSYDY

SEQ ID NO: 88 - protein, 10 aa, Artificial Sequence
  Description of Artificial Sequence Synthetic peptide
GRTVDDDNMG

SEQ ID NO: 89 - protein, 17 aa, Artificial Sequence
  Description of Artificial Sequence Synthetic peptide
TINSRGWEIYYSDSVKG

SEQ ID NO: 90 - protein, 17 aa, Artificial Sequence
  Description of Artificial Sequence Synthetic peptide
AAGPYGTGWNLDNQYTY

SEQ ID NO: 91 - protein, 10 aa, Artificial Sequence
  Description of Artificial Sequence Synthetic peptide
GGKSDQYTIA

SEQ ID NO: 92 - protein, 16 aa, Artificial Sequence
  Description of Artificial Sequence Synthetic peptide
SSRSGGSLTYIDSIKD

SEQ ID NO: 93 - protein, 17 aa, Artificial Sequence
  Description of Artificial Sequence Synthetic peptide
AAGYGPAVPVTPDEYTY

SEQ ID NO: 94 - protein, 10 aa, Artificial Sequence
  Description of Artificial Sequence Synthetic peptide
GSIFGINVMA

SEQ ID NO: 95 - protein, 16 aa, Artificial Sequence
  Description of Artificial Sequence Synthetic peptide
TISAGGIINYADFVEG

SEQ ID NO: 96 - protein, 11 aa, Artificial Sequence
  Description of Artificial Sequence Synthetic peptide
FADVAGSDRPS

SEQ ID NO: 97 - protein, 10 aa, Artificial Sequence
  Description of Artificial Sequence Synthetic peptide
GRTLSNYNVG

SEQ ID NO: 98 - protein, 17 aa, Artificial Sequence
  Description of Artificial Sequence Synthetic peptide
AISQSGDMTSYGDSVKG

SEQ ID NO: 99 - protein, 16 aa, Artificial Sequence
  Description of Artificial Sequence Synthetic peptide
AKLDAEGYGGDRYLEV

SEQ ID NO: 100 - protein, 10 aa, Artificial Sequence
  Description of Artificial Sequence Synthetic peptide
GLTFDDVPIG

SEQ ID NO: 101 - protein, 17 aa, Artificial Sequence
  Description of Artificial Sequence Synthetic peptide
RIEVSDDSTYYADSVKG

SEQ ID NO: 102 - protein, 15 aa, Artificial Sequence
  Description of Artificial Sequence Synthetic peptide
AADSSYYGLGYDYNT

SEQ ID NO: 103 - protein, 10 aa, Artificial Sequence
  Description of Artificial Sequence Synthetic peptide
RSIFSIAMMG

SEQ ID NO: 104 - protein, 16 aa, Artificial Sequence
  Description of Artificial Sequence Synthetic peptide
TITTDGNTNYADFVKG

SEQ ID NO: 105 - protein, 8 aa, Artificial Sequence
  Description of Artificial Sequence Synthetic peptide
IARKYGTY

SEQ ID NO: 106 - protein, 10 aa, Artificial Sequence
  Description of Artificial Sequence Synthetic peptide
TRPSNLIGMG

SEQ ID NO: 107 - protein, 17 aa, Artificial Sequence
  Description of Artificial Sequence Synthetic peptide
AIRWSGGTAYYADSVKG

SEQ ID NO: 108 - protein, 9 aa, Artificial Sequence
  Description of Artificial Sequence Synthetic peptide
KAVDGGLGY

SEQ ID NO: 109 - protein, 11 aa, Artificial Sequence
  Description of Artificial Sequence Synthetic peptide
GSIASALNAMG

SEQ ID NO: 110 - protein, 16 aa, Artificial Sequence
  Description of Artificial Sequence Synthetic peptide
SITSGGTTTYAKSVKG

SEQ ID NO: 111 - protein, 18 aa, Artificial Sequence
  Description of Artificial Sequence Synthetic peptide
AASHFDYRDYAFTSEYDY

SEQ ID NO: 112 - protein, 10 aa, Artificial Sequence
  Description of Artificial Sequence Synthetic peptide
GDTLDKYHIG

SEQ ID NO: 113 - protein, 17 aa, Artificial Sequence
  Description of Artificial Sequence Synthetic peptide
AIKWSDGDTSYARSVKG

SEQ ID NO: 114 - protein, 10 aa, Artificial Sequence
  Description of Artificial Sequence Synthetic peptide
YVVTVRTGDY

SEQ ID NO: 115 - protein, 10 aa, Artificial Sequence
  Description of Artificial Sequence Synthetic peptide
GTTSRINTMA

SEQ ID NO: 116 - protein, 17 aa, Artificial Sequence
  Description of Artificial Sequence Synthetic peptide
AIVINGGTTSYGASVKG

SEQ ID NO: 117 - protein, 10 aa, Artificial Sequence
  Description of Artificial Sequence Synthetic peptide
HAISVPDGDY

SEQ ID NO: 118 - protein, 10 aa, Artificial Sequence
  Description of Artificial Sequence Synthetic peptide
GSIFSYSTMG

SEQ ID NO: 119 - protein, 16 aa, Artificial Sequence
  Description of Artificial Sequence Synthetic peptide
SIPRAGTRTYLDSVKG

SEQ ID NO: 120 - protein, 5 aa, Artificial Sequence
  Description of Artificial Sequence Synthetic peptide
RGDNY

SEQ ID NO: 121 - protein, 10 aa, Artificial Sequence
  Description of Artificial Sequence Synthetic peptide
GLTFSMYTMA

SEQ ID NO: 122 - protein, 17 aa, Artificial Sequence
  Description of Artificial Sequence Synthetic peptide
TINFDVVTTTYADSVKG

SEQ ID NO: 123 - protein, 15 aa, Artificial Sequence
  Description of Artificial Sequence Synthetic peptide
AVGDFYSRSQRQYTY

SEQ ID NO: 124 - protein, 10 aa, Artificial Sequence
  Description of Artificial Sequence Synthetic peptide
GPIVRFTAMG

SEQ ID NO: 125 - protein, 16 aa, Artificial Sequence
  Description of Artificial Sequence Synthetic peptide
TISKEGHINYADSVKG

SEQ ID NO: 126 - protein, 11 aa, Artificial Sequence
  Description of Artificial Sequence Synthetic peptide
FADVYGSDRPS

SEQ ID NO: 127 - protein, 10 aa, Artificial Sequence
  Description of Artificial Sequence Synthetic peptide
GPTFNPYAMA

SEQ ID NO: 128 - protein, 16 aa, Artificial Sequence
  Description of Artificial Sequence Synthetic peptide
SITAGGHTNYADSVEG

SEQ ID NO: 129 - protein, 11 aa, Artificial Sequence
  Description of Artificial Sequence Synthetic peptide
FADVYGSDRPS

SEQ ID NO: 130 - protein, 10 aa, Artificial Sequence
  Description of Artificial Sequence Synthetic peptide
GFTFSHYQMY

SEQ ID NO: 131 - protein, 17 aa, Artificial Sequence
  Description of Artificial Sequence Synthetic peptide
SIHTDGRNTYYQNSVKG

SEQ ID NO: 132 - protein, 16 aa, Artificial Sequence
  Description of Artificial Sequence Synthetic peptide
YAIPYYSDSEENLYDY

SEQ ID NO: 133 - protein, 10 aa, Artificial Sequence
  Description of Artificial Sequence Synthetic peptide
GSIFSINAMG

SEQ ID NO: 134 - protein, 15 aa, Artificial Sequence
  Description of Artificial Sequence Synthetic peptide
ITRPGTTNYAESVKG

SEQ ID NO: 135 - protein, 14 aa, Artificial Sequence
  Description of Artificial Sequence Synthetic peptide
NAQSGNYMLGFYDY

SEQ ID NO: 136 - protein, 10 aa, Artificial Sequence
  Description of Artificial Sequence Synthetic peptide
GRAVNRHIMG

SEQ ID NO: 137 - protein, 17 aa, Artificial Sequence
  Description of Artificial Sequence Synthetic peptide
SISWNSGRTYFADFVKG

SEQ ID NO: 138 - protein, 18 aa, Artificial Sequence
  Description of Artificial Sequence Synthetic peptide
RADYYVDYEDDRMGFFDA

SEQ ID NO: 139 - protein, 10 aa, Artificial Sequence
  Description of Artificial Sequence Synthetic peptide
GFTSDYYAIG

SEQ ID NO: 140 - protein, 20 aa, Artificial Sequence
  Description of Artificial Sequence Synthetic peptide
TVPWNDGIGGSKHYAEVVKG

SEQ ID NO: 141 - protein, 25 aa, Artificial Sequence
  Description of Artificial Sequence Synthetic peptide
AAAGPYRLYPEGHYPPDVNAYEYAY

SEQ ID NO: 142 - protein, 12 aa, Artificial Sequence
  Description of Artificial Sequence Synthetic peptide
GRSVVFSTMGMA

SEQ ID NO: 143 - protein, 17 aa, Artificial Sequence
  Description of Artificial Sequence Synthetic peptide
GVGVKGGSTTYRESVKG

SEQ ID NO: 144 - protein, 16 aa, Artificial Sequence
  Description of Artificial Sequence Synthetic peptide
YAIPYYSDSEENLYDY

SEQ ID NO: 145 - protein, 10 aa, Artificial Sequence
  Description of Artificial Sequence Synthetic peptide
GSTFNIETMS

SEQ ID NO: 146 - protein, 17 aa, Artificial Sequence
  Description of Artificial Sequence Synthetic peptide
SISVGGFPPYYTHSVKG

SEQ ID NO: 147 - protein, 11 aa, Artificial Sequence
  Description of Artificial Sequence Synthetic peptide
NAYSRAGSKYY

SEQ ID NO: 148 - protein, 10 aa, Artificial Sequence
  Description of Artificial Sequence Synthetic peptide
GFTLADYGIG

SEQ ID NO: 149 - protein, 17 aa, Artificial Sequence
  Description of Artificial Sequence Synthetic peptide
CMNKNDGNTFYAESVKG

SEQ ID NO: 150 - protein, 17 aa, Artificial Sequence
  Description of Artificial Sequence Synthetic peptide
AADILGYDCSGSASMDH

SEQ ID NO: 151 - protein, 10 aa, Artificial Sequence
  Description of Artificial Sequence Synthetic peptide
GFDFSSSRMY

SEQ ID NO: 152 - protein, 17 aa, Artificial Sequence
  Description of Artificial Sequence Synthetic peptide
SINAGGNTRDYSGSVKG

SEQ ID NO: 153 - protein, 15 aa, Artificial Sequence
  Description of Artificial Sequence Synthetic peptide
YGELKSASRFRVDNY

SEQ ID NO: 154 - protein, 10 aa, Artificial Sequence
  Description of Artificial Sequence Synthetic peptide
GFAAEGYAIT

SEQ ID NO: 155 - protein, 16 aa, Artificial Sequence
  Description of Artificial Sequence Synthetic peptide
HIRDGGITNYTDSVKG

SEQ ID NO: 156 - protein, 17 aa, Artificial Sequence
  Description of Artificial Sequence Synthetic peptide
NAGGITFSSGTFYPDPY

SEQ ID NO: 157 - protein, 10 aa, Artificial Sequence
  Description of Artificial Sequence Synthetic peptide
GFTFSNHAMS

SEQ ID NO: 158 - protein, 17 aa, Artificial Sequence
  Description of Artificial Sequence Synthetic peptide
FVSNGGRNTYYADSVKG

SEQ ID NO: 159 - protein, 17 aa, Artificial Sequence
  Description of Artificial Sequence Synthetic peptide
AASTTVFTGTTPGAYNY

SEQ ID NO: 160 - protein, 10 aa, Artificial Sequence
  Description of Artificial Sequence Synthetic peptide
GRSSSRYTMA

SEQ ID NO: 161 - protein, 17 aa, Artificial Sequence
  Description of Artificial Sequence Synthetic peptide
ALSQNGVTTYYADSVKG

SEQ ID NO: 162 - protein, 17 aa, Artificial Sequence
  Description of Artificial Sequence Synthetic peptide
GAAPSGALVQDINHYYY

SEQ ID NO: 163 - protein, 10 aa, Artificial Sequence
  Description of Artificial Sequence Synthetic peptide
GFIFSIRTMA

SEQ ID NO: 164 - protein, 16 aa, Artificial Sequence
  Description of Artificial Sequence Synthetic peptide
SITAGGHTNYADSVEG

SEQ ID NO: 165 - protein, 11 aa, Artificial Sequence
  Description of Artificial Sequence Synthetic peptide
FADVYGSDRPS

SEQ ID NO: 166 - protein, 10 aa, Artificial Sequence
  Description of Artificial Sequence Synthetic peptide
GSFYSINDMG

SEQ ID NO: 167 - protein, 16 aa, Artificial Sequence
  Description of Artificial Sequence Synthetic peptide
TITGRGRTNYKDSVKG

SEQ ID NO: 168 - protein, 17 aa, Artificial Sequence
  Description of Artificial Sequence Synthetic peptide
NVRAVVRILTHYRGSEV

SEQ ID NO: 169 - protein, 10 aa, Artificial Sequence
  Description of Artificial Sequence Synthetic peptide
GRTLSDYTMA

SEQ ID NO: 170 - protein, 17 aa, Artificial Sequence
  Description of Artificial Sequence Synthetic peptide
AIGWNGGNIYYADSVKG

SEQ ID NO: 171 - protein, 16 aa, Artificial Sequence
  Description of Artificial Sequence Synthetic peptide
YAIPYYSDSEENLYDY

SEQ ID NO: 172 - protein, 10 aa, Artificial Sequence
  Description of Artificial Sequence Synthetic peptide
GRPGITYTMG

SEQ ID NO: 173 - protein, 17 aa, Artificial Sequence
  Description of Artificial Sequence Synthetic peptide
GISGSGGSTAYADSVKG

SEQ ID NO: 174 - protein, 10 aa, Artificial Sequence
  Description of Artificial Sequence Synthetic peptide
LIVPRDGMDY

SEQ ID NO: 175 - protein, 10 aa, Artificial Sequence
  Description of Artificial Sequence Synthetic peptide
GFTLADYGIG

SEQ ID NO: 176 - protein, 17 aa, Artificial Sequence
  Description of Artificial Sequence Synthetic peptide
CMNKNDGNTFYAESVKG

SEQ ID NO: 177 - protein, 17 aa, Artificial Sequence
  Description of Artificial Sequence Synthetic peptide
AADILGYDCSGSASMDH

SEQ ID NO: 178 - protein, 10 aa, Artificial Sequence
  Description of Artificial Sequence Synthetic peptide
GFAFSDYAMN

SEQ ID NO: 179 - protein, 17 aa, Artificial Sequence
  Description of Artificial Sequence Synthetic peptide
AISWNGGSTYYAESIKG

SEQ ID NO: 180 - protein, 16 aa, Artificial Sequence
  Description of Artificial Sequence Synthetic peptide
AKSSTWHGRYYYGMDY

SEQ ID NO: 181 - protein, 10 aa, Artificial Sequence
  Description of Artificial Sequence Synthetic peptide
GLTFSMYSMG

SEQ ID NO: 182 - protein, 17 aa, Artificial Sequence
  Description of Artificial Sequence Synthetic peptide
SINPGGGSTVYADSVKG

SEQ ID NO: 183 - protein, 15 aa, Artificial Sequence
  Description of Artificial Sequence Synthetic peptide
GATIVELGRGYSTDY

SEQ ID NO: 184 - protein, 10 aa, Artificial Sequence
  Description of Artificial Sequence Synthetic peptide
GRSFGTYAVT

SEQ ID NO: 185 - protein, 17 aa, Artificial Sequence
  Description of Artificial Sequence Synthetic peptide
AISNSATTIQYTHSVKG

SEQ ID NO: 186 - protein, 11 aa, Artificial Sequence
  Description of Artificial Sequence Synthetic peptide
HAVAWDYGLDH

SEQ ID NO: 187 - protein, 10 aa, Artificial Sequence
  Description of Artificial Sequence Synthetic peptide
GFTSDYYVIG

SEQ ID NO: 188 - protein, 16 aa, Artificial Sequence
  Description of Artificial Sequence Synthetic peptide
TISKEGHINYADSVKG

SEQ ID NO: 189 - protein, 11 aa, Artificial Sequence
  Description of Artificial Sequence Synthetic peptide
FADVYGSDRPS

SEQ ID NO: 190 - protein, 10 aa, Artificial Sequence
  Description of Artificial Sequence Synthetic peptide
GSIFGINVMA

SEQ ID NO: 191 - protein, 17 aa, Artificial Sequence
  Description of Artificial Sequence Synthetic peptide
TIYNDASNTDYADSVKG

SEQ ID NO: 192 - protein, 8 aa, Artificial Sequence
  Description of Artificial Sequence Synthetic peptide
FAESMRQW

SEQ ID NO: 193 - protein, 10 aa, Artificial Sequence
  Description of Artificial Sequence Synthetic peptide
GFTFSMYRMG

SEQ ID NO: 194 - protein, 16 aa, Artificial Sequence
  Description of Artificial Sequence Synthetic peptide
SITSGGYETYADSVNG

SEQ ID NO: 195 - protein, 15 aa, Artificial Sequence
  Description of Artificial Sequence Synthetic peptide
AADSAYYGLGYDYNT

SEQ ID NO: 196 - protein, 9 aa, Artificial Sequence
  Description of Artificial Sequence Synthetic peptide
GFTFGRYMS

SEQ ID NO: 197 - protein, 16 aa, Artificial Sequence
  Description of Artificial Sequence Synthetic peptide
SITADGRTKYADSVKA

SEQ ID NO: 198 - protein, 10 aa, Artificial Sequence
  Description of Artificial Sequence Synthetic peptide
NVQTSWGNVY

SEQ ID NO: 199 - protein, 10 aa, Artificial Sequence
  Description of Artificial Sequence Synthetic peptide
GNIRSINVMG

SEQ ID NO: 200 - protein, 16 aa, Artificial Sequence
  Description of Artificial Sequence Synthetic peptide
SITSGGYETYADSVNG

SEQ ID NO: 201 - protein, 14 aa, Artificial Sequence
  Description of Artificial Sequence Synthetic peptide
AAGNYGAYGRTYDY

SEQ ID NO: 202 - protein, 10 aa, Artificial Sequence
  Description of Artificial Sequence Synthetic peptide
GDSFSAYAMG

SEQ ID NO: 203 - protein, 16 aa, Artificial Sequence
  Description of Artificial Sequence Synthetic peptide
SITRNGSATYTESIKG

SEQ ID NO: 204 - protein, 14 aa, Artificial Sequence
  Description of Artificial Sequence Synthetic peptide
HAVVNTAYDGIYDY

SEQ ID NO: 205 - protein, 10 aa, Artificial Sequence
  Description of Artificial Sequence Synthetic peptide
GRGFSNYAMG

SEQ ID NO: 206 - protein, 16 aa, Artificial Sequence
  Description of Artificial Sequence Synthetic peptide
SVDSGGSTSYGRSAKG

SEQ ID NO: 207 - protein, 8 aa, Artificial Sequence
  Description of Artificial Sequence Synthetic peptide
WGVSEYDY

SEQ ID NO: 208 - protein, 10 aa, Artificial Sequence
  Description of Artificial Sequence Synthetic peptide
GSIFRINSMG

SEQ ID NO: 209 - protein, 17 aa, Artificial Sequence
  Description of Artificial Sequence Synthetic peptide
EISAGGGTTFYADSVKG

SEQ ID NO: 210 - protein, 16 aa, Artificial Sequence
  Description of Artificial Sequence Synthetic peptide
AARQYYNGKTADEWTY

SEQ ID NO: 211 - protein, 10 aa, Artificial Sequence
  Description of Artificial Sequence Synthetic peptide
GSTFSITRMA

SEQ ID NO: 212 - protein, 17 aa, Artificial Sequence
  Description of Artificial Sequence Synthetic peptide
GIDWSGDSVSYGESVKG

SEQ ID NO: 213 - protein, 23 aa, Artificial Sequence
  Description of Artificial Sequence Synthetic peptide
NAYGILVGSQEEGEDYDYYGMDY

SEQ ID NO: 214 - protein, 10 aa, Artificial Sequence
  Description of Artificial Sequence Synthetic peptide
GRFLWIGSMG

SEQ ID NO: 215 - protein, 16 aa, Artificial Sequence
  Description of Artificial Sequence Synthetic peptide
GIEWDSRTNYAGFAQG

SEQ ID NO: 216 - protein, 10 aa, Artificial Sequence
  Description of Artificial Sequence Synthetic peptide
NTRRPWGTHF

SEQ ID NO: 217 - protein, 10 aa, Artificial Sequence
  Description of Artificial Sequence Synthetic peptide
GLTFRSYALA

SEQ ID NO: 218 - protein, 17 aa, Artificial Sequence
  Description of Artificial Sequence Synthetic peptide
TISSDGIHKYYSDSIKG

SEQ ID NO: 219 - protein, 16 aa, Artificial Sequence
  Description of Artificial Sequence Synthetic peptide
YAIPYYSDDEENLYDY

SEQ ID NO: 220 - protein, 11 aa, Artificial Sequence
  Description of Artificial Sequence Synthetic peptide
GFDFRNSWMYW

SEQ ID NO: 221 - protein, 16 aa, Artificial Sequence
  Description of Artificial Sequence Synthetic peptide
SFTSGGSTNYADSVKG

SEQ ID NO: 222 - protein, 23 aa, Artificial Sequence
  Description of Artificial Sequence Synthetic peptide
AADPRATAQAMGVRHILGYSMDY

SEQ ID NO: 223 - protein, 10 aa, Artificial Sequence
  Description of Artificial Sequence Synthetic peptide
GFAFSKYTMN

SEQ ID NO: 224 - protein, 17 aa, Artificial Sequence
  Description of Artificial Sequence Synthetic peptide
SISTDSYGTNYAAFVKG

SEQ ID NO: 225 - protein, 16 aa, Artificial Sequence
  Description of Artificial Sequence Synthetic peptide
HFLLSSHDSLGPFYDY

SEQ ID NO: 226 - protein, 10 aa, Artificial Sequence
  Description of Artificial Sequence Synthetic peptide
KTKLTFVNWA

SEQ ID NO: 227 - protein, 16 aa, Artificial Sequence
  Description of Artificial Sequence Synthetic peptide
GISSGGSTNYADSVKG

SEQ ID NO: 228 - protein, 23 aa, Artificial Sequence
  Description of Artificial Sequence Synthetic peptide
NAYGILVGSQEEGEDYDYYGMDY

SEQ ID NO: 229 - protein, 10 aa, Artificial Sequence
  Description of Artificial Sequence Synthetic peptide
GFTFAAYGMN

SEQ ID NO: 230 - protein, 16 aa, Artificial Sequence
  Description of Artificial Sequence Synthetic peptide
AITTDGSTNYRGSVKG

SEQ ID NO: 231 - protein, 16 aa, Artificial Sequence
  Description of Artificial Sequence Synthetic peptide
YAIPYYSDSEENLYDY

SEQ ID NO: 232 - protein, 10 aa, Artificial Sequence
  Description of Artificial Sequence Synthetic peptide
GNIFRNSIMH

SEQ ID NO: 233 - protein, 17 aa, Artificial Sequence
  Description of Artificial Sequence Synthetic peptide
AINKNGDSVSYGNSVKG

SEQ ID NO: 234 - protein, 19 aa, Artificial Sequence
  Description of Artificial Sequence Synthetic peptide
YATVFLSPPDDEDEGLLEL

SEQ ID NO: 235 - protein, 10 aa, Artificial Sequence
  Description of Artificial Sequence Synthetic peptide
GSTFSINNMA

SEQ ID NO: 236 - protein, 16 aa, Artificial Sequence
  Description of Artificial Sequence Synthetic peptide
SVTEGGRVNYASSVRG

SEQ ID NO: 237 - protein, 11 aa, Artificial Sequence
  Description of Artificial Sequence Synthetic peptide
HAVGRLGWGDY

SEQ ID NO: 238 - protein, 10 aa, Artificial Sequence
  Description of Artificial Sequence Synthetic peptide
GRTFRPYAMG

SEQ ID NO: 239 - protein, 16 aa, Artificial Sequence
  Description of Artificial Sequence Synthetic peptide
TITRGGYTNYADSVKG

SEQ ID NO: 240 - protein, 13 aa, Artificial Sequence
  Description of Artificial Sequence Synthetic peptide
HGRGSRWSGVNDY

SEQ ID NO: 241 - protein, 11 aa, Artificial Sequence
  Description of Artificial Sequence Synthetic peptide
GFIFDDYAMAW

SEQ ID NO: 242 - protein, 17 aa, Artificial Sequence
  Description of Artificial Sequence Synthetic peptide
GINSGGDSTTYADSVKG

SEQ ID NO: 243 - protein, 23 aa, Artificial Sequence
  Description of Artificial Sequence Synthetic peptide
NAYGILVGSQEEGEDYDYYGMDY

SEQ ID NO: 244 - protein, 10 aa, Artificial Sequence
  Description of Artificial Sequence Synthetic peptide
GDTSSFDRMG

SEQ ID NO: 245 - protein, 16 aa, Artificial Sequence
  Description of Artificial Sequence Synthetic peptide
VITVDGTTRYSDSVKG

SEQ ID NO: 246 - protein, 23 aa, Artificial Sequence
  Description of Artificial Sequence Synthetic peptide
NAYGILVGSQEEGEDYDYYGMDY

SEQ ID NO: 247 - protein, 10 aa, Artificial Sequence
  Description of Artificial Sequence Synthetic peptide
GFTFGSVNMA

SEQ ID NO: 248 - protein, 16 aa, Artificial Sequence
  Description of Artificial Sequence Synthetic peptide
GIAEDGREYYAESVKG

SEQ ID NO: 249 - protein, 18 aa, Artificial Sequence
  Description of Artificial Sequence Synthetic peptide
NLQELTLSIYGQWSHRDY

SEQ ID NO: 250 - protein, 11 aa, Artificial Sequence
  Description of Artificial Sequence Synthetic peptide
GRSITMYTMDW

SEQ ID NO: 251 - protein, 16 aa, Artificial Sequence
  Description of Artificial Sequence Synthetic peptide
TITSSGKPNYANSVKG

SEQ ID NO: 252 - protein, 16 aa, Artificial Sequence
  Description of Artificial Sequence Synthetic peptide
HALLSTHSELGPFYDY

SEQ ID NO: 253 - protein, 10 aa, Artificial Sequence
  Description of Artificial Sequence Synthetic peptide
ESAFRTYAIG

SEQ ID NO: 254 - protein, 17 aa, Artificial Sequence
  Description of Artificial Sequence Synthetic peptide
AVSAFGGGTLYADSVKG

SEQ ID NO: 255 - protein, 24 aa, Artificial Sequence
  Description of Artificial Sequence Synthetic peptide
AARFPTFYDGLWRNTAPSPTLYEY

SEQ ID NO: 256 - protein, 10 aa, Artificial Sequence
  Description of Artificial Sequence Synthetic peptide
GHAFDDLAIG

SEQ ID NO: 257 - protein, 17 aa, Artificial Sequence
  Description of Artificial Sequence Synthetic peptide
AQSGDGANTAYSAFATD

SEQ ID NO: 258 - protein, 9 aa, Artificial Sequence
  Description of Artificial Sequence Synthetic peptide
HTATDRSDY

SEQ ID NO: 259 - protein, 10 aa, Artificial Sequence
  Description of Artificial Sequence Synthetic peptide
GFTFSSYAMS

SEQ ID NO: 260 - protein, 17 aa, Artificial Sequence
  Description of Artificial Sequence Synthetic peptide
SINSEGRLTYYAHFVKG

SEQ ID NO: 261 - protein, 6 aa, Artificial Sequence
  Description of Artificial Sequence Synthetic peptide
KAGVFY

SEQ ID NO: 262 - protein, 117 aa, Artificial Sequence
  Description of Artificial Sequence Synthetic polypeptide
QVQLVESGGGLVQAGGSLRLSCVVSGSTFSMAAWAWYRQAPGKQLELVASITAGGHTNYADSVEGRFTISRDNAKNTVYLQMNALEPEDTAVYRCFADVYGSDRPSWGRGTLVTVSS

SEQ ID NO: 263 - protein, 117 aa, Artificial Sequence
  Description of Artificial Sequence Synthetic polypeptide
QVQLVESGGGLVQAGGSLRLSCAASGSTFSINAIAWYRQAPGKQLELVASITAGGHTNYADSVEGRFTISRDNAKNTVYLQMNALEPEDTAVYRCFADVYGSDRPSWGRGTLVTVSS

SEQ ID NO: 264 - protein, 119 aa, Artificial Sequence
  Description of Artificial Sequence Synthetic polypeptide
QVQLVESGGGTVQAGESLRLSCAASGRTWGMVVMGWFRQAPGKEREFVAAINWNGGSTQYGNSVKGRFTISRDNAKNTVSLQMNSLKPEDTAVYYCNAVEFGWGGYDYWGQGTLVTVSS

SEQ ID NO: 265 - protein, 117 aa, Artificial Sequence
  Description of Artificial Sequence Synthetic polypeptide
QVQLVESGGGLVQAGESLRLTCSASSVGFKINAMAWYRQAPGKQLELVASITAGGHTNYADSVEGRFTISRDDAKNTVYLQMNALEPEDTAVYRCFADVYGSDRPSWGRGTQVTVSS

SEQ ID NO: 266 - protein, 124 aa, Artificial Sequence
  Description of Artificial Sequence Synthetic polypeptide
QVQLVESGGGLVQGGGSLRLSCAASGGLFSLSAMGWYRQTPGKERELVATITLGGSTTYGDSVKGRFTISRDNSINTVYLQMNTLRPEDTAVYYCNADASRWTGDRWTAYDRVWGQGTLVTVSS

SEQ ID NO: 267 - protein, 117 aa, Artificial Sequence
  Description of Artificial Sequence Synthetic polypeptide
QVQLVESGGGMVQAGGSLRLSCVASESIDSLAAMGWFRQAPGKQLELVATISKEGHINYADSVKGRFTISRDNAKNTVYLQMNSLEPEDTAVYRCFADVYGSDRPSWGRGTLVTVSS

SEQ ID NO: 268 - protein, 124 aa, Artificial Sequence
  Description of Artificial Sequence Synthetic polypeptide
QVQLVESGGGLVQPGGSLRLSCAASGGLFSLSAMGWYRQTPGKERELVATITLGGSTTYGDSVKGRFTISRDNSINTVYLQMNTLRPEDTAVYYCNADASRWTGDRWTAYDRVWGQGTLVTVSS

SEQ ID NO: 269 - protein, 121 aa, Artificial Sequence
  Description of Artificial Sequence Synthetic polypeptide
QVQLVESGGGLVQAGDSLRLSCAASGFTFSSYLMYWVRQAPGKGLEWVSAIQPGGGSTYYADSVKGRFTISRDNAANTVYLEMNSLKPEDTAVYYCNAEWGYYSGAYRSYWGQGTQVTVSS

SEQ ID NO: 270 - protein, 117 aa, Artificial Sequence
  Description of Artificial Sequence Synthetic polypeptide
QVQLVESGGGLVQAGDSLRLSCAASGRDSSRDAMAWFRQAPGKQLELVASITAGGHTNYADSVEGRFTISRDNAKNTVYLQMNALEPEDTAVYRCFADVYGSDRPSWGRGTLVTVSS

SEQ ID NO: 271 - protein, 117 aa, Artificial Sequence
  Description of Artificial Sequence Synthetic polypeptide
QVQLVESGGGLVQPGGSLRLSCAASGFTLPYYAIVWFRQAPGKQLELVASITAGGHTNYADSVEGRFTISRDNAKNTVYLQMNALEPEDTAVYRCFADVYGSDRPSWGRGTLVTVSS

SEQ ID NO: 272 - protein, 119 aa, Artificial Sequence
  Description of Artificial Sequence Synthetic polypeptide
QVQLVESGGGLVQAGGSLRLSCSVSGRTTPNIYAMSWFRQAPGKALEWVSTINSGGFSPAYADSVKGRFTVSRDNDKNMLYLQMNMLKPEDSGLYYCARHMGYEAMDYWGKGTLVTVSS

SEQ ID NO: 273 - protein, 125 aa, Artificial Sequence
  Description of Artificial Sequence Synthetic polypeptide
QVQLVESGGDSVQPGGSLRLSCTASGRIFSINAMGWFRQAPGKERQFVAAIAMTGSTSYGDSVKGRFAISRDGAKNTVYLQMNSLKPEDTAVYYCYATVFLSPPDDEDEGLLELWGQGTTVTVSS

SEQ ID NO: 274 - protein, 117 aa, Artificial Sequence
  Description of Artificial Sequence Synthetic polypeptide
QVQLVESGGGLVQPGGSLRLSCAASGSIFSINVRAWYRQAPGKQLELVASITAGGHTNYADSVEGRFTISRDNAKNTVYLQMNALEPEDTAVYRCFADVYGSDRPSWGRGTLVTVSS

SEQ ID NO: 275 - protein, 125 aa, Artificial Sequence
  Description of Artificial Sequence Synthetic polypeptide
QVQLVESGGGLVQAGGSLRLSCSVSGNTFSINAMNWFRQAPGKQREFVATISSDGTANYAGSVKGRFTISKDPGVSRVHLRMDGLKPEDTAVYYCYVVAAVFKEDYDSPPFHDYWGQGTLVTVSS

SEQ ID NO: 276 - protein, 117 aa, Artificial Sequence
  Description of Artificial Sequence Synthetic polypeptide
QVQLMESGGGLVQAGGSLRLSCSASSSISSINVMAWYRQAPGKQLELVATISKEGHINYADSVKGRFTISRDNAKNTVYLQMNSLEPEDTAVYRCFADVYGSDRPSWGRGTLVTVSS

SEQ ID NO: 277 - protein, 123 aa, Artificial Sequence
  Description of Artificial Sequence Synthetic polypeptide
QVQLVESGGGLVEPGGSLRLSCAASGRTLSRYTMAWFRQAPGKEREFVAAINWSGDRVSYTDSVKGRFTVSRDNAKNTVYLQMNSLKPEDTAVYYCYAIPYYSDSEENLYDYWGQGTLVTVSS

SEQ ID NO: 278 - protein, 118 aa, Artificial Sequence
  Description of Artificial Sequence Synthetic polypeptide
QVQLVESGGGLVQPGGSLRLSCAAASESRFHYNTMAWFRQAPGKQLELVASITAGGHTNYADSVEGRFTISRDNAKNTVYLQMNALEPEDTAVYRCFADVYGSDRPSWGRGTLVTVSS

SEQ ID NO: 279 - protein, 120 aa, Artificial Sequence
  Description of Artificial Sequence Synthetic polypeptide
QVQLVESGGGLVQAGGSLRLSCAVSGSGRSINVMGWYRQPPGRQREQRELVALIRSGGSTDYADSVKDRFTISRDNAKNILYLQMNSLTPEDTAVYYCNAERYSRALRPWGQGTLVTVSS

SEQ ID NO: 280 - protein, 119 aa, Artificial Sequence
  Description of Artificial Sequence Synthetic polypeptide
QVQLVESGGGLVQAGGSLRLSCAASGRTFSFSMYNMAWFRQAPGKEREFVAAISWSGSSADYADFVKGRFTISRDIAANTVSLQMNSLKPEDTAVYNCYAVRIDGMDYWGKGTLVTVSS

SEQ ID NO: 281 - protein, 125 aa, Artificial Sequence
  Description of Artificial Sequence Synthetic polypeptide
QVQLVESGGGLVQAGGSLRLSCAASEATFSKYVMGWFRQAPGKEREFVATIDWKGSSTHYADSVEGRFTISKDNAKSTVWLQMNSLKPEDTAVYYCAADLALSMRYYHAPNYEYWGQGTLVTVSS

SEQ ID NO: 282 - protein, 126 aa, Artificial Sequence
  Description of Artificial Sequence Synthetic polypeptide
QVQLVESGGALVQPGGSLRLSCAASGFTFSTYWMHWVRQAPGKGLEWVSAINSDSSALYADSVKGRFAISRDNAKNTVYLQMNSLKPEDTAKYYCSASIGVRPYSGSDLLRRLEVWGQGTLVTVSS

SEQ ID NO: 283 - protein, 125 aa, Artificial Sequence
  Description of Artificial Sequence Synthetic polypeptide
QVQLVESGGGLVQPGGSLRLSCAASEATFSRYVMGWFRQAPGKEREFVAAIDWKGSSTHYADSVEGRFTISRDNAKSTVWLQMNSLKPEDTAVYYCAADLALSMRYYHAPNYEYWGQGTQVTVSS

SEQ ID NO: 284 - protein, 117 aa, Artificial Sequence
  Description of Artificial Sequence Synthetic polypeptide
QVQLVESGGGLVQAGGSLRLSCTPSGRSFSPYTMGWFRQAPGKEREFVAASSASGGSTTYASSGKDRFTISRDRAKNTVYLQMNSLKPEDTAVYYCYVNIWNGGDYWGQGTLVTVSS

SEQ ID NO: 285 - protein, 122 aa, Artificial Sequence
  Description of Artificial Sequence Synthetic polypeptide
QVQLVESGGGLVQPGGSLRLSCAASTSIFNMNAMNWYRQAPGKGLEWISTINSGGTYTSYADSVKGRFTISSDNAKNTVYLQMNGLGPEDTAVYYCNAVVNTILDFLPKNYWGKGTLVTVSS

SEQ ID NO: 286 - protein, 124 aa, Artificial Sequence
  Description of Artificial Sequence Synthetic polypeptide
QVQLVESGGGSVRPGGSLKLSCVLSGGSSDQYTIGWIRQAPGKEREALLASSRSGGSLAYNDSIKDRFIISRDNTKNTVYLRLRRLEPEDTGVYYCAAGFGPAVPVTPDEYTYWGQGTLVTVSS

SEQ ID NO: 287 - protein, 116 aa, Artificial Sequence
  Description of Artificial Sequence Synthetic polypeptide
QVQLVESGGGLVQAGGSLRLSCAYSGNTSSIVDMEWYRQAPGKQRDLVAFIRRAGTTVYASSVKGRFTISTDKIKKTMSLQMNSLQPEDTAVYICNANDWGGREYWGQGTQVTVSS

SEQ ID NO: 288 - protein, 111 aa, Artificial Sequence
  Description of Artificial Sequence Synthetic polypeptide
QVQLVESGGGLVQAGGSLRLSCAASGSIFSYSTMGWYRQAPGKQRELVASIPRAGTRTYLDSVKGRFAISRDTAKNTVYLQMNSLKPEDTAVYYCRGDNYWGQGTLVTVSS

SEQ ID NO: 289 - protein, 120 aa, Artificial Sequence
  Description of Artificial Sequence Synthetic polypeptide
QVQLVESGGGLVQPGGSLRLSCAASGFTFSRYWMYWVRQAPGKGLEWVSTINTGGGSTYYADSVKGRFTISRDNAKNTLYLLMNRLKSEDTAVYYCAKGHSNYEDALDAWGQGTLVTVSS

SEQ ID NO: 290 - protein, 122 aa, Artificial Sequence
  Description of Artificial Sequence Synthetic polypeptide
QVQLVESGGGLVQPGGSLRLSCAASGFTFDNYFLSWVRQAPGKGLEWVAGTYGAIDATYYEDSVKGRFTISSDSAKNTVYLQMDSLKPADTAVYYCAAGRLVTSTTSSYDYWGRGTLVTVSS

SEQ ID NO: 291 - protein, 124 aa, Artificial Sequence
  Description of Artificial Sequence Synthetic polypeptide
QVQLVESGGGLVQAGGSMKLTCTASGRTVDDDNMGWFRRGPGKEREVVATINSRGWEIYYSDSVKGRFTISRDNAKNSVYLQMTSLKSEDTAVYYCAAGPYGTGWNLDNQYTYWGQGTQVTVSS

SEQ ID NO: 292 - protein, 124 aa, Artificial Sequence
  Description of Artificial Sequence Synthetic polypeptide
QVQLVESGGGLVQPGGSLRLSCVVSGGKSDQYTIAWIRQAPGKEREALLASSRSGGSLTYIDSIKDRFIISRDNAKNTVYLRMRSLKPEDTGVYYCAAGYGPAVPVTPDEYTYWGQGTLVTVSS

SEQ ID NO: 293 - protein, 117 aa, Artificial Sequence
  Description of Artificial Sequence Synthetic polypeptide
QVQLVESGGGWVQAGGSLRLSCAASGSIFGINVMAWYRQAPGKQLDLVATISAGGIINYADFVEGRFTISRDNSKNTVYLQMNSLEPEDTAVYRCFADVAGSDRPSWGQGTLVTVSS

SEQ ID NO: 294 - protein, 123 aa, Artificial Sequence
  Description of Artificial Sequence Synthetic polypeptide
QVQLVESGGGSVQAGGSLRLSCSASGRTLSNYNVGWFRTGEGKDREFVAAISQSGDMTSYGDSVKGRFTISRDNAKNTVYLQMKSLEPEDTAVYYCAKLDAEGYGGDRYLEVWGQGTLVTVSS

SEQ ID NO: 295 - protein, 122 aa, Artificial Sequence
  Description of Artificial Sequence Synthetic polypeptide
QVQLVESGGGLVQAGGSLTLSCAASGLTFDDVPIGWFRQAPGKEREGVSRIEVSDDSTYYADSVKGRFTISSDSVKNMVYLQMNDLKPEDTAVYYCAADSSYYGLGYDYNTWGQGTLVTVSS

SEQ ID NO: 296 - protein, 114 aa, Artificial Sequence
  Description of Artificial Sequence Synthetic polypeptide
QVQLVESGGGLVQAGDSLRLSCAASRSIFSIAMMGWYRQAPGKEREFVATITTDGNTNYADFVKGRFTISRVNARNTVYLQMNSLKPEDTALYICIARKYGTYWGQGTLVTVSS

SEQ ID NO: 297 - protein, 116 aa, Artificial Sequence
  Description of Artificial Sequence Synthetic polypeptide
QVQLVESGGGLVQAGDSLRLSCAATTRPSNLIGMGWFRQAPGTEREFVAAIRWSGGTAYYADSVKGRFTISRDNAKNTVYLQMNSLKPEDTAVYYCKAVDGGLGYWGQGTQVTVSS

SEQ ID NO: 298 - protein, 125 aa, Artificial Sequence
  Description of Artificial Sequence Synthetic polypeptide
QVQLVESGGGLVQAGDSLRLSCAASGSIASALNAMGWGRQAPGKQRELVASITSGGTTTYAKSVKGRFTISRDNAKNMVYLEMNNLKPEDTAVYHCAASHFDYRDYAFTSEYDYWGQGTLVTVSS

SEQ ID NO: 299 - protein, 117 aa, Artificial Sequence
  Description of Artificial Sequence Synthetic polypeptide
QVQLVESGGGLVQAGDSLRLSCAAAGDTLDKYHIGWFRQAPGKERRFVAAIKWSDGDTSYARSVKGRFTISTDNAKNTAYLQMNSLKSEDTAVYYCYVVTVRTGDYWGQGTLVTVSS

SEQ ID NO: 300 - protein, 117 aa, Artificial Sequence
  Description of Artificial Sequence Synthetic polypeptide
QVQLVESGGGLVQPGGSLSVSCVVSGTTSRINTMAWFREAAGKEREFVAAIVINGGTTSYGASVKGRFTISRHNAENMVYLQMNGLQPEDTAVYYCHAISVPDGDYWGQGTLVTVSS

SEQ ID NO: 301 - protein, 111 aa, Artificial Sequence
  Description of Artificial Sequence Synthetic polypeptide
QVQLVESGGGLVQAGGSLRLSCAASGSIFSYSTMGWYRQAPGKQRELVASIPRAGTRTYLDSVKGRFAISRDTAKNTVYLQMNSLKPEDTAVYYCRGDNYWGQGTQVTVSS

SEQ ID NO: 302 - protein, 122 aa, Artificial Sequence
  Description of Artificial Sequence Synthetic polypeptide
QVQLVESGGGLVQAGDSLRLSCAASGLTFSMYTMAWFRQAPGKGLEWVSTINFDVVTTTYADSVKGRFTISRGNTKNKVYLEMNSLKPEDTAVYYCAVGDFYSRSQRQYTYWGQGTLVTVSS

SEQ ID NO: 303 - protein, 117 aa, Artificial Sequence
  Description of Artificial Sequence Synthetic polypeptide
QVQLVESGGGLVQPGGSLRLSCAASGPIVRFTAMGWYRQAPGKQLELVATISKEGHINYADSVKGRFTISRDNAKNTVYLQMNSLEPEDTAVYRCFADVYGSDRPSWGRGTLVTVSS

SEQ ID NO: 304 - protein, 117 aa, Artificial Sequence
  Description of Artificial Sequence Synthetic polypeptide
QVQLVESGGRLVQAGDSLRLSCAASGPTFNPYAMAWFRQAPGKQLELVASITAGGHTNYADSVEGRFTISRDNAKNTVYLQMNALEPEDTAVYRCFADVYGSDRPSWGRGTQVTVSS

SEQ ID NO: 305 - protein, 123 aa, Artificial Sequence
  Description of Artificial Sequence Synthetic polypeptide
QVQLVESVGGLVQPGGSLRLSCAASGFTFSHYQMYWVRQAPGKGLQWISSIHTDGRNTYYQNSVKGRFTISRDNAKNTVYLQMNSLKPEDTAVYYCYAIPYYSDSEENLYDYWGQGTLVTVSS

SEQ ID NO: 306 - protein, 118 aa, Artificial Sequence
  Description of Artificial Sequence Synthetic polypeptide
QVQLVESGGGLVQPGGSLRLSCAASGSIFSINAMGWYRQAPGQQREMVAVITRPGTTNYAESVKGRFTISRDKNTVYLQMNGLKPEDTAVYYCNAQSGNYMLGFYDYWGQGTLVTVSS

SEQ ID NO: 307 - protein, 123 aa, Artificial Sequence
  Description of Artificial Sequence Synthetic polypeptide
QVQLVESGGGLVQTGGSLRLACATSGRAVNRHIMGWFRQAPGKEREFVASISWNSGRTYFADFVKGRFTISRDNAKVYLQMNSLKPEDTAVYYCRADYYVDYEDDRMGFFDAWGQGTQVTVSS

SEQ ID NO: 308 - protein, 135 aa, Artificial Sequence
  Description of Artificial Sequence Synthetic polypeptide
QVQLVESGGGLVQSGGSLTLSCAASGFTSDYYAIGWFRQAQGKEREFVATVPWNDGIGGSKHYAEVVKGRFTISRDMYKNMVYLQMNSLLPDDTAVYYCAAAGPYRLYPEGHYPPDVNAYEYAYWGQGTLVTVSS

SEQ ID NO: 309 - protein, 125 aa, Artificial Sequence
  Description of Artificial Sequence Synthetic polypeptide
QVQLVESGGGQMQAGGSLKLSCEASGRSVVFSTMGMAWFRQAPGKAREFVAGVGVKGGSTTYRESVKGRFSISRDNDKNSVDLQMNSLKPEDTAVYYCYAIPYYSDSEENLYDYWGQGTLVTVSS

SEQ ID NO: 310 - protein, 118 aa, Artificial Sequence
  Description of Artificial Sequence Synthetic polypeptide
QVQLVESGGGLVQAGGSLRLSCAASGSTFNIETMSWFRQAPGNQREMVSSISVGGFPPYYTHSVKGRFTISRDEAKNILYLQMNSLKPEDTAVYFCNAYSRAGSKYYWGQGTLVTVSS

SEQ ID NO: 311 - protein, 124 aa, Artificial Sequence
  Description of Artificial Sequence Synthetic polypeptide
QVQLVESGGGTVQPGDSLRLSCAASGFTLADYGIGWFRQAPGKGREGVACMNKNDGNTFYAESVKGRFQISRDNAENTMYLQMNNLNSEDTAVYYCAADILGYDCSGSASMDHWGKGTLVTVSS

SEQ ID NO: 312 - protein, 122 aa, Artificial Sequence
  Description of Artificial Sequence Synthetic polypeptide
QVQLVESGGGLVQPGKSLRLSCAASGFDFSSSRMYWVRQAPGEGLEWIASINAGGNTRDYSGSVKGRFTISRDNAKNTVYLQMNDLKPEDTAKYYCYGELKSASRFRVDNYWGQGTLVTVSS

SEQ ID NO: 313 - protein, 123 aa, Artificial Sequence
  Description of Artificial Sequence Synthetic polypeptide
QVQLVESGGGSVQPGGSLRLSCTISGFAAEGYAITWFRQAPGEERELVAHIRDGGITNYTDSVKGRFTISRDNAKNEVYLQMDGLKPEDTAVYYCNAGGITFSSGTFYPDPYWGQGTLVTVSS

SEQ ID NO: 314 - protein, 124 aa, Artificial Sequence
  Description of Artificial Sequence Synthetic polypeptide
QVQLVESVGGLVQAGGSLRLSCQASGFTFSNHAMSWVRQAPGKGLEWVSFVSNGGRNTYYADSVKGRFTISRDGAKNTVYLQMDSLKPEDTAVYYCAASTTVFTGTTPGAYNYWGQGTQVTVSS

SEQ ID NO: 315 - protein, 124 aa, Artificial Sequence
  Description of Artificial Sequence Synthetic polypeptide
QVQLVESGGGLVQAGESLRLSCAVSGRSSSRYTMAWFRQAPGKERNLIAALSQNGVTTYYADSVKGRFSISRDHTKNMVYLQMNTLKPEDTALYYCGAAPSGALVQDINHYYYWGQGTLVTVSS

SEQ ID NO: 316 - protein, 117 aa, Artificial Sequence
  Description of Artificial Sequence Synthetic polypeptide
QVQLVESGGGSVQAGGSLSLSCTASGFIFSIRTMAWYRQAPGKQLELVASITAGGHTNYADSVEGRFTISRDNAKNTVYLQMNALEPEDTAVYRCFADVYGSDRPSWGRGTLVTVSS

SEQ ID NO: 317 - protein, 123 aa, Artificial Sequence
  Description of Artificial Sequence Synthetic polypeptide
QVQLVESGGGLVQPGGSLRLSCAASGSFYSINDMGWYRQAPGKQRELVATITGRGRTNYKDSVKGRFAISRDNAKNTVYLQMNSLKPEDTAVYYCNVRAVVRILTHYRGSEVWGQGTLVTVSS

SEQ ID NO: 318 - protein, 123 aa, Artificial Sequence
  Description of Artificial Sequence Synthetic polypeptide
QVQLVESGGGSVQAGASLRLSCAASGRTLSDYTMAWFRQPPGKEREFVAAIGWNGGNIYYADSVKGRFTVSRDNAKNTVYLQMNSLKPEDTAVYYCYAIPYYSDSEENLYDYWGQGTLVTVSS

SEQ ID NO: 319 - protein, 117 aa, Artificial Sequence
  Description of Artificial Sequence Synthetic polypeptide
QVQLVESGGGLVQAGGSLTLSCAASGRPGITYTMGWFRQAPGKEREFVAGISGSGGSTAYADSVKGRFTISRDNAKNTVYLQMNSLKPEDTAVYYCLIVPRDGMDYWGKGTLVTVSS

SEQ ID NO: 320 - protein, 124 aa, Artificial Sequence
  Description of Artificial Sequence Synthetic polypeptide
QVQLVESGGGLVQPGGSLRLSCAASGFTLADYGIGWFRQAPGKGREGVACMNKNDGNTFYAESVKGRFQISRDNAENTMYLQMNNLNSEDTAVYYCAADILGYDCSGSASMDHWGKGTLVTVSS

SEQ ID NO: 321 - protein, 123 aa, Artificial Sequence
  Description of Artificial Sequence Synthetic polypeptide
QVQLVESGGGLVQAGESLRLSCLASGFAFSDYAMNWVRQAPGKGLEWVSAISWNGGSTYYAESIKGRFTISRDNAKNTLYLQMNSLKSEDTAVYYCAKSSTWHGRYYYGMDYWGKGTLVTVSS

SEQ ID NO: 322 - protein, 122 aa, Artificial Sequence
  Description of Artificial Sequence Synthetic polypeptide
QVQLVESGGGTVKPGGSLRLSCAASGLTFSMYSMGWFRQAPGKGLEWVSSINPGGGSTVYADSVKGRFSISRDNAKNMVYLQMNSLEPEDTAVYYCGATIVELGRGYSTDYWGEGTLVTVSS

SEQ ID NO: 323 - protein, 118 aa, Artificial Sequence
  Description of Artificial Sequence Synthetic polypeptide
QVQLVESGGGLVRTGDSLRLSCTASGRSFGTYAVTWFRQVPGKEREFVAAISNSATTIQYTHSVKGRFTISRDNAKNTVYLQMNSLKPEDTAVYYCHAVAWDYGLDHWGQGTLVTVSS

SEQ ID NO: 324 - protein, 117 aa, Artificial Sequence
  Description of Artificial Sequence Synthetic polypeptide
QVQLVESGGGLVQPGGSLRLSCAASGFTSDYYVIGWFRQAPGKQLELVATISKEGHINYADSVKGRFTISRDNAKNTVYLQMNSLEPEDTAVYRCFADVYGSDRPSWGRGTLVTVSS

SEQ ID NO: 325 - protein, 115 aa, Artificial Sequence
  Description of Artificial Sequence Synthetic polypeptide
EVQLVESGGGLVQPGGSLRLSCAASGSIFGINVMAWYRQAPGKGLEWISTIYNDASNTDYADSVKGRFTVSRDNSKNTLYLQMNSLRAEDTAKYYCFAESMRQWWGQGNQVTVSS

SEQ ID NO: 326 - protein, 121 aa, Artificial Sequence
  Description of Artificial Sequence Synthetic polypeptide
EVQLVESGGGLVQPGGSLRLSCAASGFTFSMYRMGWFRQAPGKGLEWVASITSGGYETYADSVNGRFVISRDNSKNTLYLQMNSLRAEDTAVYYCAADSAYYGLGYDYNTWGQGTLVTVSS

SEQ ID NO: 327 - protein, 115 aa, Artificial Sequence
  Description of Artificial Sequence Synthetic polypeptide
EVQLVESGGGLVQAGDSLRLSCEASGFTFGRYMSWVRQAPGKGLEWVASITADGRTKYADSVKARFHISRDNSKNTLYLQMNSLRAEDTAVYYCNVQTSWGNVYWGQGTLVTVSS

SEQ ID NO: 328 - protein, 120 aa, Artificial Sequence
  Description of Artificial Sequence Synthetic polypeptide
EVQLVESGGGLVQPGGSLRLSCAASGNIRSINVMGWYRQAPGKGLEWVASITSGGYETYADSVNGRFVISRDNSKNTLYLQMNSLRAEDTAVYYCAAGNYGAYGRTYDYWGQGTLVTVSS

SEQ ID NO: 329 - protein, 120 aa, Artificial Sequence
  Description of Artificial Sequence Synthetic polypeptide
EVQLVESGGGLVQPGGSLRLSCAVSGDSFSAYAMGWFRQAPGKGLEWVASITRNGSATYTESIKGRFTISRDNSKNTLYLQMNSLRAEDTAVYYCHAVVNTAYDGIYDYWGQGTLVTVSS

SEQ ID NO: 330 - protein, 114 aa, Artificial Sequence
  Description of Artificial Sequence Synthetic polypeptide
EVQLVESGGGLVQPGGSLRLSCAASGRGFSNYAMGWFRQAPGKGREWVASVDSGGSTSYGRSAKGRFTVSRDNSKNTLYLQMNSLRAEDTAVYYCWGVSEYDYWGQGTLVTVSS

SEQ ID NO: 331 - protein, 123 aa, Artificial Sequence
  Description of Artificial Sequence Synthetic polypeptide
EVQLVESGGGLVQPGRSLRLTCVVTGSIFRINSMGWYRQAPGKGLEWVAEISAGGGTTFYADSVKGRATISRDNSKNTLYLQMNSLRAEDTAVYYCAARQYYNGKTADEWTYWGQGTQVTVSS

SEQ ID NO: 332 - protein, 130 aa, Artificial Sequence
  Description of Artificial Sequence Synthetic polypeptide
EVQLVESGGGLVQPGGSLRLSCTASGSTFSITRMAWYRQAPGKGLEWVAGIDWSGDSVSYGESVKGRFTISRDNSKNTLYLQMNSLRAEDTAVYYCNAYGILVGSQEEGEDYDYYGMDYWGNGTLVTVSS

SEQ ID NO: 333 - protein, 116 aa, Artificial Sequence
  Description of Artificial Sequence Synthetic polypeptide
EVQLVESGGGLVQAGGSLRLSCLVSGRFLWIGSMGWYRQAPGKGREWVAGIEWDSRTNYAGFAQGRFTISRDNSKNTLYLQMNSLRAEDTAVYYCNTRRPWGTHFWGQGTQVTVSS

SEQ ID NO: 334 - protein, 123 aa, Artificial Sequence
  Description of Artificial Sequence Synthetic polypeptide
EVQLVESGGGLVQPGDSLRLSCAASGLTFRSYALAWFRQAPGKGREWVSTISSDGIHKYYSDSIKGRFFVSRDNSKNTLYLQMNSLRAEDTAVYYCYAIPYYSDDEENLYDYWGQGTQVTVSS

SEQ ID NO: 335 - protein, 129 aa, Artificial Sequence
  Description of Artificial Sequence Synthetic polypeptide
EVQLVESGGGLVQPGGSLRLSCVASGFDFRNSWMYWWRQAPGKGLEWVASFTSGGSTNYADSVKGRFSISRDNSKNTLYLQMNSLRAEDTAVYYCAADPRATAQAMGVRHILGYSMDYWGKGTLVTVSS

SEQ ID NO: 336 - protein, 123 aa, Artificial Sequence
  Description of Artificial Sequence Synthetic polypeptide
EVQLVESGGGLVQPGGSLRLSCAASGFAFSKYTMNWFRQAPGKGLEWVSSISTDSYGTNYAAFVKGRFTISRDNSKNTLYLQMNSLRAEDTAVYFCHFLLSSHDSLGPFYDYWGQGTLVTVSS

SEQ ID NO: 337 - protein, 129 aa, Artificial Sequence
  Description of Artificial Sequence Synthetic polypeptide
EVQLVESGGGLVQPGGSLRLSCAASKTKLTFVNWAWYRQAPGKGREWVAGISSGGSTNYADSVKGRFTISRDNSKNTLYLQMNSLRAEDTAVYYCNAYGILVGSQEEGEDYDYYGMDYWGNGTQVTVSS

SEQ ID NO: 338 - protein, 122 aa, Artificial Sequence
  Description of Artificial Sequence Synthetic polypeptide
EVQLVESGGGLVQPGESLRLSCSASGFTFAAYGMNWVRQAPGKGREWVAAITTDGSTNYRGSVKGRFTISRDNSKNTLYLQMNSLRAEDTAVYYCYAIPYYSDSEENLYDYWGQGTLVTVSS

SEQ ID NO: 339 - protein, 126 aa, Artificial Sequence
  Description of Artificial Sequence Synthetic polypeptide
EVQLVESGGGLVQAGGSLTLSCAASGNIFRNSIMHWYRQAPGKGREWVAAINKNGDSVSYGNSVKGRFTILRDNSKNTLYLQMNSLRAEDTAVYYCYATVFLSPPDDEDEGLLELWGQGTLVTVSS

SEQ ID NO: 340 - protein, 117 aa, Artificial Sequence
  Description of Artificial Sequence Synthetic polypeptide
EVQLVESGGGLVQAGGSLRLSCVVSGSTFSINNMAWFRQAPGKGREWVASVTEGGRVNYASSVRGRFAISRDNSKNTLYLQMNSLRAEDTAVYYCHAVGRLGWGDYWGQGTLVTVSS

SEQ ID NO: 341 - protein, 119 aa, Artificial Sequence
  Description of Artificial Sequence Synthetic polypeptide
EVQLVESGGGLVQPGGSLRLSCAASGRTFRPYAMGWFRQAPGKGLEWVATITRGGYTNYADSVKGRFTISRDNSKNTLYLQMNSLRAEDTAVYYCHGRGSRWSGVNDYWGQGTLVTVSS

SEQ ID NO: 342 - protein, 130 aa, Artificial Sequence
  Description of Artificial Sequence Synthetic polypeptide
EVQLVESGGGLVQRGDSLTLSCATSGFIFDDYAMAWWRQAPGKGREWVSGINSGGDSTTYADSVKGRFTISRDNSKNTLYLQMNSLRAEDTAVYYCNAYGILVGSQEEGEDYDYYGMDYWGNGTLVTVSS

SEQ ID NO: 343 - protein, 129 aa, Artificial Sequence
  Description of Artificial Sequence Synthetic polypeptide
EVQLVESGGGLVQPGGSLTLSCAASGDTSSFDRMGWYRQAPGKGREWVAVITVDGTTRYSDSVKGRFAISRDNSKNTLYLQMNSLRAEDTAVYYCNAYGILVGSQEEGEDYDYYGMDYWGNGTQVTVSS

SEQ ID NO: 344 - protein, 124 aa, Artificial Sequence
  Description of Artificial Sequence Synthetic polypeptide
EVQLVESGGGLVQPGGSLRLSCAASGFTFGSVNMAWVRQAPGKGLEWVAGIAEDGREYYAESVKGRFTISRDNSKNTLYLQMNSLRAEDTAVYYCNLQELTLSIYGQWSHRDYWGQGTQVTVSS

SEQ ID NO: 345 - protein, 122 aa, Artificial Sequence
  Description of Artificial Sequence Synthetic polypeptide
EVQLVESGGGLVQAGDSVRLSCTVSGRSITMYTMDWWRQAPGKGREFVATITSSGKPNYANSVKGRATVDRDNSKNTLYLQMNSLRAEDTAVYSCHALLSTHSELGPFYDYWGQGTLVTVSS

SEQ ID NO: 346 - protein, 131 aa, Artificial Sequence
  Description of Artificial Sequence Synthetic polypeptide
EVQLVESGGGLVQAGGSLRLSCTASESAFRTYAIGWFRQAPGKGLEWVAAVSAFGGGTLYADSVKGRFTISRDNSKNTLYLQMNSLRAEDTAVYFCAARFPTFYDGLWRNTAPSPTLYEYWGQGTQVTVSS

SEQ ID NO: 347 - protein, 116 aa, Artificial Sequence
  Description of Artificial Sequence Synthetic polypeptide
EVQLVESGGGLVQPGESLTLSCATSGHAFDDLAIGWFRQAPGKGLEWVAAQSGDGANTAYSAFATDRFTISRDNSKNTLYLQMNSLRAEDTAVYYCHTATDRSDYWGQGTLVTVSS

SEQ ID NO: 348 - protein, 113 aa, Artificial Sequence
  Description of Artificial Sequence Synthetic polypeptide
EVQLVESGGGLVQPGGSLRLSCAASGFTFSSYAMSWVRQAPGKGLEWVASINSEGRLTYYAHFVKGRFNVSRDNSKNTLYLQMNSLRAEDTAVYYCKAGVFYWGKGTQVTVSS

SEQ ID NO: 349 - protein, 194 aa, Severe acute respiratory syndrome coronavirus 2
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG

(19) United States
(12) Patent Application Publication
(10) Pub. No.: US 2023/0220054 A1
(43) Pub. Date: Jul. 13, 2023

(54) ANTI-CORONAVIRUS ANTIBODIES AND USES THEREOF

(71) Applicant: Ab Studio Inc., Hayward, CA (US)

(72) Inventors: Jianbo Dong, Santa Clara, CA (US); Chao Bai Huang, San Leandro, CA (US); Yue Liu, Foster City, CA (US)

(21) Appl. No.: 17/996,743

(22) PCT Filed: Apr. 20, 2021
(86) PCT No.: PCT/US2021/028135

§ 371 Date: Oct. 20, 2022

Related U.S. Application Data
(60) Provisional application No. 63/069,610, filed on Aug. 24, 2020.
(60) Provisional application No. 63/012,751, filed on Apr. 20, 2020.

(51) Int. Cl. C07K 16/10; C12N 15/63; A61P 31/14

(52) U.S. Cl. CPC C07K 16/10; C12N 15/63; A61P 31/14; C07K 2317/565; C07K 2317/31

(57) ABSTRACT

This disclosure relates to anti-coronavirus (e.g., SARS-CoV-2) antibodies or antigen-binding fragments and uses thereof.

### CLAIM OF PRIORITY

This application claims the benefit of U.S. Provisional Application No. 63/012,751, filed on Apr. 20, 2020 and U.S. Provisional Application No. 63/069,610, filed on Aug. 24, 2020. The entire contents of the foregoing applications are incorporated herein by reference.

### TECHNICAL FIELD

This disclosure relates to anti-coronavirus antibodies or antigen-binding fragments and uses thereof.

### BACKGROUND

Coronaviruses are enveloped, positive-sense single-strand RNA viruses with mammalian and avian hosts. Previous coronaviruses known to infect humans include 229E, NL63, OC43, HKU1, SARS-CoV, and MERS-CoV, which cause a range of mild seasonal illnesses to severe diseases outbreaks. Notably, the past outbreaks of severe acute respiratory syndrome (SARS) (2003) and Middle East respiratory syndrome (MERS) (2012) were caused by the coronaviruses SARS-CoV and MERS-CoV, respectively (See Human Coronavirus Types: Centers for Disease Control and Prevention). SARS-CoV-2, which emerged in December 2019, is the seventh known coronavirus to infect humans, and the third coronavirus to cross species barriers and cause severe respiratory infections in humans in less than two decades after SARS and MERS. It causes the coronavirus disease 2019 (COVID-19) (See Okba N M A, Muller M A, Li W, et al. Severe Acute Respiratory Syndrome Coronavirus 2-Specific Antibody Responses in Coronavirus Disease 2019 Patients. Emerg Infect Dis. 2020 Apr. 8; 26(7)) that is more contagious than SARS-CoV and MERS-CoV.

The high rate of infection and worldwide impact caused by the disease led the World Health Organization to declare COVID-19 a pandemic. As of 13th of April 2020, the virus had confirmed infections in more than 1.8 million people worldwide, with nearly 120,000 confirmed deaths and an estimated 6.34% mortality rate (See Coronavirus disease (COVID-19) Pandemic World Health Organization: World Health Organization). Identification of the etiology of the virus, publication of studies, and international collaborative efforts have led to rapid development of real-time PCR diagnostic assays that support case ascertainment and tracking of COVID-19 outbreak. However, validated serologic diagnostic assays and therapeutics against SARS-CoV-2 are still lacking, and have become an urgent necessity to combat COVID-19.

### SUMMARY

This disclosure relates to anti-coronavirus S protein antibodies, antigen-binding fragment thereof, and the uses thereof.

In one aspect, provided herein is an antibody or antigen-binding fragment thereof that binds to coronavirus S protein, comprising a heavy chain single variable domain (VHH) comprising complementarity determining regions (CDRs) 1, 2, and 3. In some embodiments, the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence. In some embodiments, the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively;
(4) the selected VHH CDRs 1, 2, 3 amino acid sequences are set forth in SEQ ID NOs: 10, 11, and 12, respectively;
(5) the selected VHH CDRs 1, 2, 3 amino acid sequences are set forth in SEQ ID NOs: 13, 14, and 15, respectively;
(6) the selected VHH CDRs 1, 2, 3 amino acid sequences are set forth in SEQ ID NOs: 16, 17, and 18, respectively;
(7) the selected VHH CDRs 1, 2, 3 amino acid sequences are set forth in SEQ ID NOs: 19, 20, and 21, respectively;
(8) the selected VHH CDRs 1, 2, 3 amino acid sequences are set forth in SEQ ID NOs: 22, 23, and 24, respectively;
(9) the selected VHH CDRs 1, 2, 3 amino acid sequences are set forth in SEQ ID NOs: 25, 26, and 27, respectively;
(10) the selected VHH CDRs 1, 2, 3 amino acid sequences are set forth in SEQ ID NOs: 28, 29, and 30, respectively;
(11) the selected VHH CDRs 1, 2, 3 amino acid sequences are set forth in SEQ ID NOs: 31, 32, and 33, respectively;
(12) the selected VHH CDRs 1, 2, 3 amino acid sequences are set forth in SEQ ID NOs: 34, 35, and 36, respectively;
(13) the selected VHH CDRs 1, 2, 3 amino acid sequences are set forth in SEQ ID NOs: 37, 38, and 39, respectively;
(14) the selected VHH CDRs 1, 2, 3 amino acid sequences are set forth in SEQ ID NOs: 40, 41, and 42, respectively;
(15) the selected VHH CDRs 1, 2, 3 amino acid sequences are set forth in SEQ ID NOs: 43, 44, and 45, respectively;
(16) the selected VHH CDRs 1, 2, 3 amino acid sequences are set forth in SEQ ID NOs: 46, 47, and 48, respectively;
(17) the selected VHH CDRs 1, 2, 3 amino acid sequences are set forth in SEQ ID NOs: 49, 50, and 51, respectively;
(18) the selected VHH CDRs 1, 2, 3 amino acid sequences are set forth in SEQ ID NOs: 52, 53, and 54, respectively;
(19) the selected VHH CDRs 1, 2, 3 amino acid sequences are set forth in SEQ ID NOs: 55, 56, and 57, respectively;
(20) the selected VHH CDRs 1, 2, 3 amino acid sequences are set forth in SEQ ID NOs: 58, 59, and 60, respectively;
(21) the selected VHH CDRs 1, 2, 3 amino acid sequences are set forth in SEQ ID NOs: 61, 62, and 63, respectively;
(22) the selected VHH CDRs 1, 2, 3 amino acid sequences are set forth in SEQ ID NOs: 64, 65, and 66, respectively;
(23) the selected VHH CDRs 1, 2, 3 amino acid sequences are set forth in SEQ ID NOs: 67, 68, and 69, respectively;
(24) the selected VHH CDRs 1, 2, 3 amino acid sequences are set forth in SEQ ID NOs: 70, 71, and 72, respectively;
(25) the selected VHH CDRs 1, 2, 3 amino acid sequences are set forth in SEQ ID NOs: 73, 74, and 75, respectively;
(26) the selected VHH CDRs 1, 2, 3 amino acid sequences are set forth in SEQ ID NOs: 76, 77, and 78, respectively;
(27) the selected VHH CDRs 1, 2, 3 amino acid sequences are set forth in SEQ ID NOs: 79, 80, and 81, respectively;
(28) the selected VHH CDRs 1, 2, 3 amino acid sequences are set forth in SEQ ID NOs: 82, 83, and 84, respectively;
(29) the selected VHH CDRs 1, 2, 3 amino acid sequences are set forth in SEQ ID NOs: 85, 86, and 87, respectively;
(30) the selected VHH CDRs 1, 2, 3 amino acid sequences are set forth in SEQ ID NOs: 88, 89, and 90, respectively;
(31) the selected VHH CDRs 1, 2, 3 amino acid sequences are set forth in SEQ ID NOs: 91, 92, and 93, respectively;
(32) the selected VHH CDRs 1, 2, 3 amino acid sequences are set forth in SEQ ID NOs: 94, 95, and 96, respectively;
(33) the selected VHH CDRs 1, 2, 3 amino acid sequences are set forth in SEQ ID NOs: 97, 98, and 99, respectively;
(34) the selected VHH CDRs 1, 2, 3 amino acid sequences are set forth in SEQ ID NOs: 100, 101, and 102, respectively;
(35) the selected VHH CDRs 1, 2, 3 amino acid sequences are set forth in SEQ ID NOs: 103, 104, and 105, respectively;
(36) the selected VHH CDRs 1, 2, 3 amino acid sequences are set forth in SEQ ID NOs: 106, 107, and 108, respectively;
(37) the selected VHH CDRs 1, 2, 3 amino acid sequences are set forth in SEQ ID NOs: 109, 110, and 111, respectively;
(38) the selected VHH CDRs 1, 2, 3 amino acid sequences are set forth in SEQ ID NOs: 112, 113, and 114, respectively;
(39) the selected VHH CDRs 1, 2, 3 amino acid sequences are set forth in SEQ ID NOs: 115, 116, and 117, respectively;
(40) the selected VHH CDRs 1, 2, 3 amino acid sequences are set forth in SEQ ID NOs: 118, 119, and 120, respectively;
(41) the selected VHH CDRs 1, 2, 3 amino acid sequences are set forth in SEQ ID NOs: 121, 122, and 123, respectively;
(42) the selected VHH CDRs 1, 2, 3 amino acid sequences are set forth in SEQ ID NOs: 124, 125, and 126, respectively;
(43) the selected VHH CDRs 1, 2, 3 amino acid sequences are set forth in SEQ ID NOs: 127, 128, and 129, respectively;
(44) the selected VHH CDRs 1, 2, 3 amino acid sequences are set forth in SEQ ID NOs: 130, 131, and 132, respectively;
(45) the selected VHH CDRs 1, 2, 3 amino acid sequences are set forth in SEQ ID NOs: 133, 134, and 135, respectively;
(46) the selected VHH CDRs 1, 2, 3 amino acid sequences are set forth in SEQ ID NOs: 136, 137, and 138, respectively;
(47) the selected VHH CDRs 1, 2, 3 amino acid sequences are set forth in SEQ ID NOs: 139, 140, and 141, respectively;
(48) the selected VHH CDRs 1, 2, 3 amino acid sequences are set forth in SEQ ID NOs: 142, 143, and 144, respectively;
(49) the selected VHH CDRs 1, 2, 3 amino acid sequences are set forth in SEQ ID NOs: 145, 146, and 147, respectively;
(50) the selected VHH CDRs 1, 2, 3 amino acid sequences are set forth in SEQ ID NOs: 148, 149, and 150, respectively;
(51) the selected VHH CDRs 1, 2, 3 amino acid sequences are set forth in SEQ ID NOs: 151, 152, and 153, respectively;
(52) the selected VHH CDRs 1, 2, 3 amino acid sequences are set forth in SEQ ID NOs: 154, 155, and 156, respectively;
(53) the selected VHH CDRs 1, 2, 3 amino acid sequences are set forth in SEQ ID NOs: 157, 158, and 159, respectively;
(54) the selected VHH CDRs 1, 2, 3 amino acid sequences are set forth in SEQ ID NOs: 160, 161, and 162, respectively;
(55) the selected VHH CDRs 1, 2, 3 amino acid sequences are set forth in SEQ ID NOs: 163, 164, and 165, respectively;
(56) the selected VHH CDRs 1, 2, 3 amino acid sequences are set forth in SEQ ID NOs: 166, 167, and 168, respectively;
(57) the selected VHH CDRs 1, 2, 3 amino acid sequences are set forth in SEQ ID NOs: 169, 170, and 171, respectively;
(58) the selected VHH CDRs 1, 2, 3 amino acid sequences are set forth in SEQ ID NOs: 172, 173, and 174, respectively;
(59) the selected VHH CDRs 1, 2, 3 amino acid sequences are set forth in SEQ ID NOs: 175, 176, and 177, respectively;
(60) the selected VHH CDRs 1, 2, 3 amino acid sequences are set forth in SEQ ID NOs: 178, 179, and 180, respectively;
(61) the selected VHH CDRs 1, 2, 3 amino acid sequences are set forth in SEQ ID NOs: 181, 182, and 183, respectively;
(62) the selected VHH CDRs 1, 2, 3 amino acid sequences are set forth in SEQ ID NOs: 184, 185, and 186, respectively;
(63) the selected VHH CDRs 1, 2, 3 amino acid sequences are set forth in SEQ ID NOs: 187, 188, and 189, respectively;
(64) the selected VHH CDRs 1, 2, 3 amino acid sequences are set forth in SEQ ID NOs: 190, 191, and 192, respectively;
(65) the selected VHH CDRs 1, 2, 3 amino acid sequences are set forth in SEQ ID NOs: 193, 194, and 195, respectively;
(66) the selected VHH CDRs 1, 2, 3 amino acid sequences are set forth in SEQ ID NOs: 196, 197, and 198, respectively;
(67) the selected VHH CDRs 1, 2, 3 amino acid sequences are set forth in SEQ ID NOs: 199, 200, and 201, respectively;
(68) the selected VHH CDRs 1, 2, 3 amino acid sequences are set forth in SEQ ID NOs: 202, 203, and 204, respectively;
(69) the selected VHH CDRs 1, 2, 3 amino acid sequences are set forth in SEQ ID NOs: 205, 206, and 207, respectively;
(70) the selected VHH CDRs 1, 2, 3 amino acid sequences are set forth in SEQ ID NOs: 208, 209, and 210, respectively;
(71) the selected VHH CDRs 1, 2, 3 amino acid sequences are set forth in SEQ ID NOs: 211, 212, and 213, respectively;
(72) the selected VHH CDRs 1, 2, 3 amino acid sequences are set forth in SEQ ID NOs: 214, 215, and 216, respectively;
(73) the selected VHH CDRs 1, 2, 3 amino acid sequences are set forth in SEQ ID NOs: 217, 218, and 219, respectively;
(74) the selected VHH CDRs 1, 2, 3 amino acid sequences are set forth in SEQ ID NOs: 220, 221, and 222, respectively;
(75) the selected VHH CDRs 1, 2, 3 amino acid sequences are set forth in SEQ ID NOs: 223, 224, and 225, respectively;
(76) the selected VHH CDRs 1, 2, 3 amino acid sequences are set forth in SEQ ID NOs: 226, 227, and 228, respectively;
(77) the selected VHH CDRs 1, 2, 3 amino acid sequences are set forth in SEQ ID NOs: 229, 230, and 231, respectively;
(78) the selected VHH CDRs 1, 2, 3 amino acid sequences are set forth in SEQ ID NOs: 232, 233, and 234, respectively;
(79) the selected VHH CDRs 1, 2, 3 amino acid sequences are set forth in SEQ ID NOs: 235, 236, and 237, respectively;
(80) the selected VHH CDRs 1, 2, 3 amino acid sequences are set forth in SEQ ID NOs: 238, 239, and 240, respectively;
(81) the selected VHH CDRs 1, 2, 3 amino acid sequences are set forth in SEQ ID NOs: 241, 242, and 243, respectively;
(82) the selected VHH CDRs 1, 2, 3 amino acid sequences are set forth in SEQ ID NOs: 244, 245, and 246, respectively;
(83) the selected VHH CDRs 1, 2, 3 amino acid sequences are set forth in SEQ ID NOs: 247, 248, and 249, respectively;
(84) the selected VHH CDRs 1, 2, 3 amino acid sequences are set forth in SEQ ID NOs: 250, 251, and 252, respectively;
(85) the selected VHH CDRs 1, 2, 3 amino acid sequences are set forth in SEQ ID NOs: 253, 254, and 255, respectively;
(86) the selected VHH CDRs 1, 2, 3 amino acid sequences are set forth in SEQ ID NOs: 256, 257, and 258, respectively; and
(87) the selected VHH CDRs 1, 2, 3 amino acid sequences are set forth in SEQ ID NOs: 259, 260, and 261, respectively.

In some embodiments, the VHH comprises CDRs 1, 2, 3 with the amino acid sequences set forth in SEQ ID NOs: 1, 2, and 3, respectively.

In one aspect, provided herein is an antibody or antigen-binding fragment thereof that binds to coronavirus S protein comprising a heavy chain single variable region (VHH) comprising an amino acid sequence that is at least 80% identical to a selected VHH sequence. In some embodiments, the selected VHH sequence is selected from the group consisting of SEQ ID NOS: 262-348. In some embodiments, the VHH comprises the sequence of SEQ ID NO: 262.

In some embodiments, the antibody or antigen-binding fragment specifically binds to coronavirus S protein.

In some embodiments, the antibody or antigen-binding fragment is a humanized antibody or antigen-binding fragment thereof.

In one aspect, provided herein is an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc.

In some embodiments, the antibody or antigen-binding fragment comprises two or more heavy chain single variable domains.

In one aspect, provided herein is a nucleic acid comprising a polynucleotide encoding the antibody or antigen-binding fragment thereof as described herein. In some embodiments, the nucleic acid is cDNA.

In one aspect, provided herein is a vector comprising one or more of the nucleic acids as described herein.

In one aspect, provided herein is a cell comprising the vector as described herein. In some embodiments, the cell is a CHO cell. In one aspect, provided herein is a cell comprising one or more of the nucleic acids as described herein.

In one aspect, provided herein is a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

In one aspect, provided herein is a method of treating a subject having a coronavirus-related disease, the method comprising administering a therapeutically effective amount of a composition comprising the antibody or antigen-binding fragment thereof as described herein to the subject.

In one aspect, provided herein is a method of neutralizing a coronavirus, the method comprising contacting the coronavirus with an effective amount of a composition comprising an antibody or antigen-binding fragment thereof as described herein.

In one aspect, provided herein is a method of blocking internalization of a coronavirus by a cell, the method comprising contacting the coronavirus with an effective amount of a composition comprising the antibody or antigen-binding fragment thereof as described herein.

In one aspect, provided herein is a method of identifying a subject as having a coronavirus disease, the method comprising detecting a sample collected from the subject as having the coronavirus by the antibody or antigen-binding fragment thereof as described herein, thereby identifying the subject as having a coronavirus infection.

In some embodiments, the sample is a blood sample, a saliva sample, a stool sample, or a liquid sample from the respiratory tract of the subject. In some embodiments, the coronavirus is SARS-CoV-2. In some embodiments, the coronavirus is MERS-CoV. In some embodiments, the coronavirus is SARS-CoV.

In one aspect, provided herein is a pharmaceutical composition comprising the antibody or antigen-binding fragment thereof as described herein, and a pharmaceutically acceptable carrier.

In one aspect, provided herein is an antibody or antigen-binding fragment thereof that cross-competes with the antibody or antigen-binding fragment thereof as described herein.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain or VHH). Non-limiting examples of antibody fragments include, e.g., Fab, Fab′, F(ab′)2, and Fv fragments.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to the S protein may be referred to as an S protein-specific antibody or an anti-S protein antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing the workflow of anti-SARS-CoV-2 antibody development. The naïve library was constructed with peripheral blood mononuclear cell (PBMCs) from 65 llamas, and the humanized library was constructed from the naïve VHH library, where the VHH framework was partially humanized and the CDR1, 2, and 3 of VHH were shuffled.

FIG. 2A shows the phylogenetic tree for 69 unique VHH binders.

FIG. 2B is a schematic diagram showing an ELISA assay to assess VHHs binding to recombinant SARS-CoV-2 S proteins.

FIG. 3A is a schematic diagram showing an ACE2 competition assay.

FIG. 3B is a table showing a list of 9 unique S/ACE2 blockers.

FIG. 4 is a table showing ACE2 competition assay results using pairwise combination of the 9 S/ACE2 blockers. (−): >=100%, (+): 80%-100%, and (++): <80% of the signal remaining compared to single VHH additions.

FIG. 5A is a schematic diagram showing receptor-binding domain (RBD) of spike protein binds to ACE2 receptor.

FIG. 5B is a schematic diagram showing structural organization of bi-specific and tri-specific llama VHH nanobody-Fc molecules. The design process utilizes CAAD that optimizes features of nanobody-Fcs.

FIG. 6 shows proposed therapeutic mechanisms of nanobody-Fcs.

FIG. 7 shows an exemplary diagnostic utilization of humanized llama VHHs as single or combinatorial probes.

FIG. 8A shows an epitope binning assay result. Epitope binning of VHH-Fcs were assessed on the Gator (Probe Life) using a 2A-Fc-loaded RBD sensor which quantifies the wavelength shift (indicative of binding signal) over time.

FIG. 8B is a table showing ELISA-based epitope binning assay results. SARS-CoV-2 S1 protein was incubated with 1B-2A-Fc and 3F-Fc, and binding competition was performed with the VHHs followed by the detection of biotinylation. The percent difference from the competing pairs relative to the VHH-Fc alone signal are indicated. The VHH associated percentages for the 1B-2A-Fc group that are over 90% are likely high VHH competitors and partial competitors are over 60%. The VHH associated percentages for the 3F-Fc group that are over 60% are competitors.

FIG. 8C shows two groups of VHHs categorized based on the binding to epitopes on S1 RBD.

FIG. 9A shows ACE2 binding residues on SARS-CoV-2 S1 RBD. ACE2 binding residues on SARS-CoV-2 S1 RBD were determined by Schrodinger BioLuminate® based on the protein-protein interactions of Protein Data Bank (PDB) 6M0J. The residues under arrow symbols are predicted ACE2 interactors.

FIG. 9B shows a SARS-CoV-2 S1 RBD protein sequence and deletion map schematics. The boxed regions in upper panel represent the deletions with deletion map schematics of each deletion mutant in the lower panel.

FIG. 9C shows percentage of FITC positive Expi293 cells expressing SARS-CoV-2 S1 wild type (WT) or mutant proteins (del1-del5). The binding of VHH-Fcs or ACE2 to the Expi293 cells were assessed by flow cytometry following FITC-conjugated secondary antibody treatment. An isotype control antibody and FACS buffer were used as negative controls.

FIG. 9D is a table showing the binding percentage relative to the S1 WT for each VHH-Fc. The binding percentage was calculated in the context of each deletion mutant.

FIG. 9E shows docking models between SARS-CoV-2 S1 RBD and the lead VHHs (1B, 3F, and 2A) generated with Schrodinger BioLuminate® software.

FIG. 10A shows binding of multi-specific, bi-specific, and monoclonal VHH-Fcs to SARS-CoV-2 S1 protein at different concentrations in duplicates using an ELISA method. The binding signal is based on fluorescence, indicated as Relative Fluorescence Units (RFU). Error bars represent standard deviation.

FIG. 10B shows a binding kinetic graph for tri-specific VHH-Fc 1B-3F-2A-Fc.

FIG. 10C shows a binding kinetic graph for tri-specific VHH-Fc 3F-1B-2A-Fc.

FIG. 10D shows blocking of SARS-CoV-2 S/ACE2 interaction by multi-specific, bi-specific, and monoclonal VHH-Fcs at different concentrations in duplicates using an ELISA method. Percent inhibition was calculated based on the blocking signal in RFU for each VHH-Fc treatment. Error bars represent standard deviation.

FIG. 10E is a table showing developability features examining the biophysical and chemical characteristics of VHH-Fcs using DLS (dynamic light scattering), DSF (differential scanning fluorimetry), and SLS (static light scattering).

FIG. 11A shows blocking of SARS-CoV-2 pseudovirus infection by VHH-Fc 3F-1B-2A-Fc, 1B-3F-2A-Fc, and a combination treatment of VHH-Fcs 1B, 3F and 2A. HEK293 ACE2/TMPRSS2 cells were incubated with SARS-CoV-2 pseudovirus and 1:5 serial dilutions of VHH-Fcs, starting at 1000 nM in triplicates. Percent inhibition was calculated based the luminescence signal in RFU for each VHH-Fc treatment. Error bars represent standard error of the mean.

FIG. 11B shows calculated cell death percentage based on cell percentage of VHH-Fc treated cells relative to the isotype control. ADCC function was assessed for the tri-specific VHH-Fcs (3F-1B-2A, 3A-3F-2A and an isotype control antibody) in duplicates. Error bars represent standard deviation.

FIGS. 12A-12C are structure docking model showing how 3F-1B-2A-Fc interacts with SARS-CoV-2 S1 RBD. 3D docking model for SARS-CoV-2 S1 RBD with tri-specific VHH-Fc 3F-1B-2A was generated by BioLuminate®. In the software, the SARS-CoV-2 RBD spike protein trimer (PDB 6X2A) was split into it three monomeric forms (Chain A, B and C). Then, 1B/3F/RBD model structure was aligned with chain A of PDB 6X2A to create Group 1 and 2A/RBD model structure was aligned with chain B of PDB 6X2A to create Group 2. Then, Group 1, Group 2 and chain C were merged to generate the final structure. The S1 RBD/VHH docking structure is represented with a surface structure (FIG. 12A) and ribbon structure (FIG. 12B). The enlarged S1 RBD/VHH docking structure is shown in right (FIG. 12C).

FIG. 13A shows binding of tri-specific VHH-Fcs 3A-3F-2A and 3F-1B-2A to SARS-CoV-2 S1 protein at different concentrations in duplicates using an ELISA method. The binding signal is based on fluorescence, indicated as Relative Fluorescence Units (RFU). Error bars represent standard deviation.

FIG. 13B shows blocking of SARS-CoV-2 S/ACE2 interaction by VHH-Fcs 3A-3F-2A and 3F-1B-2A at different concentrations in duplicates using an ELISA method. Percent inhibition was calculated based the blocking signal in RFU for each VHH-Fc treatment. Error bars represent standard deviation.

FIG. 14 shows a schematic administration strategy of the 3F-1B-2A-Fc anti-SARS-CoV-2 tri-specific antibody (TriAb) in transgenic mice expressing human ACE2. The antibody was administered by intranasal (IN) route or intraperitoneal (IP) route in the treatment groups G2-G4. No antibody was administered in the control group G1. The term “hpi” stands for hour(s) post infection.

FIG. 15 shows the SARS-CoV-2 viral titer in the lungs collected from the control group mice (G1) and treatment group mice (G2-G4) 3 days post SARS-CoV-2 infection.

FIG. 16 shows the relative average body weight of mice in the control group (G1) and treatment groups (G2-G4) as compared to the average initial body weight.

FIG. 17A shows a binding kinetic graph for the 3F-1B-2A-Fc anti-SARS-CoV-2 tri-specific antibody (TriAb) binding to an S protein with wild-type RBD.

FIG. 17B shows a binding kinetic graph for the 3F-1B-2A-Fc anti-SARS-CoV-2 tri-specific antibody (TriAb) binding to an S protein with three mutations in the RBD (K417N/E484K/N501Y), or RBD tri-mut.

FIG. 18 shows blocking of RBD/ACE2 interaction or RBD tri-mut (K417N/E484K/N501Y)/ACE2 interaction by tri-specific 3F-1B-2A-Fc and monoclonal VHH-Fcs (3F+1B+2A) at different concentrations in duplicates using an ELISA method. Percent inhibition was calculated based on the blocking signal in RFU for each VHH-Fc treatment.

FIG. 19 shows a binding kinetic graph determined by bio-layer interferometry (BLI) using tri-specific antibody 3F-1B-2A-Fc that was heated at 45° C. for 2 weeks.

FIG. 20 are tables showing developability features examining the biophysical and chemical characteristics of tri-specific antibody 3F-1B-2A-Fc using DLS (dynamic light scattering), DSF (differential scanning fluorimetry), and SLS (static light scattering). 3F-1B-2A-Fc was kept at 45° C. for 2 weeks or 3 weeks before measurement. Tm stands for melting temperature. Tagg stands for aggregation temperature.

FIG. 21 lists CDR sequences of anti-SARS-CoV-2 S protein antibodies.

FIG. 22 lists amino acid sequences of VHH of anti-SARS-CoV-2 S protein antibodies. Underlined sequences are CDR sequences.

FIG. 23 lists specific combinations of Group 1-Group 2-Group 1 VHHs.

FIG. 24 lists specific combinations of Group 2-Group 1-Group 1 VHHs.

FIG. 25 lists specific combinations of Group 2-Group 2-Group 1 VHHs.

### DETAILED DESCRIPTION

SARS-CoV-2 is a newly emergent coronavirus that causes COVID-19, which has adversely impacted human health and has led to a pandemic. There is an unmet need to develop therapies against SARS-CoV-2 due to its severity and lack of treatment options. A promising approach to combat COVID-19 is through the neutralization of SARS-CoV-2 by therapeutic antibodies.

SARS-CoV2 is a coronavirus that causes the human disease COVID-19, which is contagious and can rapidly spread to cause mild to severe infection, including death (CDC). The spread of this newly emergent virus has reached a pandemic level with a significant public impact on the world, leading to more than 12 million infections and more than a half million deaths worldwide (World Health Organization (WHO)). In addition to threatening human health, COVID-19 has also caused a significant socio-economic impact around the world (United Nations).

Although there are relatively successful diagnostic methods to detect the SARS-CoV-2 infection in humans, there are currently no successful therapies that can stop the infection of the virus. However, recent findings indicate that the small molecule antiviral, Remdesivir (Gilead), inhibits the RNA-dependent RNA polymerase of SARS-CoV-2, which decreases the recovery time in patients with COVID-19, but it most likely cannot completely stop or prevent SARS-CoV-2 infections in humans. Moreover, there are no approved vaccines to prevent SARS-CoV-2 infections in humans, although several groups are currently in the pursuit such vaccines (WHO). Therefore, rapid development of therapeutics and preventative strategies has become an essential and urgent need to fight the COVID-19 pandemic.

The trimeric spike (S) proteins that protrude through the envelope of the SARS-CoV-2 virion mediates virus entry into the host cells by interacting with the ACE2 human receptor. Therefore, a major target for anti-SARS-CoV-2 neutralizing antibodies in development are to block the interaction of SARS-CoV-2 S1 protein with ACE2. In particular, two popular strategies have been employed to discover and develop monoclonal IgG antibodies that can recognize SARS-CoV-2 S1 protein mainly by binding to its receptor binding domain (RBD). The first commonly used method is to clone the antibody V genes from the B cells of surviving COVID-19 patients who have mounted a natural immune response against SARS-CoV-2. This strategy has yielded a number of neutralizing monoclonal antibodies; however, it is important to note that the patients' antibody repertoire condition and the timing of blood sample collection play a critical role in its success. The other well-recognized and classic approach for antibody generation is by immunizing humanized mice. Additionally, the last notable method to generate new SARS-CoV-2 antibodies was developed by screening cross-neutralizing antibodies for the SARS-CoV-2 S1 protein binders from the antibodies that were initially tested or developed to treat SARS by blocking SARS-CoV S/ACE2 or MERS by blocking MERS-CoV S/CD26 interactions. One of the cross-binders is a single domain antibody/nanobody (VHH) generated from SARS-CoV S-immunized llama. Moreover, VHHs against SARS-CoV-2 have also been generated from the llama VHH libraries. The approach of using camelid antibody VHHs is advantageous because the VHH regions are easy to produce, are stable, and are smaller sized, which increases the possibility to target unique epitopes that are not accessible to conventional VH/VL antibodies.

The SARS-CoV-2 virion consists of a helical capsid formed by nucleocapsid (N) proteins bound to the RNA genome, which is enclosed by membrane (M) proteins, envelope (E) proteins and trimeric spike (S) proteins that render them their “corona-like” appearance (See Zhou P, Yang X L, Wang X G, et al. A pneumonia outbreak associated with a new coronavirus of probable bat origin. Nature. 2020 March; 579(7798):270-273). The S protein receptor binding domain (RBD) in the S1 subunit binds to the angiotensin converting enzyme (Angiotensin I Converting Enzyme 2; ACE2) on the cell membranes of type 2 pneumocytes and intestinal epithelial cells. Following binding, the S protein is cleaved by host cell transmembrane serine protease 2 (TMPRSS2), which facilitates subsequent viral entry into host cell (See Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell. 2020 Mar. 4).

Therapeutic antibodies are known to neutralize viral infections via two mechanisms of action, i.e., Fc-independent functions that block capsid/host receptor interaction, and induce virus aggregation, and Fc-dependent functions that cause Fc-FcR interaction to activate immune cells leading to killing of virus (See Klasse P J. Neutralization of Virus Infectivity by Antibodies: Old Problems in New Perspectives. Adv Biol. 2014; 2014). In general, polyclonal antibodies have demonstrated better viral neutralizing ability compared to monoclonal antibodies. However, immunoglobulin isolation from COVID-19 survivors is limited by the lack of availability of plasma from donors. As an alternative therapeutic approach, combinatorial treatment with several monoclonal antibodies is also limited due to high cost of production and potential toxicity. Thus, an approach was employed using humanized llama antibodies that blocks the interaction of SARS-CoV-2 S protein and ACE2, with the goal of rapidly developing high affinity and avidity bi- or tri-specific therapeutic antibodies that neutralize SARS-CoV-2 before it infects cells.

Previous reports have shown that if viruses are bound by low titer therapeutic antibodies with low affinity and avidity, the Fc-FcR interaction might trigger antibody-dependent enhancement (ADE) of virus entry into host cells (See Zellweger R M, Prestwood T R, Shresta S. Enhanced infection of liver sinusoidal endothelial cells in a mouse model of antibody-induced severe dengue disease. Cell Host Microbe. 2010 Feb. 18; 7(2):128-39). Therefore, ADE should be circumvented by developing high titer neutralizing antibodies.

The present disclosure provides a strategy to rapidly identify and generate llama nanobodies (VHH) from naïve and synthetic humanized VHH phage libraries that specifically bind the S1 SARS-CoV-2 spike protein, and block the interaction to the ACE2 human receptor. Computer-aided design was used to construct multi-specific VHH antibodies fused to human IgG1 Fc domains based on the epitope predictions for leading VHHs. The resulting tri-specific VHH-Fc antibodies show more potent S1 binding, S1/ACE2 blocking, and SARS-CoV-2 pseudovirus neutralization than the bi-specific VHH-Fcs or combination of individual monoclonal VHH-Fcs. Furthermore, protein stability analysis of the VHH-Fcs show favorable developability features, which enable them to be quickly and successfully developed into therapeutics against COVID-19.

The present disclosure provides antibodies, antigen-binding fragments thereof that specifically bind to a coronavirus (e.g., SARS-CoV-2, SARS-CoV, or MERS-CoV) S protein. These antibodies or antigen-binding fragments thereof are high titer neutralizing antibodies or antigen-binding fragments thereof. In some embodiments, the antibodies or antigen-binding fragments thereof comprise or consist of one, two, three, four, five or more humanized llama heavy chain single variable domain (VHH).

Particularly, the present disclosure also provides antibodies or antigen-binding fragments with two or more VHHs, which can further provide for at least 10%, 20%, 30%, 40%, 50%, 60%, 70%, 80%, 90%, 1 fold, 2 folds, 5 folds, or 10 folds improvement in the binding affinity and/or avidity to the coronavirus S protein as compared to a similar antibody or antigen-binding fragment thereof with a single VHH.

The disclosure further provides methods of treating COVID-19 using the antibodies or antigen-binding fragments thereof as described herein, and methods of diagnosing COVID-19 using the antibodies or antigen-binding fragments thereof as described herein.

### Heavy Chain Single Variable Domain (VHH) Antibodies

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain IgG is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, or nanobody) is the smallest antigen-binding domain generated by adaptive immune systems. The third Complementarity Determining Region (CDR3) of the variable region of these antibodies has been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into bispecific antibody constructs than constructs containing conventional VH-VL pairs or single domains based on VH domains.

The disclosure provides e.g., anti-coronavirus (e.g., SARS-CoV-2, SARS-CoV, or MERS-CoV) S protein antibodies, the modified antibodies thereof, the chimeric antibodies thereof, and the humanized antibodies thereof.

The CDR sequences for Covid19-E2A3, and Covid19-E2A3 derived antibodies (e.g., humanized antibodies) include CDRs of the VHH domain as set forth in SEQ ID NOs: 1, 2, and 3, respectively.

The CDR sequences for Covid19-E2A6, and Covid19-E2A6 derived antibodies (e.g., humanized antibodies) include CDRs of the VHH domain as set forth in SEQ ID NOs: 4, 5, and 6, respectively.

The CDR sequences for Covid19-E2A8, and Covid19-E2A8 derived antibodies (e.g., humanized antibodies) include CDRs of the VHH domain as set forth in SEQ ID NOs: 7, 8, and 9, respectively.

The CDR sequences for Covid19-E2B3, and Covid19-E2B3 derived antibodies (e.g., humanized antibodies) include CDRs of the VHH domain as set forth in SEQ ID NOs: 10, 11, and 12, respectively.

The CDR sequences for Covid19-E2B7, and Covid19-E2B7 derived antibodies (e.g., humanized antibodies) include CDRs of the VHH domain as set forth in SEQ ID NOs: 13, 14, and 15, respectively.

The CDR sequences for Covid19-E2B10, and Covid19-E2B10 derived antibodies (e.g., humanized antibodies) include CDRs of the VHH domain as set forth in SEQ ID NOs: 16, 17, and 18, respectively.

The CDR sequences for Covid19-E2C6, and Covid19-E2C6 derived antibodies (e.g., humanized antibodies) include CDRs of the VHH domain as set forth in SEQ ID NOs: 19, 20, and 21, respectively.

The CDR sequences for Covid19-E2C7, and Covid19-E2C7 derived antibodies (e.g., humanized antibodies) include CDRs of the VHH domain as set forth in SEQ ID NOs: 22, 23, and 24, respectively.

The CDR sequences for Covid19-E2C9, and Covid19-E2C9 derived antibodies (e.g., humanized antibodies) include CDRs of the VHH domain as set forth in SEQ ID NOs: 25, 26, and 27, respectively.

The CDR sequences for Covid19-E2D4, and Covid19-E2D4 derived antibodies (e.g., humanized antibodies) include CDRs of the VHH domain as set forth in SEQ ID NOs: 28, 29, and 30, respectively.

The CDR sequences for Covid19-E2E2, and Covid19-E2E2 derived antibodies (e.g., humanized antibodies) include CDRs of the VHH domain as set forth in SEQ ID NOs: 31, 32, and 33, respectively.

The CDR sequences for Covid19-E2E3, and Covid19-E2E3 derived antibodies (e.g., humanized antibodies) include CDRs of the VHH domain as set forth in SEQ ID NOs: 34, 35, and 36, respectively.

The CDR sequences for Covid19-E2F3, and Covid19-E2F3 derived antibodies (e.g., humanized antibodies) include CDRs of the VHH domain as set forth in SEQ ID NOs: 37, 38, and 39, respectively.

The CDR sequences for Covid19-E2F6, and Covid19-E2F6 derived antibodies (e.g., humanized antibodies) include CDRs of the VHH domain as set forth in SEQ ID NOs: 40, 41, and 42, respectively.

The CDR sequences for Covid19-E2G8, and Covid19-E2G8 derived antibodies (e.g., humanized antibodies) include CDRs of the VHH domain as set forth in SEQ ID NOs: 43, 44, and 45, respectively.

The CDR sequences for Covid19-E2H2, and Covid19-E2H2 derived antibodies (e.g., humanized antibodies) include CDRs of the VHH domain as set forth in SEQ ID NOs: 46, 47, and 48, respectively.

The CDR sequences for Covid19-E2P2B12, and Covid19-E2P2B12 derived antibodies (e.g., humanized antibodies) include CDRs of the VHH domain as set forth in SEQ ID NOs: 49, 50, and 51, respectively.

The CDR sequences for Covid19-E2P2F11, and Covid19-E2P2F11 derived antibodies (e.g., humanized antibodies) include CDRs of the VHH domain as set forth in SEQ ID NOs: 52, 53, and 54, respectively.

The CDR sequences for Covid19-E2P2G1, and Covid19-E2P2G1 derived antibodies (e.g., humanized antibodies) include CDRs of the VHH domain as set forth in SEQ ID NOs: 55, 56, and 57, respectively.

The CDR sequences for Covid19-E2P2G4, and Covid19-E2P2G4 derived antibodies (e.g., humanized antibodies) include CDRs of the VHH domain as set forth in SEQ ID NOs: 58, 59, and 60, respectively.

The CDR sequences for Covid19-E2P2H4, and Covid19-E2P2H4 derived antibodies (e.g., humanized antibodies) include CDRs of the VHH domain as set forth in SEQ ID NOs: 61, 62, and 63, respectively.

The CDR sequences for Covid19-S1A4, and Covid19-S1A4 derived antibodies (e.g., humanized antibodies) include CDRs of the VHH domain as set forth in SEQ ID NOs: 64, 65, and 66, respectively.

The CDR sequences for Covid19-S1A9, and Covid19-S1A9 derived antibodies (e.g., humanized antibodies) include CDRs of the VHH domain as set forth in SEQ ID NOs: 67, 68, and 69, respectively.

The CDR sequences for Covid19-S1A10, and Covid19-S1A10 derived antibodies (e.g., humanized antibodies) include CDRs of the VHH domain as set forth in SEQ ID NOs: 70, 71, and 72, respectively.

The CDR sequences for Covid19-S1B4, and Covid19-S1B4 derived antibodies (e.g., humanized antibodies) include CDRs of the VHH domain as set forth in SEQ ID NOs: 73, 74, and 75, respectively.

The CDR sequences for Covid19-S1B5, and Covid19-S1B5 derived antibodies (e.g., humanized antibodies) include CDRs of the VHH domain as set forth in SEQ ID NOs: 76, 77, and 78, respectively.

The CDR sequences for Covid19-S1B10, and Covid19-S1B10 derived antibodies (e.g., humanized antibodies) include CDRs of the VHH domain as set forth in SEQ ID NOs: 79, 80, and 81, respectively.

The CDR sequences for Covid19-S1B12, and Covid19-S1B12 derived antibodies (e.g., humanized antibodies) include CDRs of the VHH domain as set forth in SEQ ID NOs: 82, 83, and 84, respectively.

The CDR sequences for Covid19-S1C3, and Covid19-S1C3 derived antibodies (e.g., humanized antibodies) include CDRs of the VHH domain as set forth in SEQ ID NOs: 85, 86, and 87, respectively.

The CDR sequences for Covid19-S1C8, and Covid19-S1C8 derived antibodies (e.g., humanized antibodies) include CDRs of the VHH domain as set forth in SEQ ID NOs: 88, 89, and 90, respectively.

The CDR sequences for Covid19-S1C10, and Covid19-S1C10 derived antibodies (e.g., humanized antibodies) include CDRs of the VHH domain as set forth in SEQ ID NOs: 91, 92, and 93, respectively.

The CDR sequences for Covid19-S1D2, and Covid19-S1D2 derived antibodies (e.g., humanized antibodies) include CDRs of the VHH domain as set forth in SEQ ID NOs: 94, 95, and 96, respectively.

The CDR sequences for Covid19-S1D6, and Covid19-S1D6 derived antibodies (e.g., humanized antibodies) include CDRs of the VHH domain as set forth in SEQ ID NOs: 97, 98, and 99, respectively.

The CDR sequences for Covid19-S1D8, and Covid19-S1D8 derived antibodies (e.g., humanized antibodies) include CDRs of the VHH domain as set forth in SEQ ID NOs: 100, 101, and 102, respectively.

The CDR sequences for Covid19-S1D12, and Covid19-S1D12 derived antibodies (e.g., humanized antibodies) include CDRs of the VHH domain as set forth in SEQ ID NOs: 103, 104, and 105, respectively.

The CDR sequences for Covid19-S1E1, and Covid19-S1E1 derived antibodies (e.g., humanized antibodies) include CDRs of the VHH domain as set forth in SEQ ID NOs: 106, 107, and 108, respectively.

The CDR sequences for Covid19-S1E6, and Covid19-S1E6 derived antibodies (e.g., humanized antibodies) include CDRs of the VHH domain as set forth in SEQ ID NOs: 109, 110, and 111, respectively.

The CDR sequences for Covid19-S1E8, and Covid19-S1E8 derived antibodies (e.g., humanized antibodies) include CDRs of the VHH domain as set forth in SEQ ID NOs: 112, 113, and 114, respectively.

The CDR sequences for Covid19-S1F5, and Covid19-S1F5 derived antibodies (e.g., humanized antibodies) include CDRs of the VHH domain as set forth in SEQ ID NOs: 115, 116, and 117, respectively.

The CDR sequences for Covid19-S1F9, and Covid19-S1F9 derived antibodies (e.g., humanized antibodies) include CDRs of the VHH domain as set forth in SEQ ID NOs: 118, 119, and 120, respectively.

The CDR sequences for Covid19-S1F11, and Covid19-S1F11 derived antibodies (e.g., humanized antibodies) include CDRs of the VHH domain as set forth in SEQ ID NOs: 121, 122, and 123, respectively.

The CDR sequences for Covid19-S1F12, and Covid19-S1F12 derived antibodies (e.g., humanized antibodies) include CDRs of the VHH domain as set forth in SEQ ID NOs: 124, 125, and 126, respectively.

The CDR sequences for Covid19-S1G4, and Covid19-S1G4 derived antibodies (e.g., humanized antibodies) include CDRs of the VHH domain as set forth in SEQ ID NOs: 127, 128, and 129, respectively.

The CDR sequences for Covid19-S1G5, and Covid19-S1G5 derived antibodies (e.g., humanized antibodies) include CDRs of the VHH domain as set forth in SEQ ID NOs: 130, 131, and 132, respectively.

The CDR sequences for Covid19-S1G6, and Covid19-S1G6 derived antibodies (e.g., humanized antibodies) include CDRs of the VHH domain as set forth in SEQ ID NOs: 133, 134, and 135, respectively.

The CDR sequences for Covid19-S1G7, and Covid19-S1G7 derived antibodies (e.g., humanized antibodies) include CDRs of the VHH domain as set forth in SEQ ID NOs: 136, 137, and 138, respectively.

The CDR sequences for Covid19-S1G10, and Covid19-S1G10 derived antibodies (e.g., humanized antibodies) include CDRs of the VHH domain as set forth in SEQ ID NOs: 139, 140, and 141, respectively.

The CDR sequences for Covid19-S1H1, and Covid19-S1H1 derived antibodies (e.g., humanized antibodies) include CDRs of the VHH domain as set forth in SEQ ID NOs: 142, 143, and 144, respectively.

The CDR sequences for Covid19-S1H3, and Covid19-S1H3 derived antibodies (e.g., humanized antibodies) include CDRs of the VHH domain as set forth in SEQ ID NOs: 145, 146, and 147, respectively.

The CDR sequences for Covid19-S1H6, and Covid19-S1H6 derived antibodies (e.g., humanized antibodies) include CDRs of the VHH domain as set forth in SEQ ID NOs: 148, 149, and 150, respectively.

The CDR sequences for Covid19-S1H7, and Covid19-S1H7 derived antibodies (e.g., humanized antibodies) include CDRs of the VHH domain as set forth in SEQ ID NOs: 151, 152, and 153, respectively.

The CDR sequences for Covid19-S1H8, and Covid19-S1H8 derived antibodies (e.g., humanized antibodies) include CDRs of the VHH domain as set forth in SEQ ID NOs: 154, 155, and 156, respectively.

The CDR sequences for Covid19-S1P2A10, and Covid19-S1P2A10 derived antibodies (e.g., humanized antibodies) include CDRs of the VHH domain as set forth in SEQ ID NOs: 157, 158, and 159, respectively.

The CDR sequences for Covid19-S1P2A12, and Covid19-S1P2A12 derived antibodies (e.g., humanized antibodies) include CDRs of the VHH domain as set forth in SEQ ID NOs: 160, 161, and 162, respectively.

The CDR sequences for Covid19-S1P2C8, and Covid19-S1P2C8 derived antibodies (e.g., humanized antibodies) include CDRs of the VHH domain as set forth in SEQ ID NOs: 163, 164, and 165, respectively.

The CDR sequences for Covid19-S1P2F5, and Covid19-S1P2F5 derived antibodies (e.g., humanized antibodies) include CDRs of the VHH domain as set forth in SEQ ID NOs: 166, 167, and 168, respectively.

The CDR sequences for Covid19-S1P2F12, and Covid19-S1P2F12 derived antibodies (e.g., humanized antibodies) include CDRs of the VHH domain as set forth in SEQ ID NOs: 169, 170, and 171, respectively.

The CDR sequences for Covid19-S1P2H4, and Covid19-S1P2H4 derived antibodies (e.g., humanized antibodies) include CDRs of the VHH domain as set forth in SEQ ID NOs: 172, 173, and 174, respectively.

The CDR sequences for Covid19-S1P2H5, and Covid19-S1P2H5 derived antibodies (e.g., humanized antibodies) include CDRs of the VHH domain as set forth in SEQ ID NOs: 175, 176, and 177, respectively.

The CDR sequences for Covid19-S1P2H6, and Covid19-S1P2H6 derived antibodies (e.g., humanized antibodies) include CDRs of the VHH domain as set forth in SEQ ID NOs: 178, 179, and 180, respectively.

The CDR sequences for Covid19-S1P2H9, and Covid19-S1P2H9 derived antibodies (e.g., humanized antibodies) include CDRs of the VHH domain as set forth in SEQ ID NOs: 181, 182, and 183, respectively.

The CDR sequences for Covid19-S1P2H10, and Covid19-S1P2H10 derived antibodies (e.g., humanized antibodies) include CDRs of the VHH domain as set forth in SEQ ID NOs: 184, 185, and 186, respectively.

The CDR sequences for Covid19-S2A3, and Covid19-S2A3 derived antibodies (e.g., humanized antibodies) include CDRs of the VHH domain as set forth in SEQ ID NOs: 187, 188, and 189, respectively.

The CDR sequences for Covid19-1B6, and Covid19-1B6 derived antibodies (e.g., humanized antibodies) include CDRs of the VHH domain as set forth in SEQ ID NOs: 190, 191, and 192, respectively.

The CDR sequences for Covid19-2A4, and Covid19-2A4 derived antibodies (e.g., humanized antibodies) include CDRs of the VHH domain as set forth in SEQ ID NOs: 193, 194, and 195, respectively.

The CDR sequences for Covid19-3A11, and Covid19-3A11 derived antibodies (e.g., humanized antibodies) include CDRs of the VHH domain as set forth in SEQ ID NOs: 196, 197, and 198, respectively.

The CDR sequences for Covid19-3F2, and Covid19-3F2 derived antibodies (e.g., humanized antibodies) include CDRs of the VHH domain as set forth in SEQ ID NOs: 199, 200, and 201, respectively.

The CDR sequences for Covid19-3F10, and Covid19-3F10 derived antibodies (e.g., humanized antibodies) include CDRs of the VHH domain as set forth in SEQ ID NOs: 202, 203, and 204, respectively.

The CDR sequences for 1D7, and 1D7 derived antibodies (e.g., humanized antibodies) include CDRs of the VHH domain as set forth in SEQ ID NOs: 205, 206, and 207, respectively.

The CDR sequences for 1C11, and 1C11 derived antibodies (e.g., humanized antibodies) include CDRs of the VHH domain as set forth in SEQ ID NOs: 208, 209, and 210, respectively.

The CDR sequences for 1F12, and 1F12 derived antibodies (e.g., humanized antibodies) include CDRs of the VHH domain as set forth in SEQ ID NOs: 211, 212, and 213, respectively.

The CDR sequences for 2B4, and 2B4 derived antibodies (e.g., humanized antibodies) include CDRs of the VHH domain as set forth in SEQ ID NOs: 214, 215, and 216, respectively.

The CDR sequences for 2E8, and 2E8 derived antibodies (e.g., humanized antibodies) include CDRs of the VHH domain as set forth in SEQ ID NOs: 217, 218, and 219, respectively.

The CDR sequences for 3A4, and 3A4 derived antibodies (e.g., humanized antibodies) include CDRs of the VHH domain as set forth in SEQ ID NOs: 220, 221, and 222, respectively.

The CDR sequences for 3G7, and 3G7 derived antibodies (e.g., humanized antibodies) include CDRs of the VHH domain as set forth in SEQ ID NOs: 223, 224, and 225, respectively.

The CDR sequences for 3B11, and 3B11 derived antibodies (e.g., humanized antibodies) include CDRs of the VHH domain as set forth in SEQ ID NOs: 226, 227, and 228, respectively.

The CDR sequences for 3B12, and 3B12 derived antibodies (e.g., humanized antibodies) include CDRs of the VHH domain as set forth in SEQ ID NOs: 229, 230, and 231, respectively.

The CDR sequences for 4A9, and 4A9 derived antibodies (e.g., humanized antibodies) include CDRs of the VHH domain as set forth in SEQ ID NOs: 232, 233, and 234, respectively.

The CDR sequences for 4F7, and 4F7 derived antibodies (e.g., humanized antibodies) include CDRs of the VHH domain as set forth in SEQ ID NOs: 235, 236, and 237, respectively.

The CDR sequences for 4C12, and 4C12 derived antibodies (e.g., humanized antibodies) include CDRs of the VHH domain as set forth in SEQ ID NOs: 238, 239, and 240, respectively.

The CDR sequences for 4F12, and 4F12 derived antibodies (e.g., humanized antibodies) include CDRs of the VHH domain as set forth in SEQ ID NOs: 241, 242, and 243, respectively.

The CDR sequences for 1F11_B, and 1F11_B derived antibodies (e.g., humanized antibodies) include CDRs of the VHH domain as set forth in SEQ ID NOs: 244, 245, and 246, respectively.

The CDR sequences for 1F12_B, and 1F12_B derived antibodies (e.g., humanized antibodies) include CDRs of the VHH domain as set forth in SEQ ID NOs: 247, 248, and 249, respectively.

The CDR sequences for 1G12_B, and 1G12_B derived antibodies (e.g., humanized antibodies) include CDRs of the VHH domain as set forth in SEQ ID NOs: 250, 251, and 252, respectively.

The CDR sequences for 2A4_B, and 2A4_B derived antibodies (e.g., humanized antibodies) include CDRs of the VHH domain as set forth in SEQ ID NOs: 253, 254, and 255, respectively.

The CDR sequences for 2A5_B, and 2A5_B derived antibodies (e.g., humanized antibodies) include CDRs of the VHH domain as set forth in SEQ ID NOs: 256, 257, and 258, respectively.

The CDR sequences for 2D12_B, and 2D12_B derived antibodies (e.g., humanized antibodies) include CDRs of the VHH domain as set forth in SEQ ID NOs: 259, 260, and 261, respectively.

The amino acid sequence for the VHH domain of Covid19-E2A3 antibody is set forth in SEQ ID NO: 262.

The amino acid sequence for the VHH domain of Covid19-E2A6 antibody is set forth in SEQ ID NO: 263.

The amino acid sequence for the VHH domain of Covid19-E2A8 antibody is set forth in SEQ ID NO: 264.

The amino acid sequence for the VHH domain of Covid19-E2B3 antibody is set forth in SEQ ID NO: 265.

The amino acid sequence for the VHH domain of Covid19-E2B7 antibody is set forth in SEQ ID NO: 266.

The amino acid sequence for the VHH domain of Covid19-E2B10 antibody is set forth in SEQ ID NO: 267.

The amino acid sequence for the VHH domain of Covid19-E2C6 antibody is set forth in SEQ ID NO: 268.

The amino acid sequence for the VHH domain of Covid19-E2C7 antibody is set forth in SEQ ID NO: 269.

The amino acid sequence for the VHH domain of Covid19-E2C9 antibody is set forth in SEQ ID NO: 270.

The amino acid sequence for the VHH domain of Covid19-E2D4 antibody is set forth in SEQ ID NO: 271.

The amino acid sequence for the VHH domain of Covid19-E2E2 antibody is set forth in SEQ ID NO: 272.

The amino acid sequence for the VHH domain of Covid19-E2E3 antibody is set forth in SEQ ID NO: 273.

The amino acid sequence for the VHH domain of Covid19-E2F3 antibody is set forth in SEQ ID NO: 274.

The amino acid sequence for the VHH domain of Covid19-E2F6 antibody is set forth in SEQ ID NO: 275.

The amino acid sequence for the VHH domain of Covid19-E2G8 antibody is set forth in SEQ ID NO: 276.

The amino acid sequence for the VHH domain of Covid19-E2H2 antibody is set forth in SEQ ID NO: 277.

The amino acid sequence for the VHH domain of Covid19-E2P2B12 antibody is set forth in SEQ ID NO: 278.

The amino acid sequence for the VHH domain of Covid19-E2P2F11 antibody is set forth in SEQ ID NO: 279.

The amino acid sequence for the VHH domain of Covid19-E2P2G1 antibody is set forth in SEQ ID NO: 280.

The amino acid sequence for the VHH domain of Covid19-E2P2G4 antibody is set forth in SEQ ID NO: 281.

The amino acid sequence for the VHH domain of Covid19-E2P2H4 antibody is set forth in SEQ ID NO: 282.

The amino acid sequence for the VHH domain of Covid19-S1A4 antibody is set forth in SEQ ID NO: 283.

The amino acid sequence for the VHH domain of Covid19-S1A9 antibody is set forth in SEQ ID NO: 284.

The amino acid sequence for the VHH domain of Covid19-S1A10 antibody is set forth in SEQ ID NO: 285.

The amino acid sequence for the VHH domain of Covid19-S1B4 antibody is set forth in SEQ ID NO: 286.

The amino acid sequence for the VHH domain of Covid19-S1B5 antibody is set forth in SEQ ID NO: 287.

The amino acid sequence for the VHH domain of Covid19-S1B10 antibody is set forth in SEQ ID NO: 288.

The amino acid sequence for the VHH domain of Covid19-S1B12 antibody is set forth in SEQ ID NO: 289.

The amino acid sequence for the VHH domain of Covid19-S1C3 antibody is set forth in SEQ ID NO: 290.

The amino acid sequence for the VHH domain of Covid19-S1C8 antibody is set forth in SEQ ID NO: 291.

The amino acid sequence for the VHH domain of Covid19-S1C10 antibody is set forth in SEQ ID NO: 292.

The amino acid sequence for the VHH domain of Covid19-S1D2 antibody is set forth in SEQ ID NO: 293.

The amino acid sequence for the VHH domain of Covid19-S1D6 antibody is set forth in SEQ ID NO: 294.

The amino acid sequence for the VHH domain of Covid19-S1D8 antibody is set forth in SEQ ID NO: 295.

The amino acid sequence for the VHH domain of Covid19-S1D12 antibody is set forth in SEQ ID NO: 296.

The amino acid sequence for the VHH domain of Covid19-S1E1 antibody is set forth in SEQ ID NO: 297.

The amino acid sequence for the VHH domain of Covid19-S1E6 antibody is set forth in SEQ ID NO: 298.

The amino acid sequence for the VHH domain of Covid19-S1E8 antibody is set forth in SEQ ID NO: 299.

The amino acid sequence for the VHH domain of Covid19-S1F5 antibody is set forth in SEQ ID NO: 300.

The amino acid sequence for the VHH domain of Covid19-S1F9 antibody is set forth in SEQ ID NO: 301.

The amino acid sequence for the VHH domain of Covid19-S1F11 antibody is set forth in SEQ ID NO: 302.

The amino acid sequence for the VHH domain of Covid19-S1F12 antibody is set forth in SEQ ID NO: 303.

The amino acid sequence for the VHH domain of Covid19-S1G4 antibody is set forth in SEQ ID NO: 304.

The amino acid sequence for the VHH domain of Covid19-S1G5 antibody is set forth in SEQ ID NO: 305.

The amino acid sequence for the VHH domain of Covid19-S1G6 antibody is set forth in SEQ ID NO: 306.

The amino acid sequence for the VHH domain of Covid19-S1G7 antibody is set forth in SEQ ID NO: 307.

The amino acid sequence for the VHH domain of Covid19-S1G10 antibody is set forth in SEQ ID NO: 308.

The amino acid sequence for the VHH domain of Covid19-S1H1 antibody is set forth in SEQ ID NO: 309.

The amino acid sequence for the VHH domain of Covid19-S1H3 antibody is set forth in SEQ ID NO: 310.

The amino acid sequence for the VHH domain of Covid19-S1H6 antibody is set forth in SEQ ID NO: 311.

The amino acid sequence for the VHH domain of Covid19-S1H7 antibody is set forth in SEQ ID NO: 312.

The amino acid sequence for the VHH domain of Covid19-S1H8 antibody is set forth in SEQ ID NO: 313.

The amino acid sequence for the VHH domain of Covid19-S1P2A10 antibody is set forth in SEQ ID NO: 314.

The amino acid sequence for the VHH domain of Covid19-S1P2A12 antibody is set forth in SEQ ID NO: 315.

The amino acid sequence for the VHH domain of Covid19-S1P2C8 antibody is set forth in SEQ ID NO: 316.

The amino acid sequence for the VHH domain of Covid19-S1P2F5 antibody is set forth in SEQ ID NO: 317.

The amino acid sequence for the VHH domain of Covid19-S1P2F12 antibody is set forth in SEQ ID NO: 318.

The amino acid sequence for the VHH domain of Covid19-S1P2H4 antibody is set forth in SEQ ID NO: 319.

The amino acid sequence for the VHH domain of Covid19-S1P2H5 antibody is set forth in SEQ ID NO: 320.

The amino acid sequence for the VHH domain of Covid19-S1P2H6 antibody is set forth in SEQ ID NO: 321.

The amino acid sequence for the VHH domain of Covid19-S1P2H9 antibody is set forth in SEQ ID NO: 322.

The amino acid sequence for the VHH domain of Covid19-S1P2H10 antibody is set forth in SEQ ID NO: 323.

The amino acid sequence for the VHH domain of Covid19-S2A3 antibody is set forth in SEQ ID NO: 324.

The amino acid sequence for the VHH domain of Covid19-1B6 antibody is set forth in SEQ ID NO: 325.

The amino acid sequence for the VHH domain of Covid19-2A4 antibody is set forth in SEQ ID NO: 326.

The amino acid sequence for the VHH domain of Covid19-3A11 antibody is set forth in SEQ ID NO: 327.

The amino acid sequence for the VHH domain of Covid19-3F2 antibody is set forth in SEQ ID NO: 328.

The amino acid sequence for the VHH domain of Covid19-3F10 antibody is set forth in SEQ ID NO: 329.

The amino acid sequence for the VHH domain of 1D7 antibody is set forth in SEQ ID NO: 330.

The amino acid sequence for the VHH domain of 1C11 antibody is set forth in SEQ ID NO: 331.

The amino acid sequence for the VHH domain of 1F12 antibody is set forth in SEQ ID NO: 332.

The amino acid sequence for the VHH domain of 2B4 antibody is set forth in SEQ ID NO: 333.

The amino acid sequence for the VHH domain of 2E8 antibody is set forth in SEQ ID NO: 334.

The amino acid sequence for the VHH domain of 3A4 antibody is set forth in SEQ ID NO: 335.

The amino acid sequence for the VHH domain of 3G7 antibody is set forth in SEQ ID NO: 336.

The amino acid sequence for the VHH domain of 3B11 antibody is set forth in SEQ ID NO: 337.

The amino acid sequence for the VHH domain of 3B12 antibody is set forth in SEQ ID NO: 338.

The amino acid sequence for the VHH domain of 4A9 antibody is set forth in SEQ ID NO: 339.

The amino acid sequence for the VHH domain of 4F7 antibody is set forth in SEQ ID NO: 340.

The amino acid sequence for the VHH domain of 4C12 antibody is set forth in SEQ ID NO: 341.

The amino acid sequence for the VHH domain of 4F12 antibody is set forth in SEQ ID NO: 342.

The amino acid sequence for the VHH domain of 1F11_B antibody is set forth in SEQ ID NO: 343.

The amino acid sequence for the VHH domain of 1F12_B antibody is set forth in SEQ ID NO: 344.

The amino acid sequence for the VHH domain of 1G12_B antibody is set forth in SEQ ID NO: 345.

The amino acid sequence for the VHH domain of 2A4_B antibody is set forth in SEQ ID NO: 346.

The amino acid sequence for the VHH domain of 2A5_B antibody is set forth in SEQ ID NO: 347.

The amino acid sequence for the VHH domain of 2D12_B antibody is set forth in SEQ ID NO: 348.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOS: 262-348.

Furthermore, in some embodiments, the antibodies or antigen-binding fragments thereof described herein can also contain one, two, or three VHH domain CDRs selected from the group of SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24, SEQ ID NOs: 25-27, SEQ ID NOs: 28-30, SEQ ID NOs: 31-33, SEQ ID NOs: 34-36, SEQ ID NOs: 37-39, SEQ ID NOs: 40-42, SEQ ID NOs: 43-45, SEQ ID NOs: 46-48, SEQ ID NOs: 49-51, SEQ ID NOs: 52-54, SEQ ID NOs: 55-57, SEQ ID NOs: 58-60, SEQ ID NOs: 61-63, SEQ ID NOs: 64-66, SEQ ID NOs: 67-69, SEQ ID NOs: 70-72, SEQ ID NOs: 73-75, SEQ ID NOs: 76-78, SEQ ID NOs: 79-81, SEQ ID NOs: 82-84, SEQ ID NOs: 85-87, SEQ ID NOs: 88-90, SEQ ID NOs: 91-93, SEQ ID NOs: 94-96, SEQ ID NOs: 97-99, SEQ ID NOs: 100-102, SEQ ID NOs: 103-105, SEQ ID NOs: 106-108, SEQ ID NOs: 109-111, SEQ ID NOs: 112-114, SEQ ID NOs: 115-117, SEQ ID NOs: 118-120, SEQ ID NOs: 121-123, SEQ ID NOs: 124-126, SEQ ID NOs: 127-129, SEQ ID NOs: 130-132, SEQ ID NOs: 133-135, SEQ ID NOs: 136-138, SEQ ID NOs: 139-141, SEQ ID NOs: 142-144, SEQ ID NOs: 145-147, SEQ ID NOs: 148-150, SEQ ID NOs: 151-153, SEQ ID NOs: 154-156, SEQ ID NOs: 157-159, SEQ ID NOs: 160-162, SEQ ID NOs: 163-165, SEQ ID NOs: 166-168, SEQ ID NOs: 169-171, SEQ ID NOs: 172-174, SEQ ID NOs: 175-177, SEQ ID NOs: 178-180, SEQ ID NOs: 181-183, SEQ ID NOs: 184-186, SEQ ID NOs: 187-189, SEQ ID NOs: 190-192, SEQ ID NOs: 193-195, SEQ ID NOs: 196-198, SEQ ID NOs: 199-201, SEQ ID NOs: 202-204, SEQ ID NOs: 205-207, SEQ ID NOs: 208-210, SEQ ID NOs: 211-213, SEQ ID NOs: 214-216, SEQ ID NOs: 217-219, SEQ ID NOs: 220-222, SEQ ID NOs: 223-225, SEQ ID NOs: 226-228, SEQ ID NOs: 229-231, SEQ ID NOs: 232-234, SEQ ID NOs: 235-237, SEQ ID NOs: 238-240, SEQ ID NOs: 241-243, SEQ ID NOs: 244-246, SEQ ID NOs: 247-249, SEQ ID NOs: 250-252, SEQ ID NOs: 253-255, SEQ ID NOs: 256-258, and SEQ ID NOs: 259-261.

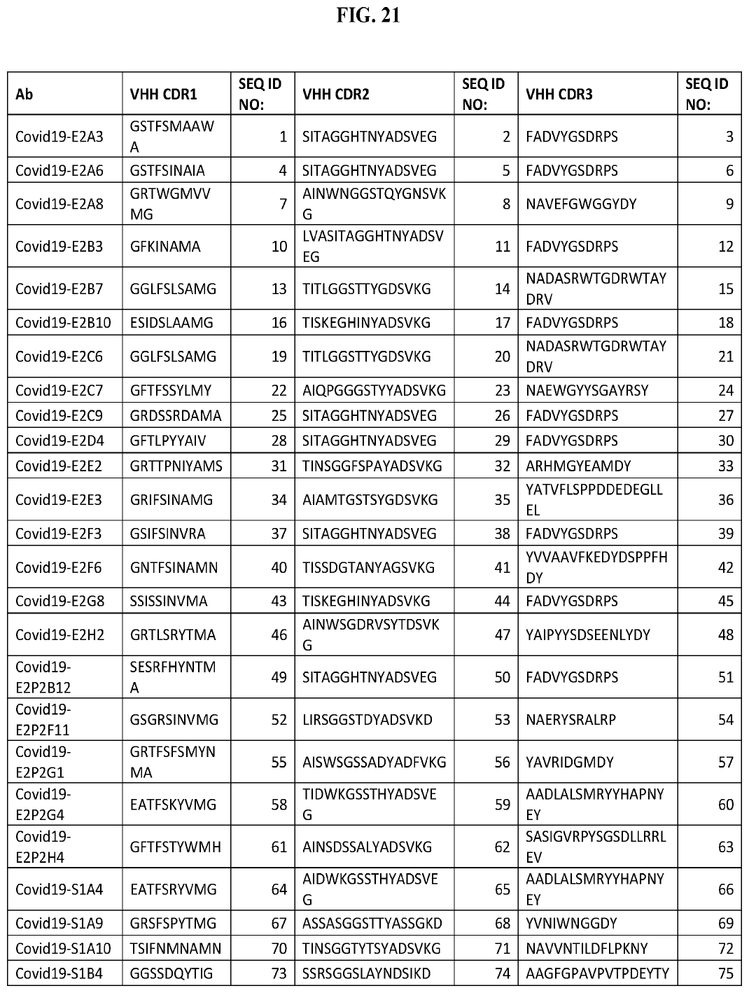

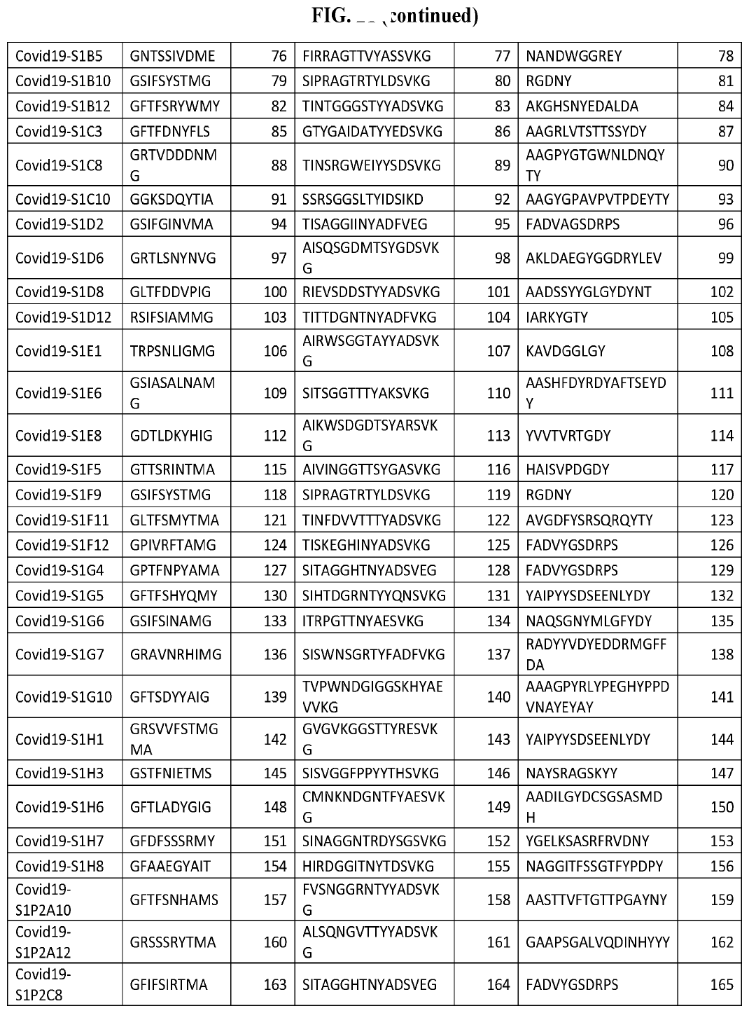

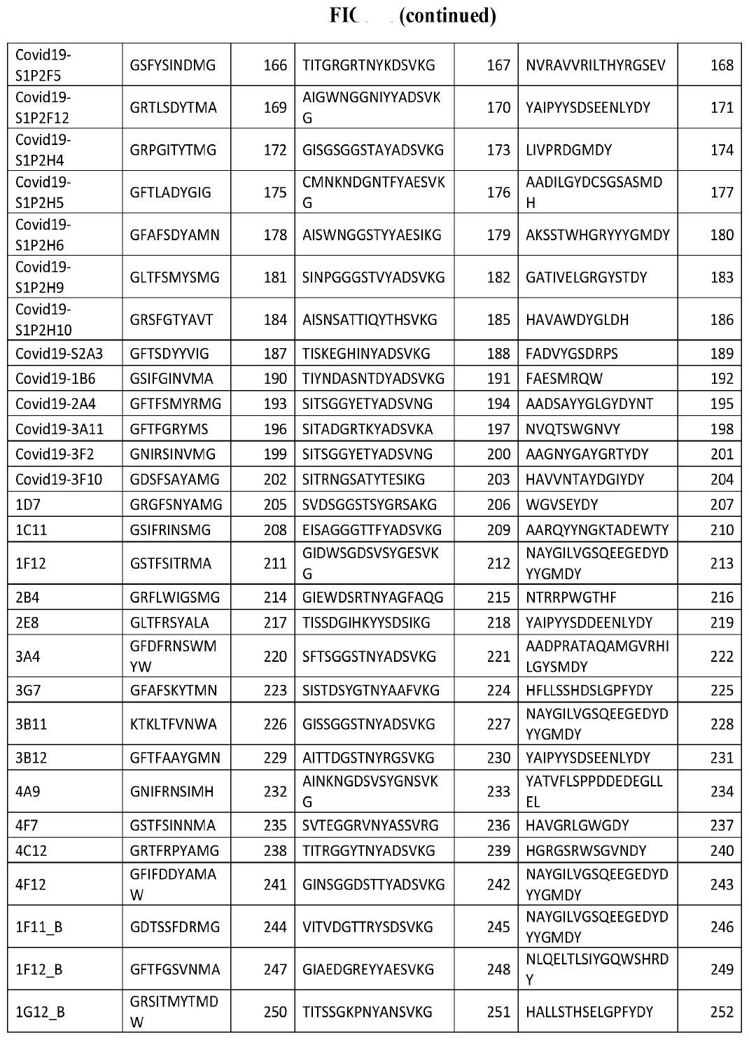

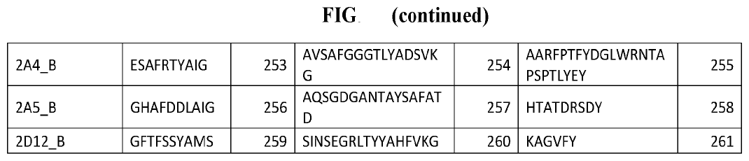

In some embodiments, the antibodies can have a heavy chain single variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 21.

In some embodiments, the antibody or an antigen-binding fragment described herein can contain a heavy chain single variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 21.

In some embodiments, the antibody or an antigen-binding fragment described herein can contain a heavy chain single variable domain (VHH) containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on a combination numbering scheme.

The disclosure also provides antibodies or antigen-binding fragments thereof that bind to a coronavirus (e.g., SARS-CoV-2, SARS-CoV, or MERS-CoV) S protein. The antibodies or antigen-binding fragments thereof contain a heavy chain single variable region (VHH) comprising or consisting of an amino acid sequence that is at least 80%, 85%, 90%, or 95% identical to a selected VHH sequence. In some embodiments, the selected VHH sequence is SEQ ID NO: 262. In some embodiments, the selected VHH sequence is SEQ ID NO: 263. In some embodiments, the selected VHH sequence is SEQ ID NO: 264. In some embodiments, the selected VHH sequence is SEQ ID NO: 265. In some embodiments, the selected VHH sequence is SEQ ID NO: 266. In some embodiments, the selected VHH sequence is SEQ ID NO: 267. In some embodiments, the selected VHH sequence is SEQ ID NO: 268. In some embodiments, the selected VHH sequence is SEQ ID NO: 269. In some embodiments, the selected VHH sequence is SEQ ID NO: 270. In some embodiments, the selected VHH sequence is SEQ ID NO: 271. In some embodiments, the selected VHH sequence is SEQ ID NO: 272. In some embodiments, the selected VHH sequence is SEQ ID NO: 273. In some embodiments, the selected VHH sequence is SEQ ID NO: 274. In some embodiments, the selected VHH sequence is SEQ ID NO: 275. In some embodiments, the selected VHH sequence is SEQ ID NO: 276. In some embodiments, the selected VHH sequence is SEQ ID NO: 277. In some embodiments, the selected VHH sequence is SEQ ID NO: 278. In some embodiments, the selected VHH sequence is SEQ ID NO: 279. In some embodiments, the selected VHH sequence is SEQ ID NO: 280. In some embodiments, the selected VHH sequence is SEQ ID NO: 281. In some embodiments, the selected VHH sequence is SEQ ID NO: 282. In some embodiments, the selected VHH sequence is SEQ ID NO: 283. In some embodiments, the selected VHH sequence is SEQ ID NO: 284. In some embodiments, the selected VHH sequence is SEQ ID NO: 285. In some embodiments, the selected VHH sequence is SEQ ID NO: 286. In some embodiments, the selected VHH sequence is SEQ ID NO: 287. In some embodiments, the selected VHH sequence is SEQ ID NO: 288. In some embodiments, the selected VHH sequence is SEQ ID NO: 289. In some embodiments, the selected VHH sequence is SEQ ID NO: 290. In some embodiments, the selected VHH sequence is SEQ ID NO: 291. In some embodiments, the selected VHH sequence is SEQ ID NO: 292. In some embodiments, the selected VHH sequence is SEQ ID NO: 293. In some embodiments, the selected VHH sequence is SEQ ID NO: 294. In some embodiments, the selected VHH sequence is SEQ ID NO: 295. In some embodiments, the selected VHH sequence is SEQ ID NO: 296. In some embodiments, the selected VHH sequence is SEQ ID NO: 297. In some embodiments, the selected VHH sequence is SEQ ID NO: 298. In some embodiments, the selected VHH sequence is SEQ ID NO: 299. In some embodiments, the selected VHH sequence is SEQ ID NO: 300. In some embodiments, the selected VHH sequence is SEQ ID NO: 301. In some embodiments, the selected VHH sequence is SEQ ID NO: 302. In some embodiments, the selected VHH sequence is SEQ ID NO: 303. In some embodiments, the selected VHH sequence is SEQ ID NO: 304. In some embodiments, the selected VHH sequence is SEQ ID NO: 305. In some embodiments, the selected VHH sequence is SEQ ID NO: 306. In some embodiments, the selected VHH sequence is SEQ ID NO: 307. In some embodiments, the selected VHH sequence is SEQ ID NO: 308. In some embodiments, the selected VHH sequence is SEQ ID NO: 309. In some embodiments, the selected VHH sequence is SEQ ID NO: 310. In some embodiments, the selected VHH sequence is SEQ ID NO: 311. In some embodiments, the selected VHH sequence is SEQ ID NO: 312. In some embodiments, the selected VHH sequence is SEQ ID NO: 313. In some embodiments, the selected VHH sequence is SEQ ID NO: 314. In some embodiments, the selected VHH sequence is SEQ ID NO: 315. In some embodiments, the selected VHH sequence is SEQ ID NO: 316. In some embodiments, the selected VHH sequence is SEQ ID NO: 317. In some embodiments, the selected VHH sequence is SEQ ID NO: 318. In some embodiments, the selected VHH sequence is SEQ ID NO: 319. In some embodiments, the selected VHH sequence is SEQ ID NO: 320. In some embodiments, the selected VHH sequence is SEQ ID NO: 321. In some embodiments, the selected VHH sequence is SEQ ID NO: 322. In some embodiments, the selected VHH sequence is SEQ ID NO: 323. In some embodiments, the selected VHH sequence is SEQ ID NO: 324. In some embodiments, the selected VHH sequence is SEQ ID NO: 325. In some embodiments, the selected VHH sequence is SEQ ID NO: 326. In some embodiments, the selected VHH sequence is SEQ ID NO: 327. In some embodiments, the selected VHH sequence is SEQ ID NO: 328. In some embodiments, the selected VHH sequence is SEQ ID NO: 329. In some embodiments, the selected VHH sequence is SEQ ID NO: 330. In some embodiments, the selected VHH sequence is SEQ ID NO: 331. In some embodiments, the selected VHH sequence is SEQ ID NO: 332. In some embodiments, the selected VHH sequence is SEQ ID NO: 333. In some embodiments, the selected VHH sequence is SEQ ID NO: 334. In some embodiments, the selected VHH sequence is SEQ ID NO: 335. In some embodiments, the selected VHH sequence is SEQ ID NO: 336. In some embodiments, the selected VHH sequence is SEQ ID NO: 337. In some embodiments, the selected VHH sequence is SEQ ID NO: 338. In some embodiments, the selected VHH sequence is SEQ ID NO: 339. In some embodiments, the selected VHH sequence is SEQ ID NO: 340. In some embodiments, the selected VHH sequence is SEQ ID NO: 341. In some embodiments, the selected VHH sequence is SEQ ID NO: 342. In some embodiments, the selected VHH sequence is SEQ ID NO: 343. In some embodiments, the selected VHH sequence is SEQ ID NO: 344. In some embodiments, the selected VHH sequence is SEQ ID NO: 345. In some embodiments, the selected VHH sequence is SEQ ID NO: 346. In some embodiments, the selected VHH sequence is SEQ ID NO: 347. In some embodiments, the selected VHH sequence is SEQ ID NO: 348.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

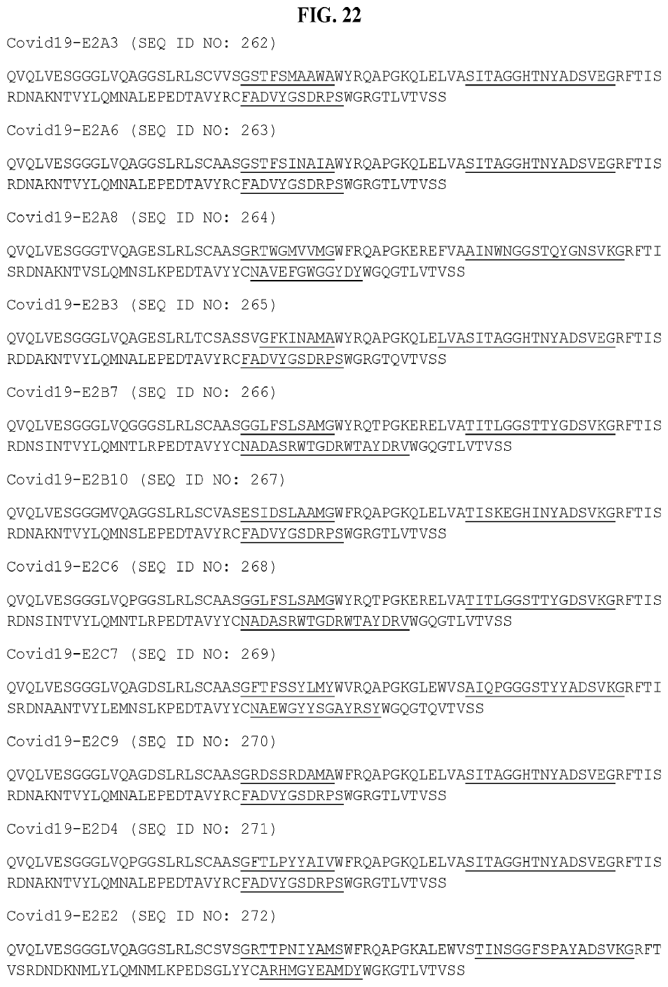

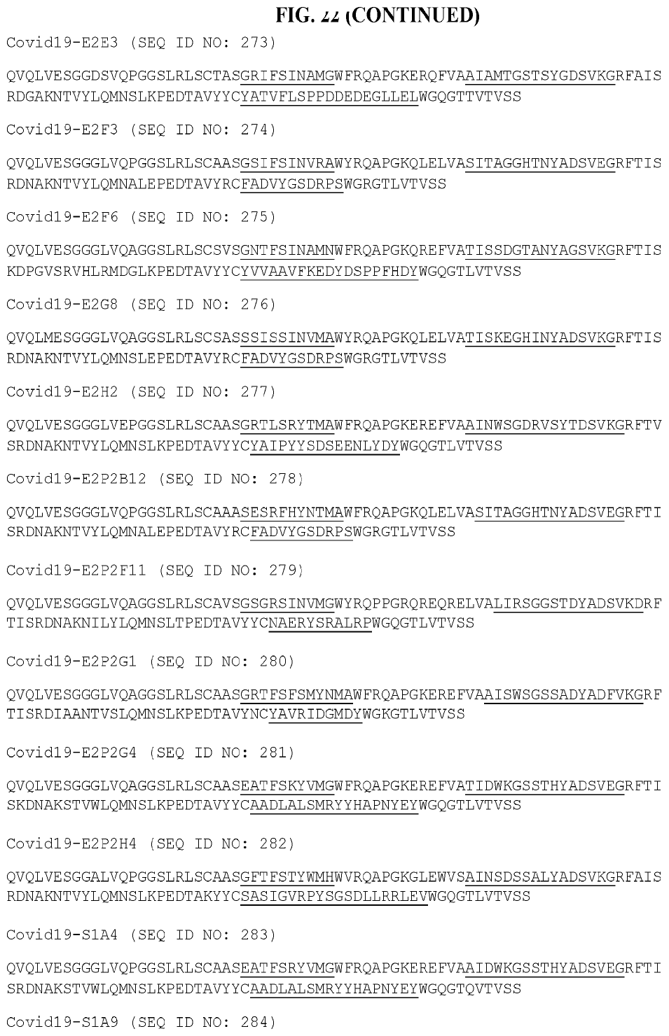

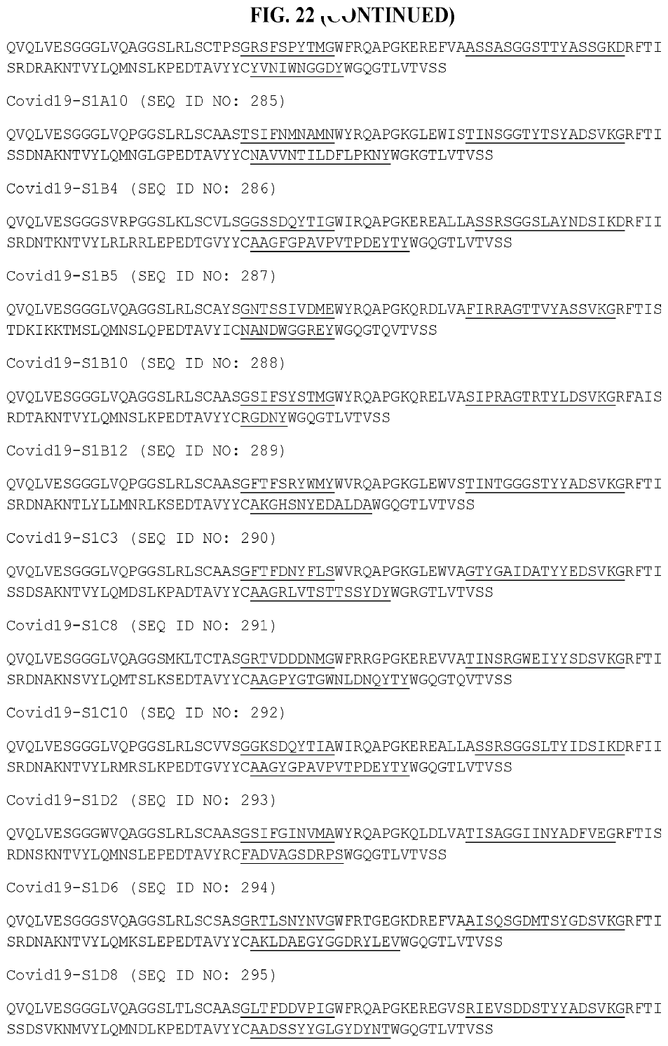

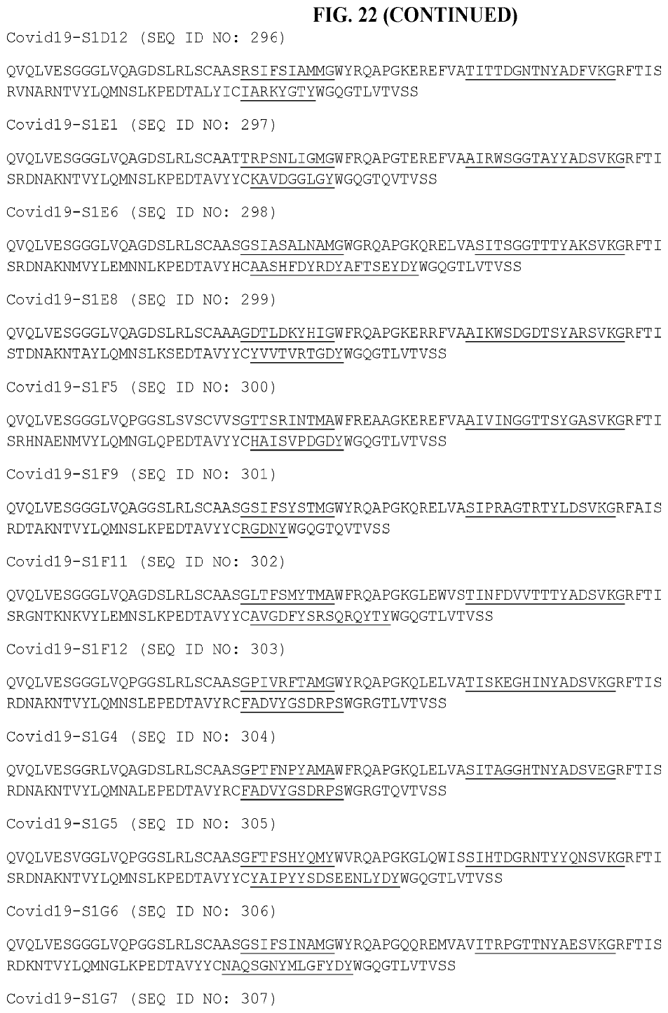

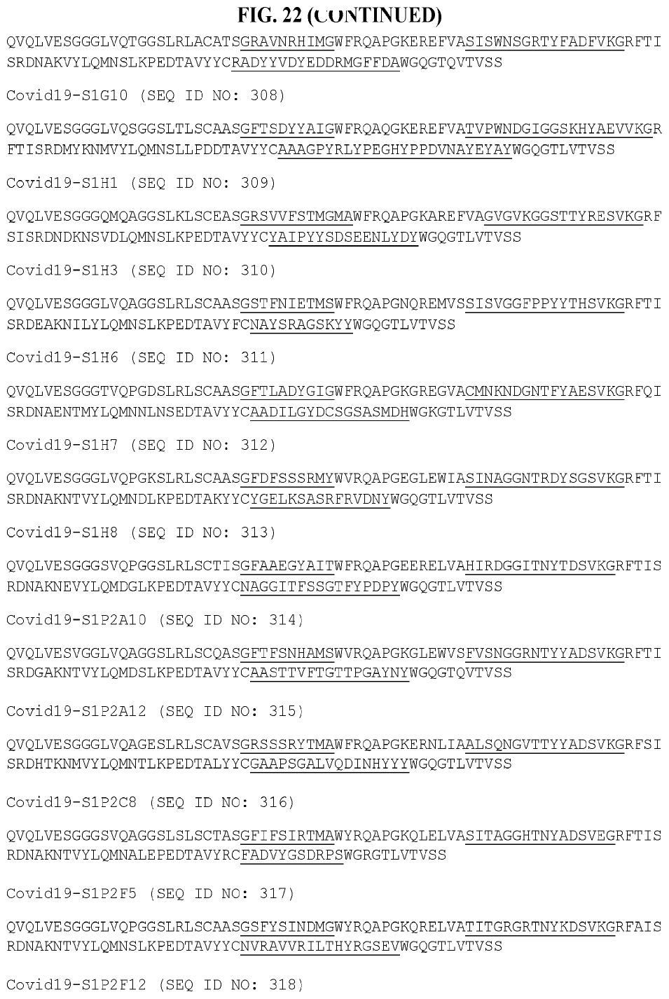

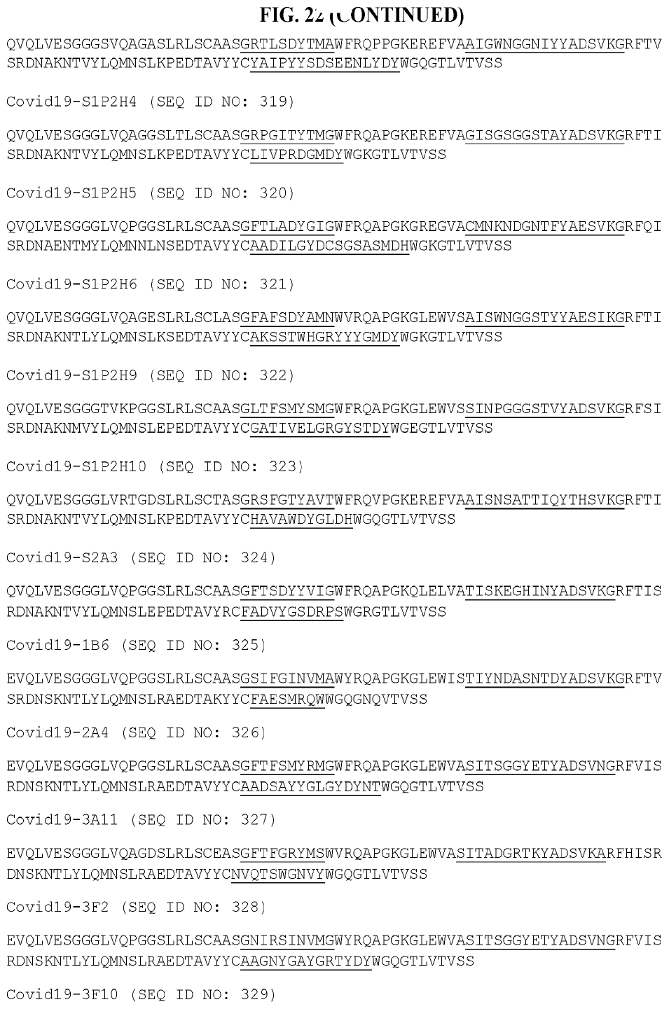

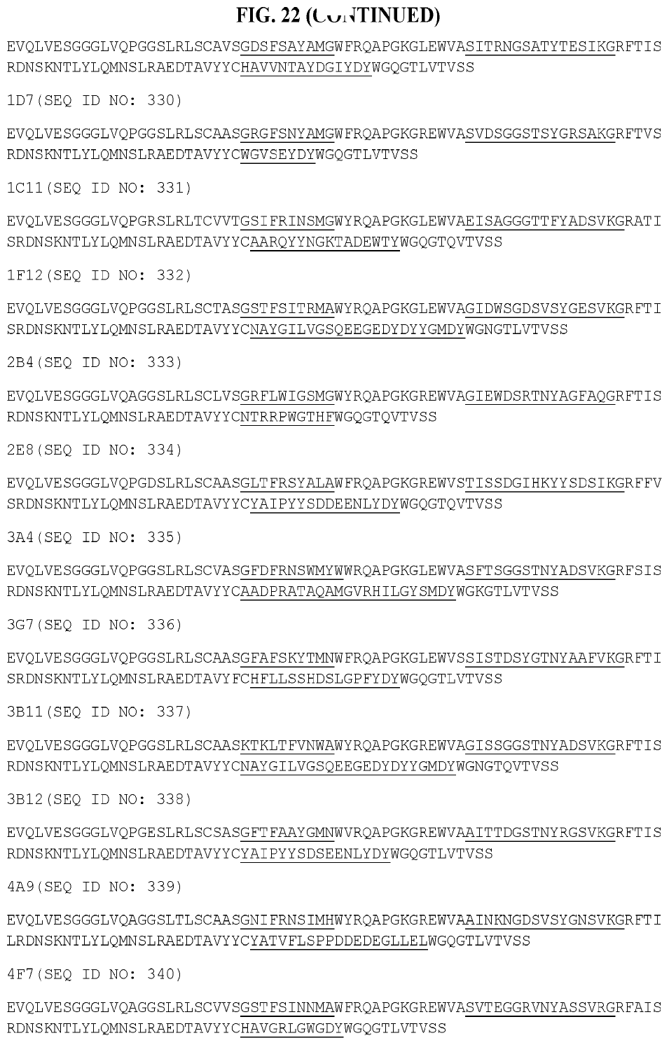

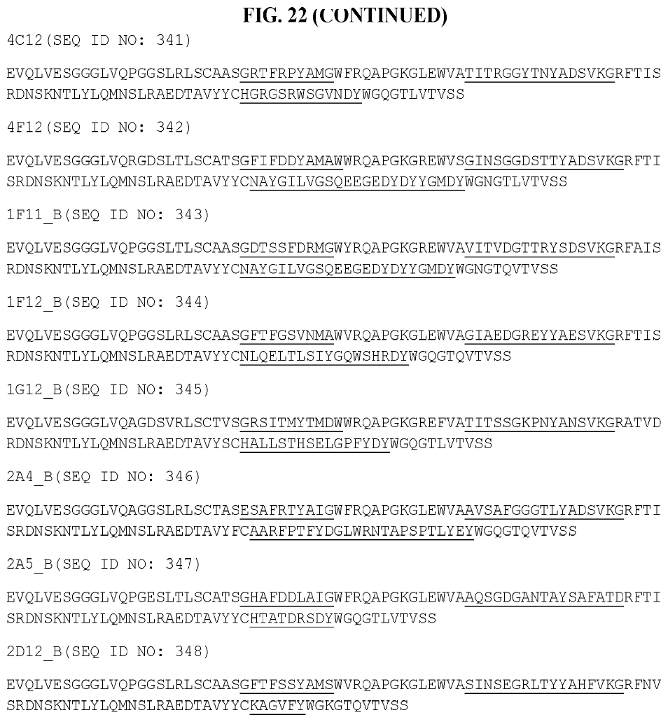

The disclosure also provides nucleic acid comprising a polynucleotide encoding a polypeptide comprising an immunoglobulin heavy chain single variable domain (VHH). The VHH comprises CDRs as shown in FIG. 21, or has sequences as shown in FIG. 22.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

In some embodiments, the antibody or antigen-binding fragment thereof comprises a heavy chain single variable domain (VHH) CDR1 selected from SEQ ID NO: 1, 4, 7, 10, 13, 16, 19, 22, 25, 28, 31, 34, 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142, 145, 148, 151, 154, 157, 160, 163, 166, 169, 172, 175, 178, 181, 184, 187, 190, 193, 196, 199, 202, 205, 208, 211, 214, 217, 220, 223, 226, 229, 232, 235, 238, 241, 244, 247, 250, 253, 256, or 259.

In some embodiments, the antibody or antigen-binding fragment thereof comprises a heavy chain single variable domain (VHH) CDR2 selected from SEQ ID NO: 2, 5, 8, 11, 14, 17, 20, 23, 26, 29, 32, 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140, 143, 146, 149, 152, 155, 158, 161, 164, 167, 170, 173, 176, 179, 182, 185, 188, 191, 194, 197, 200, 203, 206, 209, 212, 215, 218, 221, 224, 227, 230, 233, 236, 239, 242, 245, 248, 251, 254, 257, or 260.

In some embodiments, the antibody or antigen-binding fragment thereof comprises a heavy chain single variable domain (VHH) CDR3 selected from SEQ ID NO: 3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, or 261.

### Antibody Characteristics

The antibodies or antigen-binding fragments thereof described herein can block the binding between the coronavirus (e.g., SARS-CoV-2, SARS-CoV, or MERS-CoV) S protein and ACE2. In some embodiments, by binding to coronavirus S protein, the antibody can neutralize coronavirus. In some embodiments, the antibody can promote virus aggregation. In some embodiments, the antibody can induce Fc-dependent antiviral functions. In some embodiments, the antibody can inhibit cleavage of the S protein by host cell TMPRSS2. In some embodiments, the antibody can block viral entry into host cell.

The disclosure provides antibodies or antigen-binding fragments thereof that block the coronavirus (e.g., SARS-CoV-2, SARS-CoV, or MERS-CoV) S protein and ACE2 (S/ACE) interaction such that the remaining S/ACE binding is less than or about 95%, less than or about 90%, less than or about 85%, less than or about 80%, less than or about 75%, less than or about 70%, less than or about 65%, less than or about 60%, less than or about 55%, less than or about 50%, less than or about 45%, less than or about 40%, less than or about 35%, less than or about 30%, less than or about 25%, less than or about 20%, less than or about 15%, less than or about 10%, less than or about 10%, or less than or about 5% as compared to the S/ACE binding when no antibodies or antigen-binding fragments thereof as described herein are present.

The disclosure provides antibodies or antigen-binding fragments thereof that neutralize the coronavirus (e.g., SARS-CoV-2, SARS-CoV, or MERS-CoV) such that the neutralized coronavirus is at least or about 5%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, or at least or about 95% of the total amount of the coronavirus.

The disclosure provides antibodies or antigen-binding fragments thereof that promote the coronavirus (e.g., SARS-CoV-2, SARS-CoV, or MERS-CoV) aggregation by at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least or about 20 folds, at least or about 30 folds, at least or about 40 folds, at least or about 50 folds, or at least or about 100 folds as compared when no antibodies or antigen-binding fragments thereof as described herein are present.

The disclosure provides antibodies or antigen-binding fragments thereof comprising a human Fc domain, which induce Fc-dependent antiviral functions by at least or about at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least or about 20 folds, at least or about 30 folds, at least or about 40 folds, at least or about 50 folds, or at least or about 100 folds as compared when no antibodies or antigen-binding fragments thereof as described herein are present.

The disclosure provides antibodies or antigen-binding fragments thereof comprising a human Fc domain, which induce host immune response by at least or about at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least or about 20 folds, at least or about 30 folds, at least or about 40 folds, at least or about 50 folds, or at least or about 100 folds as compared when no antibodies or antigen-binding fragments thereof as described herein are present.

The disclosure provides antibodies or antigen-binding fragments thereof that block viral entry (or internalization) into host cell such that the internalization rate is less than or about 50%, less than or about 45%, less than or about 40%, less than or about 35%, less than or about 30%, less than or about 25%, less than or about 20%, less than or about 15%, less than or about 10%, less than or about 10%, or less than or about 5% of the internalization rate when no antibodies or antigen-binding fragments thereof as described herein are present.

In some embodiments, provided herein is an antibody or antigen-binding fragment thereof comprising a single heavy chain. In some embodiments, provided herein is an antibody or antigen-binding fragment thereof comprising a pair of heavy chains. In some embodiments, the heavy chain pair is linked by disulfide bonds. In some embodiments, the heavy chain pair comprises knob-in-hole modifications. In some embodiments, the heavy chain comprises a human IgG Fc domain. In some embodiments, the antibody or antigen-binding fragment thereof comprises in each heavy chain at least 1, 2, 3, 4, 5, 6, 7, 8, 9, or 10 VHH domains. In some embodiments, the VHH domains in each heavy chain specifically bind to the same epitope. In some embodiments, the VHH domains in each heavy chain specifically bind to different epitopes. In some embodiments, the VHH domains in each heavy chain bind to at least 1, 2, 3, 4, or 5 different epitopes. In some embodiments, the epitope is in a receptor-binding domain of coronavirus (e.g., SARS-CoV-2, SARS-CoV, or MERS-CoV) S protein.

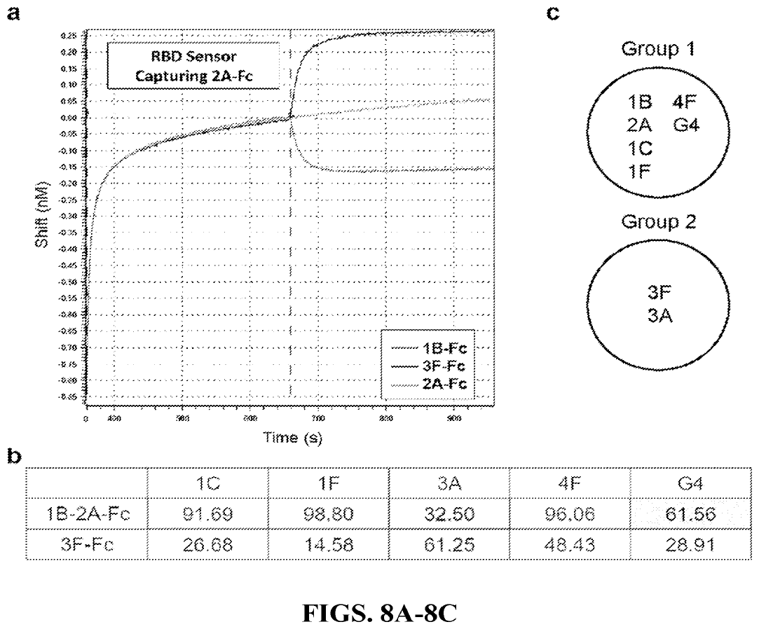

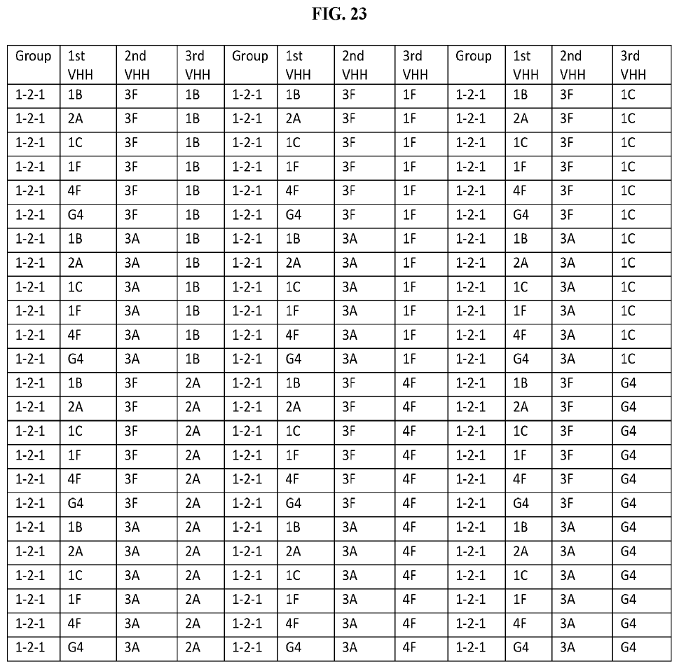

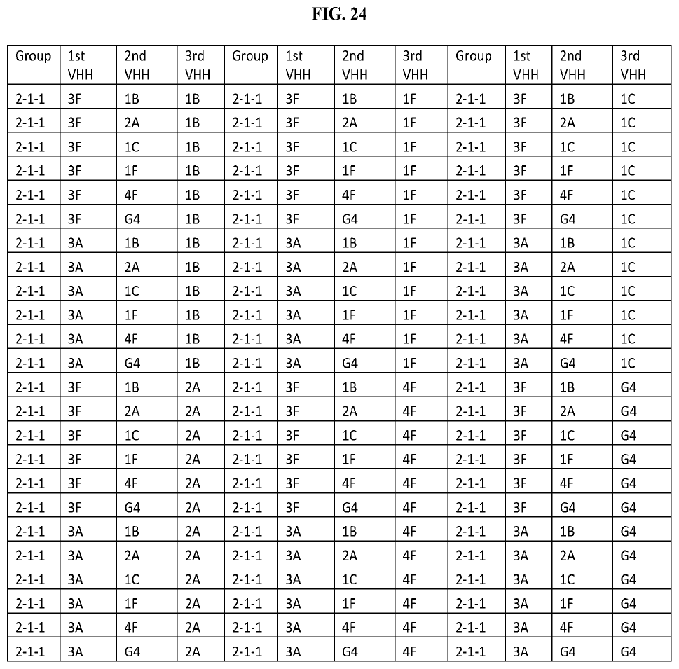

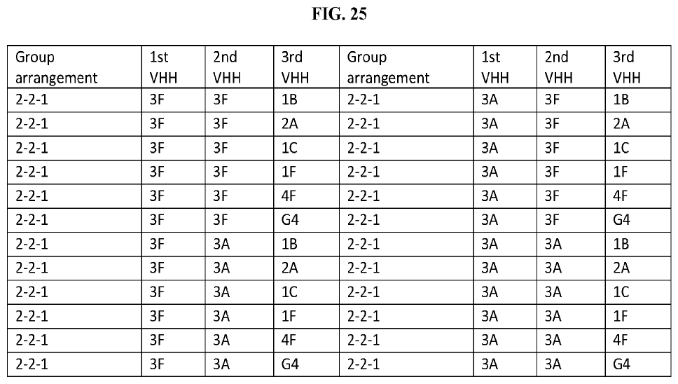

In some embodiments, the antibody or antigen-binding fragment thereof is a tri-specific antibody. In some embodiments, the tri-specific antibody is a tri-specific VHH-Fc. In some embodiments, the tri-specific antibody comprises the same VHHs. In some embodiments, the tri-specific antibody comprises different VHHs. In some embodiments, the VHHs bind to the same epitope (e.g., the same region of SARS-CoV-2 S protein RBD). In some embodiments, the VHHs bind to different epitopes (e.g., different regions of SARS-CoV-2 S protein RBD). In some embodiments, the tri-specific antibody comprises three VHHs, and the three VHHs are selected from VHHs in Group 1 (or G1; e.g., 1B, 2A, 1C, 1F, 4F, and G4) and Group 2 (or G2; e.g., 3F and 3A) as shown in FIG. 8C. In some embodiments, the three VHHs are joined in an order of (e.g., from N-terminus to C-terminus): G1-G1-G1, G1-G1-G2, G1-G2-G1, G1-G2-G2, G2-G1-G1, G2-G1-G2, G2-G2-G1, or G2-G2-G2. In some embodiments, the specific combinations of VHHs (e.g., from N-terminus to C-terminus) is one of the combinations as shown in FIGS. 23-25.

In some embodiments, the antibody or antigen-binding fragment thereof has 4 VHHs. In some embodiments, in order to increase developability of the anti-SARS-CoV-2 multi-specific antibodies, 4 VHHs are combined without the addition of IgG Fc domain to construct tetra-specific VHHs. These molecules would have the added advantage of increased affinity and avidity towards SARS-CoV-2 51 protein compared to bi- and tri-specific VHH-Fcs, despite lacking the Fc effector functions. These tetra-specific antibodies would be ideally suited as antibody prophylactic to prevent the SARS-CoV-2 infection in humans because due to their llama VHH-only structure they would have increased thermostability, easier combination capability, and the possibility of easy large-scale manufacturing using cost-effective expression systems such as Yeast. In some embodiments, the four VHHs are joined in an order of (e.g., from N-terminus to C-terminus): G1-G1-G1-G1, G1-G1-G1-G2, G1-G1-G2-G1, G1-G1-G2-G2, G1-G2-G1-G1, G1-G2-G1-G2, G1-G2-G2-G1, G1-G2-G2-G2, G2-G1-G1-G1, G2-G1-G1-G2, G2-G1-G2-G1, G2-G1-G2-G2, G2-G2-G1-G1, G2-G2-G1-G2, G2-G2-G2-G1, or G2-G2-G2-G2.

In some embodiments, the antibody or antigen-binding fragment thereof is a bi-specific antibody, or tri-specific antibody. In some embodiments, the antibody or antigen-binding fragment thereof can specifically bind to at least 4, 5, or 6 antigens.

In some embodiments, the antibody (or antigen-binding fragment thereof) specifically binds to the S protein (e.g., SARS-CoV-2 S protein, SARS-CoV S protein, or MERS-CoV S protein) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶M, less than 1×10⁻⁷ M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, or 0.05 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

In some embodiments, the antibody (or antigen-binding fragment thereof) specifically binds to the S protein (e.g., SARS-CoV-2 S protein, SARS-CoV S protein, or MERS-CoV S protein) with an IC₅₀ value less than 100 nM, 50 nM, 10 nM, 5 nM, 4 nM, 3 nM, 2 nM, or 1 nM. In some embodiments, the IC₅₀ value is less than 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, or 0.1 μg/ml.

In some embodiments, the tri-specific VHH antibody described herein have a lower aggregation potential (e.g., based on DLS) than a bi-specific VHH antibody comprising at least one identical VHH of the tri-specific VHH antibody. In some embodiments, the tri-specific VHH antibody described herein have a lower aggregation potential (e.g., based on DLS) than a VHH antibody comprising an identical VHH of the tri-specific VHH antibody.

In some embodiments, the tri-specific VHH antibody described herein is more thermostable than a bi-specific VHH antibody comprising at least one identical VHH of the tri-specific VHH antibody. In some embodiments, the tri-specific VHH antibody described herein have is more thermostable than a VHH antibody comprising an identical VHH of the tri-specific VHH antibody.

In some embodiments, the tri-specific VHH antibody (e.g., tri-specific VHH-Fc) binds to coronavirus S protein (e.g., SARS-CoV-2 S1 protein RBD) with a binding affinity that is at least or about 1 fold, 2 folds, 3 folds, 4 folds, 5 folds, 6 folds, 7 folds, 8 folds, 9 folds, 10 folds, 15 folds, 20 folds, 30 folds, 40 folds, 50 folds, 100 folds, or higher than that of a bi-specific or mono-specific VHH antibody comprising at least one identical VHH of the tri-specific VHH antibody. In some embodiments, the tri-specific VHH antibody (e.g., tri-specific VHH-Fc) blocks coronavirus S protein/ACE2 interaction with a blocking potency that is at least or about 1 fold, 2 folds, 3 folds, 4 folds, 5 folds, 6 folds, 7 folds, 8 folds, 9 folds, 10 folds, 15 folds, 20 folds, 30 folds, 40 folds, 50 folds, 100 folds, or higher than that of a bi-specific or mono-specific VHH antibody comprising at least one identical VHH of the tri-specific VHH antibody.

In some embodiments, the coronavirus S protein (e.g., SARS-CoV-2 S1 protein RBD) binding property and/or coronavirus S protein/ACE2 blocking ability of the multi-specific VHH antibody (e.g., tri-specific VHH antibody) is determined by the physical arrangement and/or binding orientation of the multi-specific VHH antibody.

In some embodiments, the antibody or antigen-binding fragment thereof described herein processes more than one mechanisms to neutralize coronavirus infection.

Avidity refers specifically to the strengthening of binding through more than one point of interaction. This effect can be quantified as the ratio of the dissociation constant, for the intrinsic affinity over that for the functional affinity. Sometimes the term avidity is used in a looser sense as a synonym for affinity, in practice particularly for functional affinity, as in the terms avidity assay and avidity index. In some embodiments, the antibody or antigen-binding fragment thereof comprises more than one antigen-binding units (e.g., VHH domains), which confers a better affinity and avidity than an antibody that consists of a single antigen-binding unit.

General techniques for measuring the affinity and/or avidity of an antibody for an antigen include, e.g., ELISA, RIA, and surface plasmon resonance (SPR). In some embodiments, the antibody binds to SARS-CoV-2 S protein, SARS-CoV S protein, MERS-CoV S protein, or other coronavirus S proteins. In some embodiments, the antibody does not bind to other coronavirus S proteins.

In some embodiments, thermal stabilities are determined. The antibodies or antigen-binding fragments as described herein can have a Tm (melting temperature) greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. The antibodies or antigen-binding fragments as described herein can have a Tagg (aggregation temperature, e.g., Tagg at 266 nm (Tagg266) or Tagg at 473 nm (Tagg473)) great than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tagg is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies or antigen binding fragments have a functional Fc region. In some embodiments, effector function of a functional Fc region is phagocytosis.

In some embodiments, the Fc region is human IgG1, human IgG2, human IgG3, or human IgG4.

In some embodiments, the antibodies or antigen binding fragments do not have an Fc region. For example, the antibody (or antigen-binding fragment thereof) is a polypeptide comprising one or more VHH domains that are interconnected by linker peptides. In some embodiments, the antibody comprises at least 1, 2, 3, 4, 5, 6, 7, 8, 9, or 10 VHH domain. In some embodiments, the VHH domains specifically bind to the same epitope. In some embodiments, the VHH domains bind to different epitopes. In some embodiments, the VHH domains bind to at least 1, 2, 3, 4, or 5 different epitopes.

In some embodiments, the antibodies or antigen binding fragments do not have a functional Fc region. In some embodiments, the Fc region has LALA mutations (L234A and L235A mutations in EU numbering), or LALA-PG mutations (L234A, L235A, P329G mutations in EU numbering).

### Methods of Making Anti-Coronavirus S Protein Antibodies

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., SARS-CoV-2 S protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites. VHH domains described herein can be derived from camelids (e.g., llama, and camels) or cartilaginous fishes (e.g., sharks, rays, skates, and sawfish).

Humanized antibodies include antibodies having variable and constant regions derived from (or having the same amino acid sequence as those derived from) human germline immunoglobulin sequences of human immunoglobulin scaffold sequences. Humanized antibodies may include amino acid residues not encoded by human germline immunoglobulin sequences (e.g., mutations introduced by random or site-specific mutagenesis in vitro or by somatic mutation in vivo). Accordingly, “humanized” antibodies are chimeric antibodies wherein sequences from a non-human species are substituted by the corresponding human sequences.

Ordinarily, amino acid sequence variants of the human, humanized, or chimeric anti-coronavirus (e.g., SARS-CoV-2) S protein antibody will contain an amino acid sequence having at least 75%, 80%, 85%, 90%, 95%, 96%, 97%, 98%, or 99% percent identity with a sequence present in the VHH domain of the original antibody.

Identity or homology with respect to an original sequence is usually the percentage of amino acid residues present within the candidate sequence that are identical with a sequence present within the human, humanized, or chimeric anti-coronavirus (e.g., SARS-CoV-2) S protein antibody or fragment, after aligning the sequences and introducing gaps, if necessary, to achieve the maximum percent sequence identity, and not considering any conservative substitutions as part of the sequence identity.

Additional modifications to the anti-coronavirus S protein antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fc regions.

In some embodiments, a covalent modification can be made to the anti-coronavirus (e.g., SARS-CoV-2) S protein antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

In some embodiments, the antibody or antigen-binding fragment thereof is expressed in yeast, insect cells, or mammalian cells (e.g., CHO cells).

In some embodiments, the production, optimization, and testing of the multi-specific VHH antibody (e.g., tri-specific VHH-Fc) described herein is accomplished within less than 6 months, 5 months, 4 months, 3 months, 2 months, or less time.

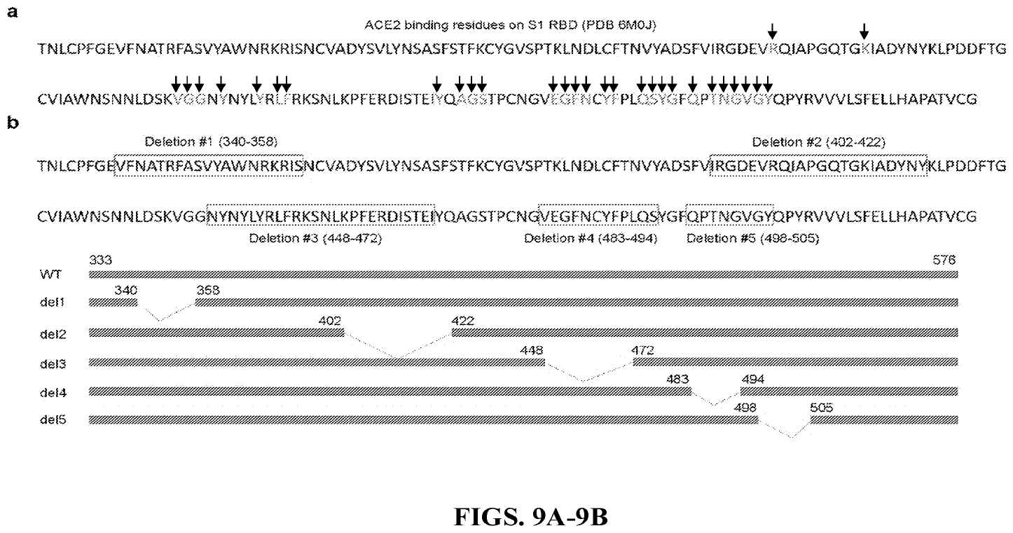

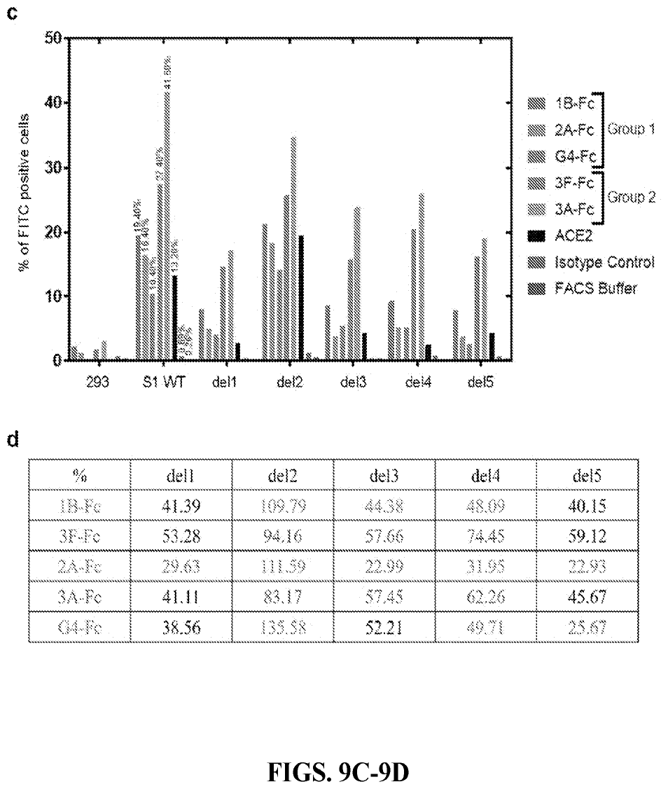

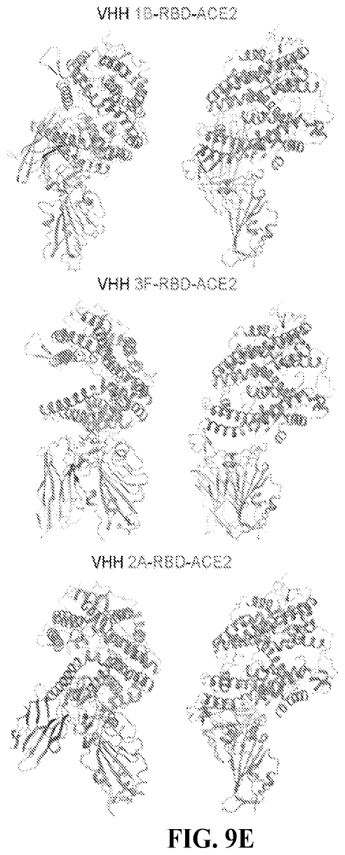

In some embodiments, the binding epitopes for VHH-Fcs (e.g., 1B, 2A, 1C, 1F, 4F, G4, B38, CB6, or P2B-2F6) are within del3, del4, and/or del5 regions of S1 RBD as shown in FIG. 9A. In some embodiments, the binding epitopes for VHH-Fcs (e.g., 1B, 2A, 1C, 1F, 4F, G4, B38, CB6, or P2B-2F6) are within 1, 2, 3, 4, 5, 6, 7, 8, 9, 10, 11, 12, 13, 14, 15, 16, 17, 18, 19, or 20 amino acids of del3, del4, and/or del5 regions of S1 RBD as shown in FIG. 9A.

In some embodiments, the binding epitopes for VHH-Fcs (e.g., 3F, 3A, or CB6) are within the del2 region of S1 RBD as shown in FIG. 9A. In some embodiments, the binding epitopes for VHH-Fcs (e.g., 3F, 3A, or CB6) are within 1, 2, 3, 4, 5, 6, 7, 8, 9, 10, 11, 12, 13, 14, 15, 16, 17, 18, 19, or 20 amino acids of the del2 region of S1 RBD as shown in FIG. 9A.

In some embodiments, the binding epitopes for VHH-Fcs (e.g., S309 or BD-23) are within the del1 region of S1 RBD as shown in FIG. 9A. In some embodiments, the binding epitopes for VHH-Fcs (e.g., S309 or BD-23) are within 1, 2, 3, 4, 5, 6, 7, 8, 9, 10, 11, 12, 13, 14, 15, 16, 17, 18, 19, or 20 amino acids of the del1 region of S1 RBD as shown in FIG. 9A.

### Methods of Treatment and Diagnosis

The antibodies or antigen-binding fragments thereof of the present disclosure can be used for various therapeutic purposes.

In one aspect, the disclosure provides methods for treating a coronavirus-related disease in a subject, methods of neutralizing a coronavirus, methods of blocking a coronavirus/ACE2 interaction, methods of promoting coronavirus aggregation, methods of inducing Fc-dependent antiviral functions, methods of blocking internalization of a coronavirus by a cell, methods of identifying a subject having a coronavirus-related disease. In some embodiments, the treatment can halt, slow, retard, or inhibit progression of a coronavirus-related disease. In some embodiments, the treatment can result in the reduction of in the number, severity, and/or duration of one or more symptoms of the coronavirus-related disease in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a coronavirus-related disease).

In some embodiments, the coronavirus-related disease is COVID-19 (Coronavirus disease 2019), Severe acute respiratory syndrome (SARS), or Middle East respiratory syndrome (MERS).

In some embodiments, the coronavirus that causing the coronavirus-related disease is SARS-CoV, SARS-CoV-2, MERS-CoV, or other types of coronavirus having one or more S proteins. In some embodiments, the amino acid sequence of the S protein of the coronavirus described herein comprises a sequence that is at least or about 50%, at least or about 55%, at least or about 60%, at least or about 65%, at least or about 70%, at least or about 75%, at least or about 80%, at least or about 85%, at least or about 90%, at least or about 95%, or at least or about 98% identical to the receptor-biding domain sequence of the SARS-CoV-2 S protein.

In some embodiments, the compositions and methods disclosed herein can be used for treatment of patients at risk for a coronavirus-related disease. Patients with coronavirus-related disease can be identified with various methods known in the art.

As used herein, by an “effective amount” is meant an amount or dosage sufficient to effect beneficial or desired results including halting, slowing, retarding, or inhibiting progression of a disease, e.g., a coronavirus-related disease. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of a coronavirus-related disease in a patient. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal.

A typical daily dosage of an effective amount of an antibody is 0.01 mg/kg to 100 mg/kg (mg per kg of patient weight). In some embodiments, the dosage can be less than 100 mg/kg, 50 mg/kg, 25 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 25 mg/kg, 20 mg/kg, 15 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 25 mg/kg, 20 mg/kg, 15 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments, the multi-specific VHH antibody (e.g., tri-specific VHH-Fcs) is administered at the dosage described herein without affecting body weight and/or survival of the subject.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of the coronavirus-related disease). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, the antibodies or antigen-binding fragments thereof can be used for detecting coronavirus (e.g., SARS-CoV-2, SARS-CoV, or MERS-CoV) in a subject (e.g., a human) or diagnosing a coronavirus related disease. Methods known in the art can be designed, e.g., ELISA, to produce a diagnostic test kit. In some embodiments, one or more antibodies or antigen-binding fragments comprising any of the heavy chain single variable domains as described herein can be used.

In some embodiments, the multi-specific VHH antibody (e.g., tri-specific VHH-Fcs) are administered to a subject (e.g., a human) at a lower concentration than a bi-specific or mono-specific VHH antibody comprising at least one identical VHH domain with the multi-specific VHH antibody.

In some embodiments, the multi-specific VHH antibody (e.g., tri-specific VHH-Fcs) binds to at least one (e.g., 1, 2, 3, 4, 5, 6, 7, 8, 9, or 10) coronavirus S protein (e.g., SARS-CoV-2 S protein) from the same virus. In some embodiments, the multi-specific VHH antibody (e.g., tri-specific VHH-Fcs) binds to at least one (e.g., 1, 2, 3, 4, 5, 6, 7, 8, 9, or 10) coronavirus S protein (e.g., SARS-CoV-2 S protein) from different viruses. In some embodiments, a single multi-specific VHH antibody (e.g., tri-specific VHH-Fcs) binds to 1, 2, 3, 4, 5, 6, 7, 8, 9, or 10 coronaviruses. In some embodiments, the multi-specific VHH antibody (e.g., tri-specific VHH-Fcs) binds to at least one mutated coronavirus S protein (e.g., mutated SARS-CoV-2 S protein). In some embodiments, the multi-specific VHH antibody (e.g., tri-specific VHH-Fcs) binds to at least one mutated and at least one wild-type coronavirus S protein (e.g., SARS-CoV-2 S protein).

In some embodiments, the multi-specific VHH antibody (e.g., tri-specific VHH-Fcs can be delivered to a subject by intranasal administration or intraperitoneal administration. In some embodiments, administration of the multi-specific VHH antibody decreases the viral titer (e.g., in lungs) in the subject to less than 95%, less than 90%, less than 80%, less than 70%, less than 60%, less than 50%, less than 40%, less than 30%, less than 20%, less than 10%, less than 5%, or less than 1% as compared to the viral titer of a subject without the administration. In some embodiments, the viral titer is about 10¹¹, about 10¹⁰, about 10⁹, about 10⁸, about 10⁷, about 10⁶, about 10⁵, about 10⁴, about 10³, about 10², or about 10 copies per gram of the collected sample (e.g., lungs). In some embodiments, the viral titer is determined 1 day, 2 days, 3 days, 4 days, or 5 days post virus infection.

In some embodiments, the S protein has one or more mutations, e.g., in the RBD. In some embodiments, the one or more mutations are located at K417, E484, N501, D614, and/or N501 of the wild-type S protein. In some embodiments, the one or more mutations are K417N, E484K, N501Y, D614G, and/or N501Y. In some embodiments, the multi-specific VHH antibody described herein (e.g., tri-specific VHH-Fcs) has similar binding and ACE2 blocking functions against an S protein having one or more mutations (e.g., K417N, E484K, N501Y, D614G, and/or N501Y). In some embodiments, the KD value of the multi-specific VHH antibody described herein (e.g., tri-specific VHH-Fcs) binding to an S protein having one or more mutations (e.g., K417N, E484K, N501Y, D614G, and/or N501Y) is great than 10%, greater than 20%, greater than 30%, greater than 40%, greater than 50%, greater than 60%, greater than 70%, or greater than 80% of the KD value binding to a wild-type S protein.

### Administration Through Respiratory Tract

The compositions as described herein can be administered through respiratory tract by various means, for example, nasal administration, nasal instillation, insufflation (e.g., nasal sprays), inhalation (through nose or mouth), intrapulmonary administration, intratracheal administration, or any combinations thereof. As used herein, the term “nasal instillation” refers to a procedure that delivers a therapeutic agent directly into the nose and onto the nasal membranes, wherein a portion of the therapeutic agent can pass through tracheas and is delivered into the lung.

Because of the occasionally limited functionally for the lungs that are in need of treatment, a therapeutic agent sometimes cannot be effectively delivered to the target sites in the lungs (e.g., bronchioles or alveoli) through respiratory tract administration. In these cases, an agent that can clear the airways can be administered to the subject first. In some embodiments, these agents can induce dilation of bronchial passages, and/or vasodilation in muscle. Such agents include, but are not limited to, beta2 adrenergic receptor agonists, anticholinergic agents, corticosteroids. In some embodiments, an agent for treating asthma can be used.

Pharmaceutical compositions suitable for administering through respiratory tract can include, e.g., liquid solutions, aqueous solutions (where water soluble), or dispersions, etc. In some embodiments, these compositions can comprise one or more surfactants.

As used herein, the term “respiratory tract” refers to the air passages from the nose to the pulmonary alveoli, including the nose, throat, pharynx, larynx, trachea, bronchi, and any part of the lungs. In some embodiments, the composition is administered to the lungs or any part of the respiratory system.

In some embodiments, the compositions can be administered a subject by a delivery system that can convert the composition into an aerosol form, e.g., a nebulizer, a vaporizer, a nasal sprayer, an inhaler, a soft mist inhaler, a jet nebulizer, an ultrasonic wave nebulizer, a pressurized metered dose inhaler, a breath activated pressurized metered dose inhaler, or a vibrating mesh device. As used herein, the term “inhaler” refers to a device for administering compositions in the form of a spray or dry powder that is inhaled (breathed in either naturally or mechanically forced in to the lungs) through the nose or mouth. In some embodiments, inhalers include e.g., a passive or active ventilator (mechanical with or without an endotracheal tube), nebulizer, dry powder inhaler, metered dose inhaler, and pressurized metered dose inhaler. Once the antibodies (or antigen-binding fragments thereof) are deposited or localized near cells, a subset of the antibodies can neutralize the coronavirus as described herein.

In some embodiments, the devices can use air (e.g., oxygen, compressed air) or ultrasonic power to break up solutions and suspensions into small aerosol particles (e.g., droplets) that can be directly inhaled from the mouthpiece of the device. In some embodiments, the devices use a mesh/membrane with laser drilled holes (e.g., from 1000 to 7000 holes) that vibrates at the top of the liquid reservoir, and thereby pressures out a mist of very fine droplets through the holes.

The delivery system can also have a unit dose delivery system. The volume of solution or suspension delivered per dose can be anywhere from about 5 to about 2000 microliters, from about 10 to about 1000 microliters, or from about 50 to about 500 microliters. Delivery systems for these various dosage forms can be dropper bottles, plastic squeeze units, atomizers, nebulizers or pharmaceutical aerosols in either unit dose or multiple dose packages.

In some embodiments, the device is a small, hard bottle to which a metered dose sprayer is attached. The metered dose can be delivered by drawing the composition into a chamber of defined volume, which chamber has an aperture dimensioned to aerosolize and aerosol formulation by forming a spray when a liquid in the chamber is compressed. The chamber is compressed to administer the composition. In certain devices, the chamber is a piston arrangement. Such devices are commercially available.

Alternatively, a squeeze bottle with an aperture or opening dimensioned to aerosolize an aerosol formulation by forming a spray when squeezed can be used. The opening is usually found in the top of the bottle, and the top is generally tapered to partially fit in the nasal passages for efficient administration of the aerosol formulation. Preferably, the nasal inhaler can provide a metered amount of the aerosol formulation, for administration of a measured dose of the therapeutic agent.

In some embodiments, the aerosolization of a liquid formulation for inhalation into the lung involves a propellant. The propellant may be any propellant generally used in the art. Specific non-limiting examples of such useful propellants are a chlorofluorocarbon, a hydrofluorocarbon, a hydrochlorofluorocarbon, or a hydrocarbon, including trifluoromethane, dichlorodifluoromethane, dichlorotetrafluoroethanol, and 1,1,1,2-tetrafluoroethane, or combinations thereof.

Pharmaceutically acceptable diluents in such aerosol formulations include but are not limited to sterile water, saline, buffered saline, dextrose solution, and the like. In certain embodiments, a diluent that may be used in the present invention or the pharmaceutical formulation is phosphate buffered saline or a buffered saline solution generally between the pH 7.0-8.0 range (e.g., pH 7.4), or water.

The aerosol formulation also may optionally include pharmaceutically acceptable carriers, diluents, solubilizing or emulsifying agents, surfactants and excipients.

The present disclosure further contemplates aerosol formulations comprising the composition as described herein and another therapeutically effective agent.

The total amount of the composition delivered to the subject will depend upon several factors, including the total amount aerosolized, the type of nebulizer, the particle size, subject breathing patterns, severity of lung disease, and concentration in the aerosolized solution, and length of inhalation therapy. The amount of composition measured in the alveoli may be substantially less than what would be expected to be from the amount of composition present in the aerosol, since a large portion of the composition may be exhaled by the subject or trapped on the interior surfaces of the nebulizer apparatus.

Skilled practitioners will be able to readily design effective protocols, particularly if the particle size of the aerosol is optimized. In some instances, it is useful to administer higher doses when treating more severe conditions. If necessary, the treatment can be repeated on an ad hoc basis depending upon the results achieved. If the treatment is repeated, the mammalian host can be monitored to ensure that there is no adverse immune response to the treatment. The frequency of treatments depends upon a number of factors, such as the amount of composition administered per dose, as well as the health and history of the subject.

### Pharmaceutical Compositions

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., inhibits coronavirus) in a subject (e.g., a human subject identified as having COVID-19), or a subject identified as being at risk of developing the disease (e.g., a subject who has previously infected by coronavirus but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Materials and Methods

### Cell Lines and Transfections

The cell lines used in this study were cultured in media as stated below. Expi293 (Thermo Fisher Scientific)—Expi293 expression medium (Thermo Fisher Scientific), NK-92-CD16 (Natural killer cell line expressing CD16) cells (ATCC)—RPMI 1640, 10% fetal calf serum (FCS), 40 ng/ml IL-2. The cells were maintained in a humidified chamber at 37° C., 8% CO₂. The Expi293 cells were transiently transfected with plasmids expressing SARS-CoV-2 51 protein using the ExpiFectamine 293 transfection reagent (Thermo Fisher Scientific) according to manufacturer's instructions. Briefly, the cells were plated at 1.7×10⁶ cells/ml density overnight in 30 ml of fresh media in 125 ml shake flasks. The following day, 30 μg of DNA and 80 μl of ExpiFectamine 293 were separately mixed in 1.5 ml of Opti-MEM, and incubated at room temperature for 3 minutes. Then, the ExpiFectamine 293 and DNA mixtures were mixed and incubated for another 20 minutes at room temperature. Finally, it was added to flasks containing the cells and incubated in a humidified chamber at 37° C., 8% CO₂. The cells were used for experiments after overnight incubation or frozen in liquid N₂.

### VHH-Fc Expression and Purification

The Expi293 cells were transiently transfected with plasmids expressing VHH-Fcs as stated previously according to manufacturer's instructions. Enhancers were added to cells 17 hours after transfection and they were centrifuged at 3000 g for 10 minutes after 72 hours of transfection. Then, the supernatant was filtered with a 0.45 μm membrane and the antibody concentration was determined using Protein A probe on Gator (Probe Life). Then, the VHH-Fcs were purified using Protein A columns on the AKTA Explorer 100 purification system (buffer A: PBS, pH=7.4; buffer B: 0.1 M Glycine, pH=2.5), and dialyzed in PBS twice. The antibodies were then filtered again with a 0.22 μM membrane and used for experiments.

### Epitope Binning (Competition) Assays

The initial assay was performed using Gator system (Probe Life). After pre-wetting the SARS-CoV-2 S1 RBD sensors in Q Buffer (Probe Life), the sensor captured 30 μg/ml of the first monoclonal VHH-Fc 2A for about 300 seconds, then the loaded sensor captured 10 μg/ml of the second monoclonal VHH-Fc, either 1B, 3F, or 2A, which was quantified over time by Gator.

The follow-up assay for VHH-Fcs 1B-2A and 3F was performed by ELISA. A 96-well plate was coated to a final concentration of 1 μg/ml of SARS-CoV-2 S1 protein and placed overnight at 4° C. Then, 60 μg/ml 1B-2A-Fc or 3F-Fc were premixed with each competing c-Myc tagged VHHs from periplasmic supernatant at a 1:1 ratio. After another one hour of incubation, a biotinylated anti-c-Myc antibody (9E10) was added and the samples were incubated for another one hour. Then, streptavidin-HRP was added followed by the treatment with Amplex Red (Thermo Fisher Scientific) and 30% H₂O₂ containing development solution. The emitted signal for each sample was detected by using a fluorescence plate reader (SpectraMax Gemini XPS). The percent difference from the competing pairs versus the VHH-Fc alone signal was calculated using the following formula; % difference from VHH-Fc signal=(1−((signal of competing pair−no antibody signal)/(signal of VHH-Fc alone−no antibody signal))*100.

### In-Vitro S1 Protein Binding Assay

The 96-well ELISA plates (Greiner Bio-One) were directly coated with SARS-CoV-2 S1 protein (Acro Biosystems) diluted in PBS at 1 μg/ml and incubated overnight at 4° C. Then, the plates were washed with PBS containing 0.5% Tween20 (PBST) and blocked with 1% bovine serum albumin (BSA) in PBS at room temperature for one hour. The plates were washed again with PBST and incubated with the test antibodies at room temperature for one hour. The antibodies were used at 1:5 serial dilutions. The plates were washed with PBST followed by the addition of anti-human-Fc antibodies conjugated to horseradish peroxidase (HRP) (Jackson ImmunoResearch) at 1:5000 dilution in PBST and the plates were incubated at room temperature for 1 hour. Following washing again by PBST, the plates were treated with ELISA development buffer solution containing Amplex Red and 30% H₂O₂. The emitted binding signal for each sample was detected by using a fluorescence plate reader. The blocking was measured in relative fluorescence units (RFU) and the % inhibition was calculated as follows; % Inhibition=(1−(mean experimental value/mean of no antibody control))*100.

### S/ACE2 Blocking Assay

The 96-well ELISA plates (Greiner Bio-One) were coated with SARS-CoV-2 S1 protein (Acro Biosystems) and incubated overnight as stated previously. Then, the plates were washed with PBST and blocked with 2% BSA in PBS at room temperature for one hour. The plates were washed again with PBST and incubated with the test antibodies at room temperature for 45 minutes. The antibodies were used at 1:5 serial dilutions. Then, recombinant biotinylated-ACE2 (Acro Biosystems) was directly added to the plates at 4.65 μg/ul and incubated at room temperature for another 45 minutes. The plates were washed with PBST followed by the addition of streptavidin conjugated to HRP at 1:1000 dilution in PBST. The plates were incubated at room temperature for another 45 minutes. Then, they were washed with PBST and treated with ELISA development buffer. The emitted binding signal for each sample was detected by using a fluorescence plate reader.

### Analysis of Physical Characteristics of VHH-Fcs

The purified VHH-Fcs were analyzed by the UNcle system (Unchained Labs) for their thermostability using differential scanning fluorimetry (DSF) and static light scattering (SLS), and aggregation potential using dynamic light scattering (DLS) assays. The DLS was measured at 25° C. and the data was analyzed using UNcle Analysis Software. For DSF/DLS assays, a temperature ramp of 1° C./min was performed with monitoring from 25° C. to 95° C. SLS was measured by UNcle at 266 nm and 473 nm. Tm and Tagg were analyzed and calculated by the UNcle Analysis Software.

### Pseudovirus Neutralization Assay

Pseudovirus neutralization assay was performed in collaboration with GenScript Biotech (Piscataway, N.J.). Briefly, pseudovirus expressing luciferase and containing SARS-CoV-2 S1 as the envelope glycoprotein in a lentiviral vector was produced in HEK293T cells, and the virus titration was determined by ELISA. HEK293 cells expressing ACE2 receptor and transmembrane Serine protease 2 (TMPRSS2) were used as the target cells, and were seeded in 96-well plates. Then, pseudovirus with the serial dilutions of the antibodies were mixed with the target cells. The cells were incubated for 48 hours at 37° C. and an amount of 30 μl of the cell suspension was transferred to an assay plate. It was mixed with luciferase detection reagents from Bio-Glo™ Luciferase Assay System (Promega) and incubated for 5-10 minutes at room temperature. Then, the luminescence was measured by a plate reader. The background RLU was subtracted from the RLU of the experimental samples. The values for % inhibition were derived from RLU as follows; % Inhibition=(1−(mean of experimental value−mean of cells treated only with buffer)/(mean of cells treated only with SARS-CoV-2))*100.

### Antibody-Dependent Cellular Cytotoxicity (ADCC) Assay

Target Expi293 cells expressing S1 protein (293 SProt) were washed with RPMI media containing 10% Horse serum and 40 ng/ml IL-2, and plated in 96-well plates at 1×10⁴ cells/well density. Then, they were mixed with antibodies at 40 μg/ml of final concentration. Then, NK-92-CD16 cells expressing GFP were added to wells at 3×10⁴ cells/well density (Effector: Target—3:1) and the plates were incubated overnight 37° C., 8% CO₂. Then, the cells were washed twice and resuspended in DPBS with 2% FBS. They were assessed by flow cytometry using a FACSCalibur cytometer (BD Biosciences). 293 SProt and GFP-NK-92-CD16 cells were each used as a reference to set up overall target cell gating and to establish the GFP positive NK-92-CD16 populations, allowing differentiation between the NK-92-CD16 effector cells and 293 SProt target cells. The GFP negative 293 SProt cell percentage was evaluated for all samples. Then, cell death percentage was calculated as follows; % Cell death=(1−(antibody treated cell percentage/average of isotype control percentage))*100.

### Statistical Analysis

The four-parameter non-linear regression analysis from Prism software version 8.4.3 was used for all binding and blocking curves, which also included the IC₅₀ values for the blocking assays. All error bars represented in the data are based on standard deviation, unless otherwise specified.

### Example 1. Discovery of Anti-SARS-CoV-2 Antibody

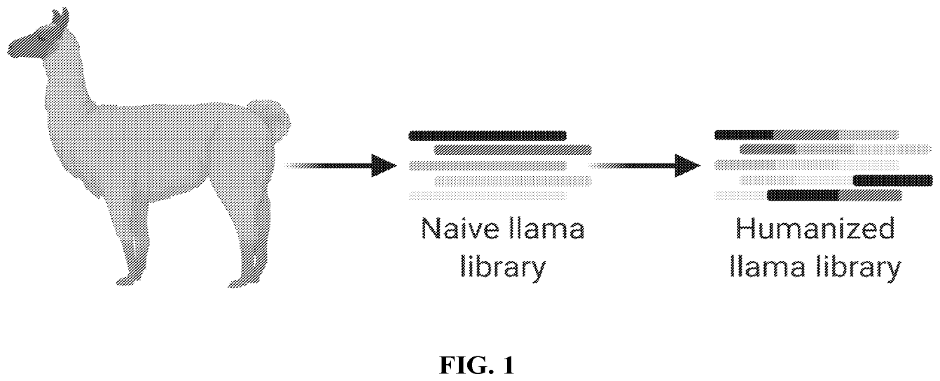

One naïve and one designed synthetic llama VHH libraries were used for this approach, as shown in FIG. 1. PBMC from 65 llamas were obtained, and RNA was isolated to generate cDNA by reverse transcription. Then, the VHH genes were amplified by two rounds of PCR and cloned to a phage display vector to construct the naïve VHH library. The synthetic (e.g., humanized) VHH library was prepared by incorporation of shuffled VHH CDR1, 2 and 3, generated by overlapping PCR, to a modified human VH scaffold to generate enhanced diversity and keep low immunogenicity.

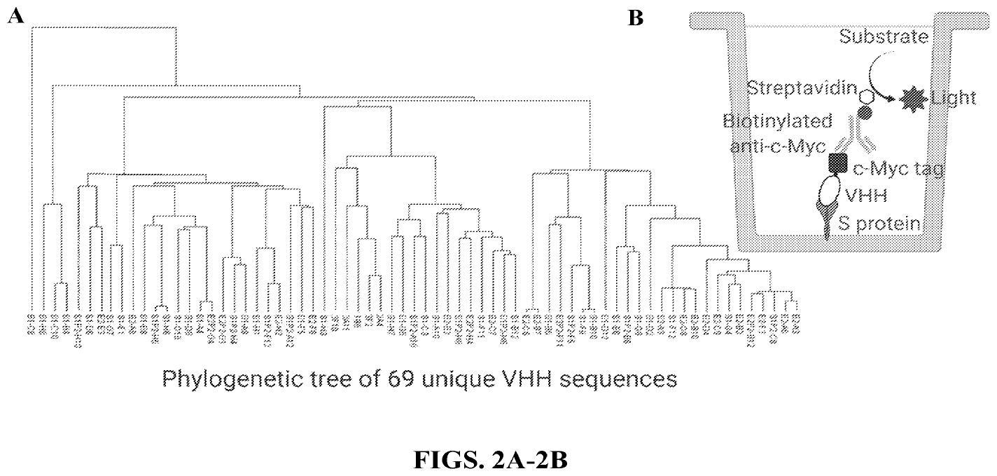

The two llama VHH libraries were panned against recombinant SARS-CoV-2 S protein. Specifically, the VHH phage libraries were used for panning SARS-CoV-2 S1 fused to mouse Fc protein as target antigen. Wells were coated with anti-mouse Fc to immobilize the antigen, and 3 rounds of phage panning were performed with reduced antigen concentration in each round. After panning, 91 high affinity VHH hits for SARS-CoV-2 S protein binding were obtained, among which, 69 were unique sequences (FIG. 2A). The ability of VHHs to bind to recombinant SARS-CoV-2 S proteins were assessed by ELISA. As shown in FIG. 2B, plates were coated with SARS-CoV-2 S1, the bound VHHs were detected by biotinylated anti-c-Myc antibodies and subsequent addition of streptavidin-HRP. Chemiluminescence signal was induced by HRP by adding substrate.

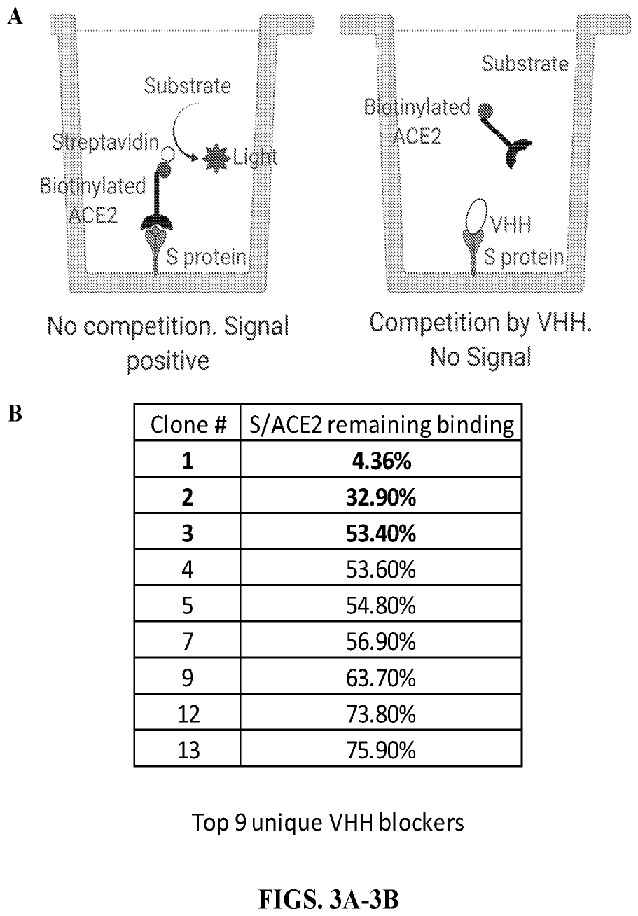

The ability of VHHs to block the SARS-CoV-2 S protein and ACE2 (S/ACE2) interaction in vitro were also assessed, by an ACE2 competition assay. As shown in FIG. 3A, ELISA for ACE2 competition assay was performed by coating the plates with SARS-CoV-2 S1 as described in FIG. 2B, and adding VHH in the presence of biotinylated ACE2. S1/ACE2 blocking function was determined by the reduction of HRP-induced chemiluminescence signal. Results show that 9 out of 69 unique S protein binders exhibited S/ACE2 blocking function, as listed in FIG. 3B.

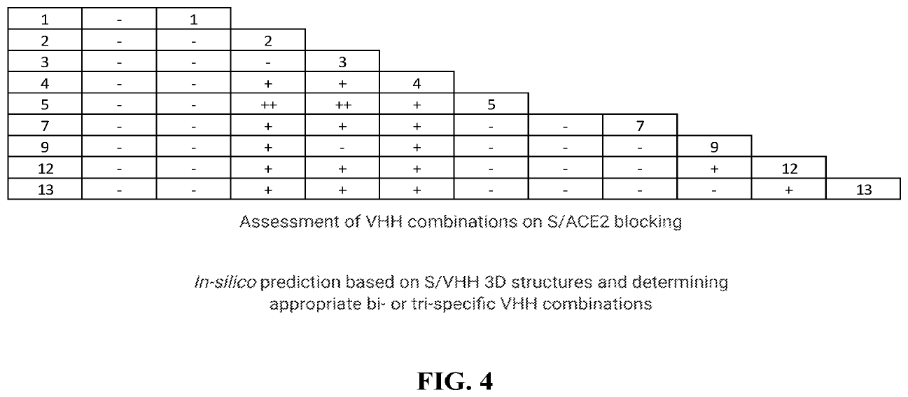

Using the same assays, follow-up studies revealed that pairwise combination of some of the 9 VHH blockers (e.g., clone numbers 2+5; or clone numbers 3+5) led to synergistic blocking efficacy of S/ACE2 interaction. The results are shown in FIG. 4.

### Example 2. Design of Multi-Specific Nanobody-Fc for SARS-CoV-2 Treatment

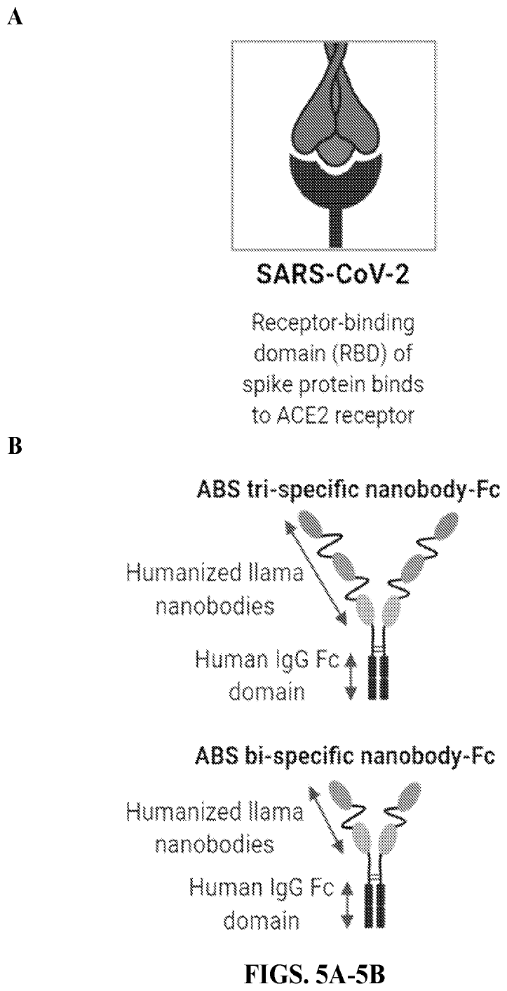

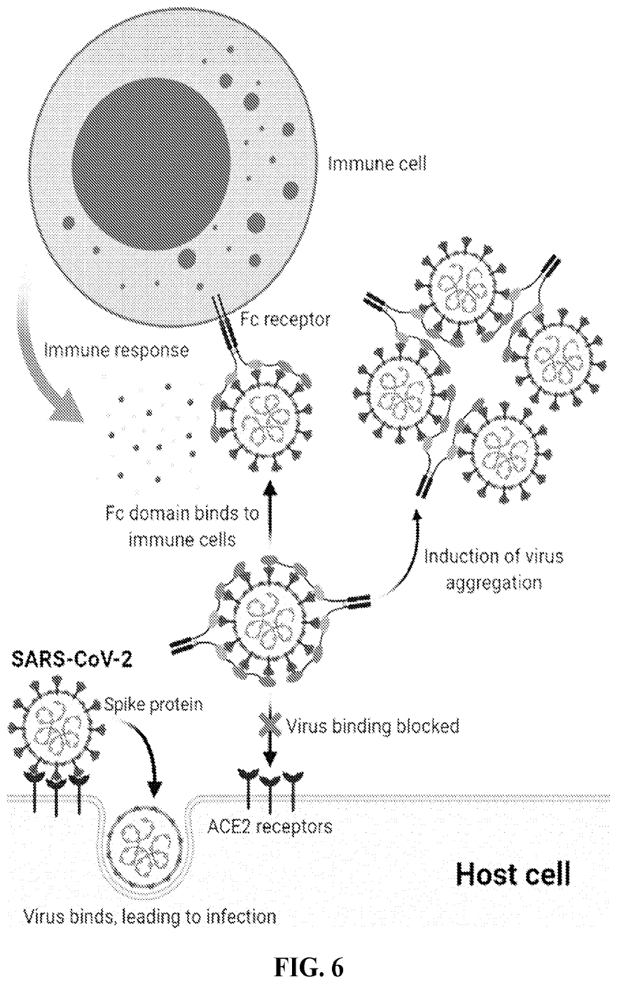

Overall S protein binding affinity and avidity can be improved by selecting and fusing two or three different humanized VHH sequences that target different but adjacent S protein RBD epitopes (FIG. 5A) with high affinity and avidity into a single multi-specific antibody. Such therapeutic antibodies can also present increased S/ACE2 blocking function. As shown in FIG. 5B, this design can be aided by analyzing different VHH combinations in silico with modeled structures via a computer-assisted antibody design (CAAD) approach. CAAD can also be used to further improve the VHH sequences so that the VHHs have low immunogenicity in humans, and high developability and manufacturability. The designed candidates can be analyzed in vitro by binding and blocking assays to determine their S protein binding and S/ACE2 blocking capabilities, respectively. An Fc fusion version of this multi-specific VHH antibody can be generated and its SARS-CoV-2 neutralizing capability can be explored. As shown in FIG. 6, these molecules can neutralize SARS-CoV-2 by multiple mechanisms of action, e.g., blocking S/ACE2 interaction and subsequent virus internalization, promoting virus aggregation, and/or inducing Fc-dependent antiviral functions.

### Example 3. Diagnostic Applications of Anti-SARS-CoV-2 Antibody

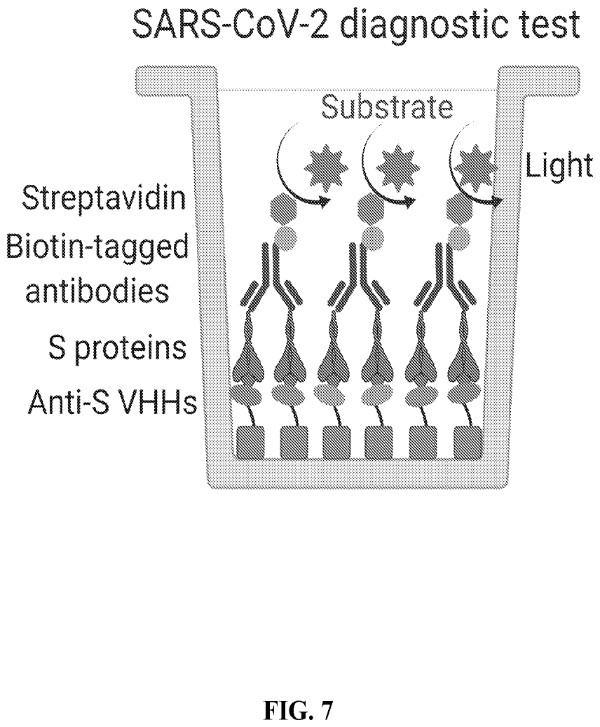

All 69 unique S protein binders can be assessed to probe whether they could be used for diagnostic applications to detect serum S protein and/or SARS-CoV-2 virions, either as single VHHs or in combination (FIG. 7).

### Example 4. Identification of VHHs Binding to Different Epitopes of SARS-CoV-2 S1 Protein RBD

It is hypothesized that the synergistic effect shown in FIG. 4 was caused by binding of the VHHs to different epitopes within the S1 RBD. To test this, epitope binning assays by biolayer interferometry (FIG. 8A) or ELISA (FIG. 8B) on a selected number of candidates were performed.

First, in the epitope binning assay (FIG. 8A), an S1 RBD sensor was used to capture 2A-Fc, followed by incubation with the lead candidates 1B-Fc, 3F-Fc or 2A-Fc, which were fused to human IgG1 Fc domains to render the Fc effector functions against SARS-CoV-2. This analysis showed that 3F-Fc drastically increased the signal compared to the 2A-Fc control, while 1B-Fc drastically decreased the signal compared to the control. This indicates that 3F-Fc does not compete with the 2A-Fc and it is likely that they bind to different S1 RBD epitopes. In contrast, 1B-Fc competed with 2A-Fc, indicating that they compete for binding to the same S1 RBD epitope (FIG. 8A). These results confirm the hypothesis and show that S/ACE2 blocking VHHs bind to at least two separate unique epitopes within the S1 RBD.

Next, an ELISA-based epitope binning assay was performed to assess five additional VHHs (1C, 1F, 3A, 4F, and G4) unfused to Fc, but previously assessed to block the SARS-CoV-2 S/ACE2 interaction. The assessment of more VHHs would categorize several other VHHs into the binding groups, which aids in multi-specific antibody design and construction. In this ELISA, wells were coated with SARS-CoV-2 S1 and incubated with bi-specific VHH-Fc 1B-2A (based on previous data, 1B and 2A likely bind the same epitope) or monoclonal VHH-Fc 3F-Fc (based on previous data, this binds a different epitope than 1B or 2A), premixed with the VHH candidates. The resulting relative fluorescence signals (relative to the VHH-Fc alone signal) obtained for each sample were calculated to reflect the percent difference from 1C, 1F, 3A, 4F, or G4 signals from when the VHHs were combined with 1B-2A-Fc or 3F-Fc (FIG. 8B). The results showed that VHH-Fcs 1C, 1F, and 4F had almost 100% difference from 1B-2A-Fc, suggesting that they compete for the same epitope. However, G4 may partially compete with 1B-2A-Fc, whereas 3A is not likely to compete for the same epitope. Additionally, these results showed that 3A may partially compete with 3F (FIG. 8B). Taken together, the 8 VHH blockers of S/ACE2 interaction can be categorized into 2 major groups based on their epitope binding; Group 1 consists of 6 VHHs, whereas Group 2 consists of 2 VHHs (FIG. 8C).

In the examples, 3F, 1B, 2A, G4, 4F, 1F, 1C, and 3A corresponds to Covid19-3F2, Covid19-1B6, Covid19-E2A6, Covid19-S1G4, 4F12, 1F12, 1C11, and 3A4, respectively in FIG. 22.

### Example 5. Elucidation of Epitopes on S1 RBD that Bind to VHH-Fcs

To elucidate the structural basis of the newly discovered epitope binding groups, structural models for 1B, 3F, and 2A VHHs were computationally generated and docked to an SARS-CoV-2 S1 RBD structure exported from PDB 6M0J using Schrodinger BioLuminate® software. FIG. 9A shows the ACE2 binding residues (under arrow symbols) of the SARS-CoV-2 S1 protein. This approach generated an array of poses of each S1 RBD/VHH complex structure, which allowed further analysis of the interfaces of those poses with a good PIPER cluster size. Five regions in the RBD were identified, which may interact with VHH 1B, 2A, and 3F, respectively (FIGS. 9A-9B). Next, 5 different S1 RBD deletion mutants were generated to validate the computationally mapped epitopes in-vitro to select the best docking model for molecular analysis. In fact, these S1 RBD deletion regions have been shown to block the S1 RBD/ACE2 interaction (Table 1).

TABLE 1
List of S1 RBD deletions and published antibodies that utilize
the deleted regions for S/ACE2 interaction blocking

| | Known antibodies utilizing |
|---|---|
| SARS-CoV-2 S1 RBD deletions | the deleted region |
| S1 RBD del 1 | S309, BD-23 |
| S1 RBD del 2 | CB6 |
| S1 RBD del 3 | B38, CB6, P2B-2F6 |
| S1 RBD del 4 | B38, CB6 P2B-2F6 |
| S1 RBD del 5 | B38, CB6 |

Wild-type and all the S1 deletion mutants were tested to assess their binding profiles with selected VHH-Fcs from Group 1 and Group 2, as well as ACE2. The binding of VHH-Fc candidates from both Group 1 and Group 2, as well as ACE2 to S1 RBD were affected following the deletion of region 1 (del1). It is possible that this result was due to a conformational change or decrease of S1 protein expression following deletion of region 1. Because based on the crystal structure of RBD/ACE2 complex (PDB 6M0J), the deleted domain is not part of the S1 RBD/ACE2 interface. Deletion of region 2 (del2) did not prevent the binding of Group 1 VHH-Fcs to S1 RBD, and in fact showed a slight increase in binding. In addition, deletion of region 2 did not prevent the binding of ACE2 to S1 RBD. However, the deletion affected the binding of Group 2 VHH-FCs to S1 RBD and it is also adjacent to a computationally predicted epitope domain in region 1. Deletion of regions 3, 4, and 5 all decreased the binding of both Group 1 and Group 2 VHH-Fcs to S1 RBD. However, these regions are more critical for Group 1 than the Group 2 for their binding. In addition, these regions are critical for ACE2 to bind to S1 RBD. Taken together, the binding epitopes for Group 1 are more associated with del3, 4 and 5 regions, whereas the epitopes for Group 2 are located closer to del2. In addition, binding variations were observed within Group 1. For example, the binding of 2A to del1, del3, del4 and del5 decreased more than that of 1B. This shows that epitopes for 2A and 1B are not the same even though they compete with each other and were initially characterized to be within the same binding Group 1 (FIGS. 9C-9D). Based on the binding and epitope binning data, 3D docking models were constructed to predict the interactions between SARS-CoV-2 S1 RBD, ACE2 and lead VHH-Fcs (FIG. 9E). Taken together, this analysis confirms that there are two major binding groups (Group 1 and Group 2) and their likely binding regions on the SARS-CoV-2 S1 protein are shown for each VHH.

### Example 6. Tri-Specific VHH-Fcs Show Potent S1 RBD Binding and S/ACE2 Blocking Activity

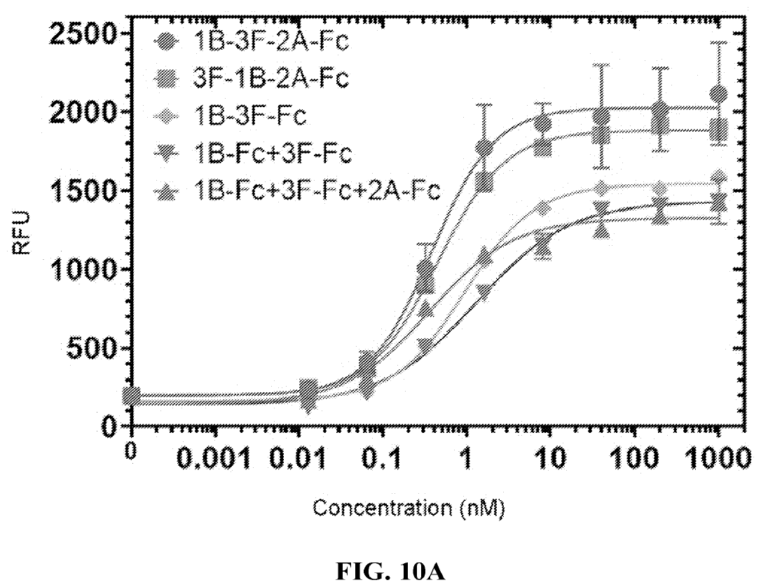

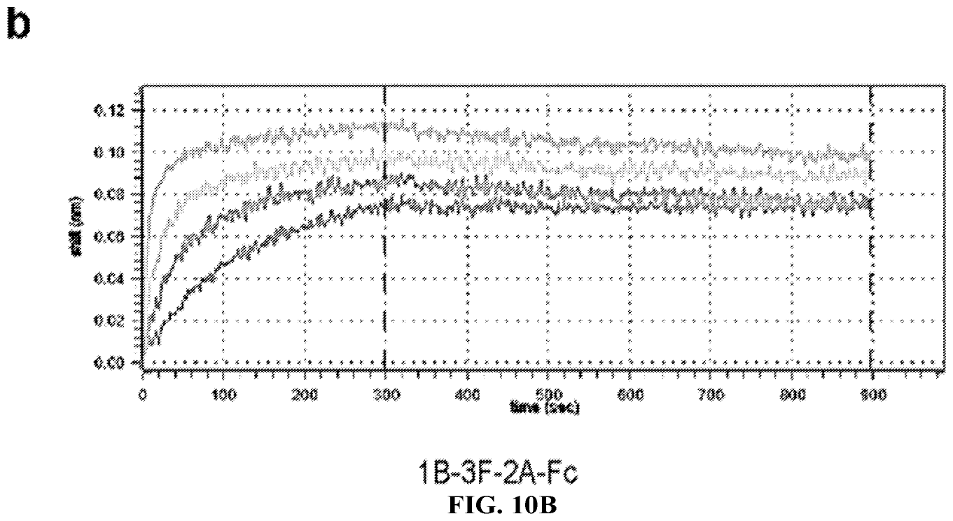

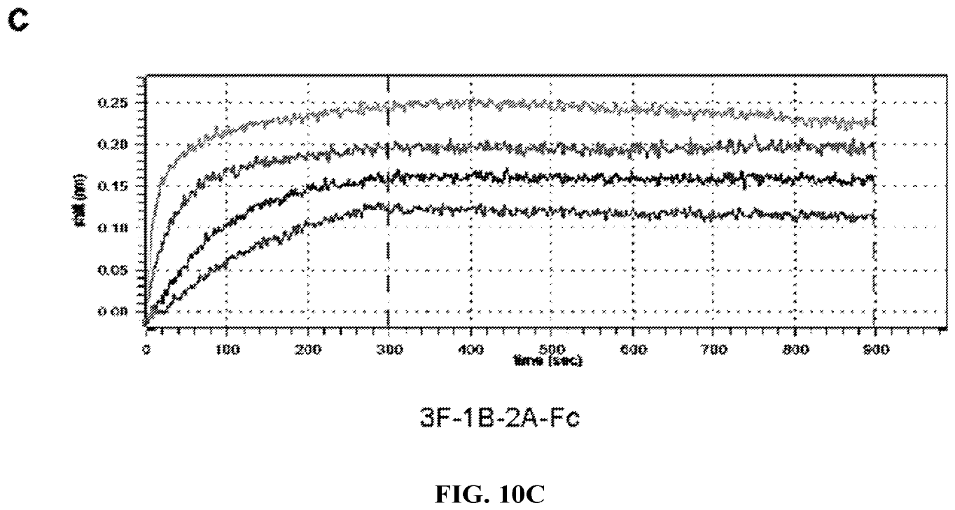

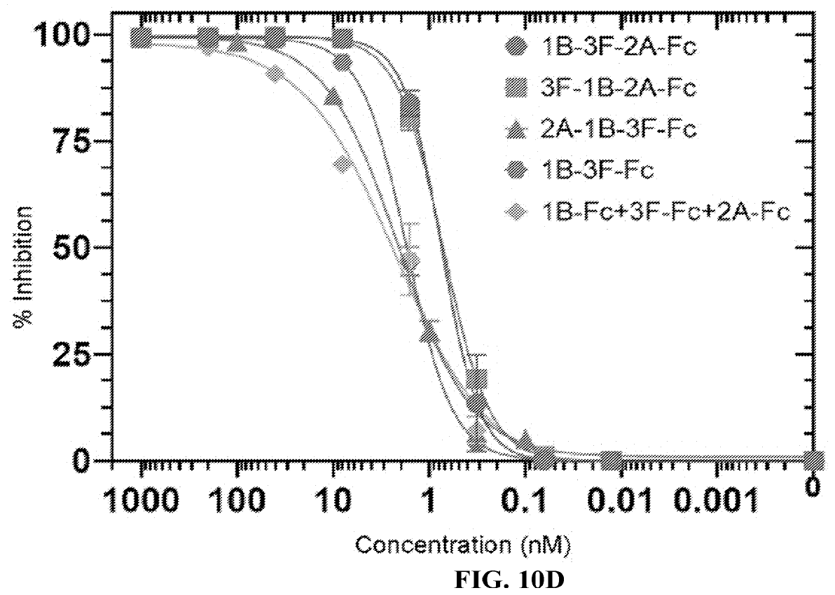

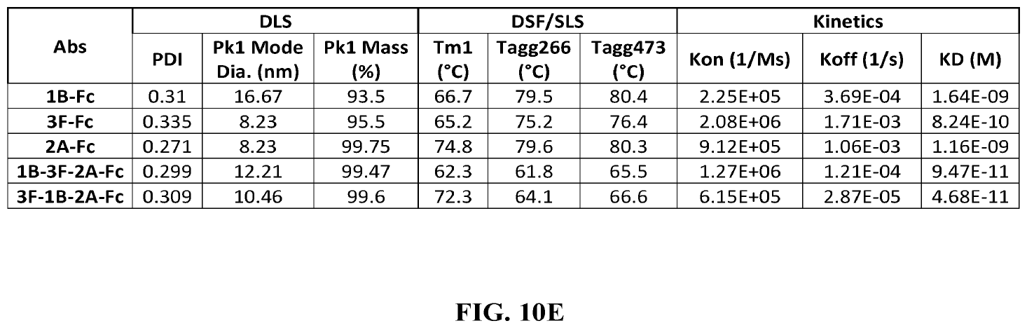

Next, whether the combination of individual VHHs binding to different S1 RBD epitopes into bi-specific antibody molecules would yield synergistic effects in SARS-CoV-2 binding and S/ACE2 blocking was tested. As expected, the resulting bi-specific VHH-Fc 1B-3F showed strong binding to S1 RBD and S/ACE2 blocking compared to individual component VHH-Fcs. Since SARS-CoV-2 S proteins form trimers, whether tri-specific antibodies with two binding units from Group 1 and another binding unit from Group 2, or vice versa, would have better binding and blocking function than the bi-specific antibody was tested. Here, only tri-specific VHH-Fcs were tested, as any larger multi-specific molecules can affect developability with Fc fusion proteins. Tri-specific VHH-Fcs were constructed. Computer-aided antibody design was used that enabled their effective construction and optimization. Then, the tri-specific, bi-specific and mono-specific VHH-Fcs were tested to determine their in vitro ability for SARS-CoV-2 S1 protein binding and S/ACE2 blocking (FIG. 10A and FIG. 10D) As expected, the multi-specific antibodies showed higher binding affinities to SARS-CoV-2 S1 protein RBD in vitro, with the tri-specific VHH-Fcs 3F-1B-2A (KD ˜0.047 nM) and 1B-3F-2A (KD ˜0.095 nM) showing more potent binding than bi-specific VHH-Fc 1B-3F (FIGS. 10A-10C, 10E). The binding affinities for tri-specific VHH-Fcs were higher than that of individual component VHH-Fcs 1B, 3F and 2A used in combination, and the binding affinity for 1B-3F-Fc was higher than that of individual component VHH-Fcs 1B, and 3F used in combination (FIG. 10A). In addition, 3F-1B-2A and 1B-3F-2A showed potent blocking of the SARS-CoV-2 S/ACE2 interaction, with IC₅₀ values of 0.71 nM and 0.74 nM, and full inhibition around 10 nM for both, respectively, which were superior to using individual component VHH-Fcs as combinations (IC₅₀ of 2.21 nM and full inhibition around 100 nM). In addition, 3F-1B-2A and 1B-3F-2A were more potent than bi-specific VHH-Fc 1B-3F in blocking SARS-CoV-2 S/ACE2 interaction (FIG. 10D). Particularly, the tri-specific VHH-Fc 2A-1B-3F exhibited a lower S/ACE2 blocking ability, indicating that the physical arrangement and/or binding orientation of the VHHs in a multi-specific antibody is important for its binding and blocking (FIG. 10D). In conclusion, this result indicates that the tri-specific VHH-Fcs have a higher synergistic potency in both binding and blocking the S1 or S1/ACE2 interaction than bi-specific or mono-specific antibodies.

### Example 7. Tri-Specific VHH-Fcs Have Favorable Developability Features

During the computer-aided design process, several development-enhancing features were incorporated in the structures of the VHH-Fcs. Therefore, the physico-chemical properties were analyzed, using DLS and DSF/SLS methods, of the lead bi- and tri-specific antibodies to determine whether they possess favorable characteristics for large-scale manufacturing that is essential for the commercial development of the antibodies (FIG. 10E). The data revealed that the lead tri-specific VHH-Fc 3F-1B-2A has lower aggregation potential based on the DLS method and is thermostable based on the DSF/SLS method (FIG. 10E).

### Example 8. Tri-Specific VHH-Fc 3F-1B-2A Neutralizes SARS-CoV-2 Infection in Cells

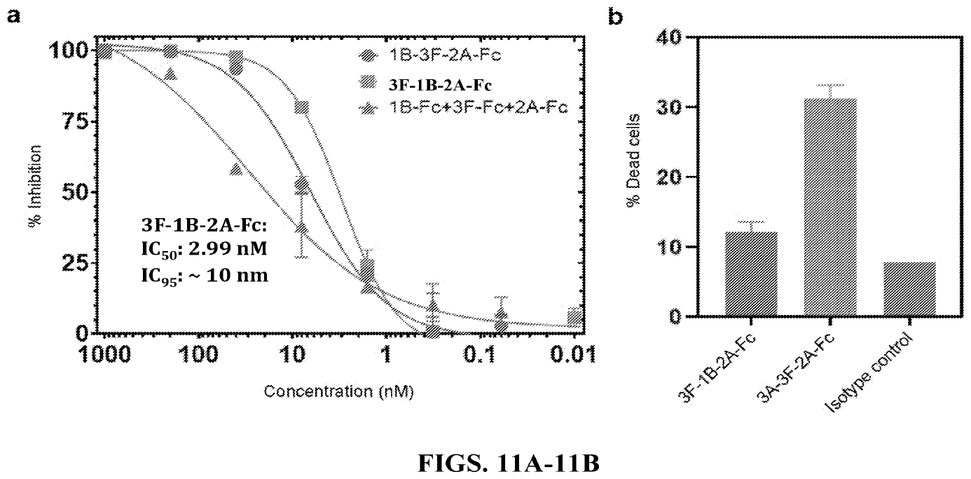

The multi-specific VHH-Fcs were tested to determine their ability to target SARS-CoV-2 in cell biological functional assays. First, the virus neutralizing ability of the antibodies were analyzed using a pseudovirus that expresses the SARS-CoV-2 S1 protein. The tri-specific VHH-Fcs 3F-1B-2A, 1B-3F-2A, and the mono-specific combinations of VHHs (1B-Fc+3F-Fc+2A-Fc) prevented the infection of human cells by the pseudoviruses (FIG. 11A). In accordance with the SARS-CoV-2 S/ACE2 blocking data, the tri-specific VHH-Fcs were more effective in neutralizing the pseudovirus infection than the combination treatment of VHH-Fcs 1B, 3F and 2A, with IC₅₀ values of 3.00 nM for 3F-1B-2A, 6.44 nM for 1B-3F-2A, and 29.19 nM for the combination treatment (FIG. 11A). This pseudovirus data confirm a synergistic effect of the tri-specific antibodies and the results suggest that the tri-specific antibodies are effective in preventing the SARS-CoV-2 infection.

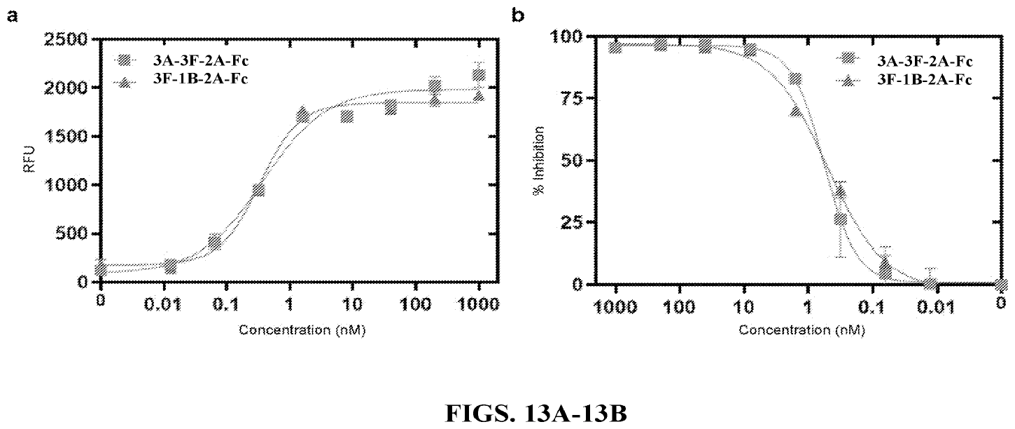

As the VHH-Fcs contain the Fc domain of human IgG1, it may be able to trigger the Fc-dependent functions to eliminate the viruses from the body. To test this, a cell line was used to transiently express the SARS-CoV-2 S1 protein. Then, the ability of the multi-specific VHH-Fcs to promote antibody-dependent cellular cytotoxicity (ADCC) was assessed, which indicates an Fc-dependent function of the antibodies. In addition to the lead tri-specific VHH-Fc antibody 3F-1B-2A-Fc, another tri-specific antibody 3A-3F-2A-Fc was constructed with similar S1 binding and S/ACE2 blocking potency (FIGS. 13A-13B). As expected, the VHH-Fcs were able to induce ADCC in the cells (FIG. 11B). This suggests that these VHH-Fcs could bind to immune cells through their Fc domain and elicit Fc-dependent functions, thereby allowing multiple mechanisms of actions against SARS-CoV-2, including binding SARS-CoV-S1 and blocking S1/ACE2 interactions.

In this study, llama-derived multi-specific nanobodies were developed and characterized that yielded data that strongly suggests their effectiveness against SARS-CoV-2 that causes COVID-19. The COVID-19 pandemic has caused widespread health and social issues around the globe, and requires therapeutics that can effectively stop and prevent the infection of SARS-CoV-2. Several monoclonal antibodies against SARS-CoV-2 have been suggested and being tested as anti-viral therapies, either as individual agents or combination therapies; however, this is the first study that introduces and demonstrates the efficacy of multi-specific antibodies against SARS-CoV-2.

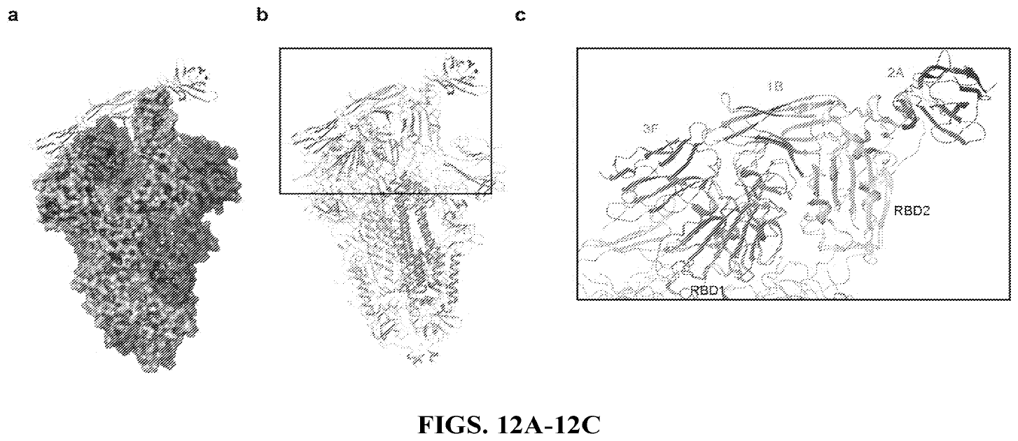

To successfully design and construct multi-specific VHH binders, the epitope information for each individual VHH clone is necessary. Here, instead of obtaining crystal structures for each antigen/antibody complex, a novel method was utilized for epitope identification. Epitope binning with Gator was performed and S/ACE2 blocking VHHs were categorized into 2 groups. The VHHs within each group competed, but there was no competition with VHHs from the other group, strongly suggesting that Group 1 and Group 2 VHHs are two separate binding groups. Then, computational VHH models were constructed and docked separately to an S1 RBD structure obtained from a publicly-available crystal structure of SARS-COV-2 S1 RBD/ACE2, and the docking structures were utilized (with higher pose cluster size) to predict possible epitopes for the individual VHHs. To validate the involvement of predicted epitopes in VHH/S1 RBD binding, the binding ability of each VHH to wild type S1 RBD, or five deletion mutants with each predicted epitope deleted, was compared. As shown in FIGS. 9C-9D, Group 1 VHHs are likely to bind to the regions del3, del4, and del5, which overlap with the ACE2 binding interface of S1 RBD. However, Group 2 VHHs are likely to bind close to the region del2, which does not overlap with ACE2 binding interface of S1 RBD. It is possible that once a region close to del2 is bound by Group 2 VHHs, it would alter the conformation of the whole RBD, consequently blocking its ability to bind to ACE2. Currently, a number of structures of S1 RBD/antibody complexes have been published. The analysis of these structures show that there are likely 2 main “hot” antibody binding regions in S1 RBD: one likely in the N-terminal region (del1), and the other likely in the ACE2 binding interface (del3, del4, del5). The selected VHH binders in tri-specific antibodies likely cover both of these regions (FIGS. 12A-12C). Based on this information, the lead tri-specific VHH-Fc format can be defined, including the linker length and the order of the VHH binders.

The tri-specific antibodies are advantageous as therapeutic agents because they simultaneously bind multiple epitopes within the S1 protein RBD that increase their antigen-binding affinity and avidity (FIGS. 12A-12C). The VHHs 1B and 3F that comprises the bi-specific antibody bind to two different epitopes in the S1 protein RBD. In the tri-specific antibody design, the VHH 2A that has an almost identical epitope as 1B was incorporated. These VHHs could bind in different orientations to the same or similar epitopes, or to a corresponding epitope in another S1 protein in the trimer, increasing the binding and blocking potency of the tri-specific VHH-Fc. In fact, this phenomenon has been previously shown for other multi-specific antibodies. For example, the CD20 targeting T cell engager antibody CD20-TCB (Roche) with two CD20 binding domains (2:1 molecular format) has increased potency compared to other CD20-binding bi-specific antibodies in clinical development (See Bacac, M. et al. CD20-TCB with Obinutuzumab Pretreatment as Next-Generation Treatment of Hematologic Malignancies. Clin Cancer Res 24, 4785-4797, doi:10.1158/1078-0432.CCR-18-0455 (2018)). In agreement with this hypothesis, the resulting tri-specific VHH-Fcs showed very potent characteristics in terms of the S binding and S/ACE2 blocking efficacy, which are among the best in currently published anti-SARS-CoV-2 therapeutic antibodies (Table 1).

Because of these characteristics, the tri-specific VHH-Fcs could be used at low concentrations for therapeutic applications that would potentially lower their toxicity in humans. In addition, the strong binding of the antibodies to the virions would minimize the risk of antibody-dependent enhancement (ADE) that is caused by sub-optimal antigen-antibody interactions and promotes enhanced viral infections. The multi-specific targeting approach also minimizes the loss of antibody binding to viral antigens due to the mutations of the viruses. The RNA viruses are known to mutate, and in this sense coronaviruses could lose the binding to antibodies relatively easily due to structural changes in the viral components. However, the VHH multi-specific antibodies would still bind to the mutated virus since the other VHHs in the tri-specific antibody would bind the unmutated epitopes of the virus. Another advantage of the VHH multi-specific platform is the ability to target multiple viruses. For example, it is possible to adjoin VHHs that bind to other coronaviruses such as SARS-CoV and MERS-CoV, and construct pan-coronavirus tri-specific VHH-Fcs that would be effective in preventing and treating a broad spectrum of coronaviruses.

The multi-specific antibody design connects human IgG1 Fc domain to bi- or tri-specific VHHs. Having the Fc domain in the antibody structure confers Fc-dependent cytotoxic functions such as ADCC, complement-dependent cytotoxicity (CDC) and antibody-dependent cellular phagocytosis (ADCP). These additional Fc-dependent functions, in addition to blocking virus entry and possible virus aggregation, would equip the VHH-Fcs with multiple mechanisms of action, making them more potent in neutralizing the coronaviruses. Indeed, the lead tri-specific VHH-Fc 3F-1B-2A show potent neutralization of SARS-CoV-2 pseudovirus infection in human cells.

One of the questions in the field of antibody therapeutics is whether the effect of using multi-specific single molecule is better than using a combination of monoclonal antibodies that collectively target the same epitopes or not. Here, it is shown that multi-specific antibodies are more effective in blocking host-virus interactions than a combination of monoclonal antibodies. The tri-specific VHH-Fc 3F-1B-A2 was much more potent in blocking the SARS-CoV-2 S/ACE2 interaction than using VHH-Fcs 3F, 1B and A2 individually as a combination. It is likely that physically combining the VHHs increases overall association constants (Kₒₙ values) and decreases overall dissociation constants (K_{off} values), producing lower binding constants, thus increasing antibody affinity towards antigens. It also increases the avidity of the antibodies, making them more effective in neutralizing viruses.

One of the hallmarks of a successful therapeutic antibody is its developability features. Especially during pandemics such as COVID-19 when rapid production of antibodies in high quantities is essential, the developability and manufacturability of the antibodies play even crucial roles. The design described herein has the advantage of using llama VHH nanobodies that have high stability. Indeed, the biochemical and biophysical characteristics of the multi-specific VHH-Fc show that they can be purified in high quantity, having better aggregation resistance, and favorable thermostability. In addition, the antibodies have high developability because the multi-specific design combines the individual VHHs into single molecules instead of combinations, making their manufacturing easier. An alternative strategy of increasing developability of the anti-SARS-CoV-2 multi-specific antibodies would be to combine 4 VHHs without the addition of IgG Fc domain to construct tetra-specific VHHs. These molecules would have the added advantage of increased affinity and avidity towards SARS-CoV-2 S1 protein compared to bi- and tri-specific VHH-Fcs, despite lacking the Fc effector functions. These tetra-specific antibodies would be ideally suited as antibody prophylactic to prevent the SARS-CoV-2 infection in humans because due to their llama VHH-only structure they would have increased thermostability, easier combination capability, and the possibility of easy large-scale manufacturing using cost-effective expression systems such as Yeast.

One of the key features of the therapeutic antibodies described herein is the use of computer-aided design that greatly reduces their development time and enhances their optimization efficiency. For instance, from the inception of this project, it was possible to produce, optimize and test the lead tri-specific VHH-Fcs in less than three months. This shows that this strategy is powerful for producing novel therapeutic antibodies for time-sensitive unmet needs, and can be utilized for future outbreaks that would require rapid development of antibody therapeutics.

### Example 9. Tri-Specific Antibody 3F-1B-2A-Fc Prevents and Treats COVID-19 Infection in Transgenic Mice Expressing Human ACE2

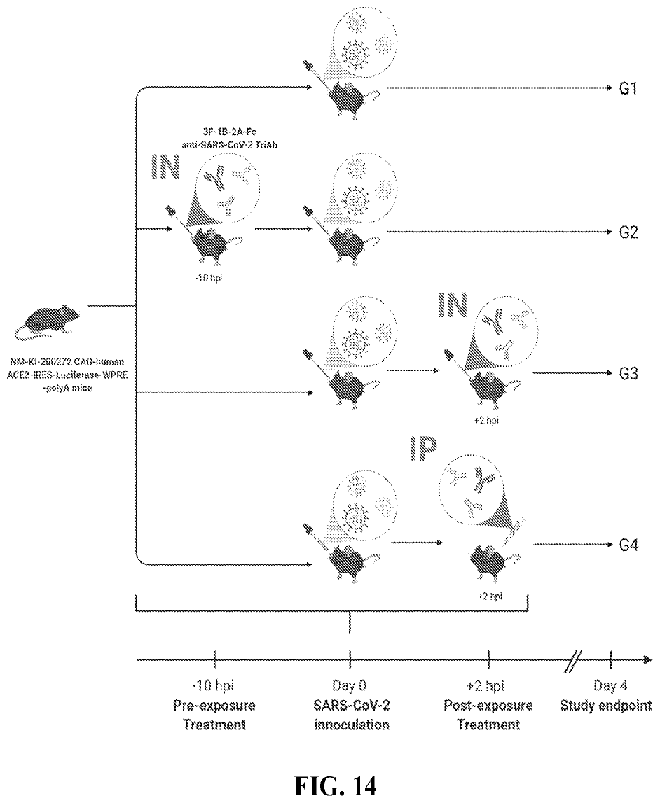

As shown in FIG. 14, NM-KI-200272 CAG-human ACE2-IRES-Luciferase-WPRE-polyA mice (adapted at Shanghai Model Organisms), were inoculated with 1000 PFU of SARS-CoV-2. The mice were then randomly placed into one control group (G1) and three treatment groups (G2-G4). All mice were inoculated with SARS-CoV-2 on Day 0 (the inoculation day) and the experiment was terminated on Day 4 (4 days post inoculation, or Study Endpoint). Specifically, in control group G1, 5 mice were inoculated with SARS-CoV-2 on Day 0, without antibody administration; in treatment group G2, 4 mice were treated with tri-specific antibody 3F-1B-3A-Fc at 25 mg/kg by intranasal (IN) administration 10 hours before the inoculation (−10 hpi), and then inoculated with SARS-CoV-2 on Day 0; in treatment group G3, 6 mice were inoculated with SARS-CoV-2 on Day 0, and then treated with 3F-1B-3A-Fc at 25 mg/kg by intranasal (IN) administration 2 hours post the infection (+2 hpi); in treatment group G4, 6 mice were inoculated with SARS-CoV-2 on Day 0, and then treated with 3F-1B-3A-Fc at 10 mg/kg by intraperitoneal (IP) administration 2 hours post the infection (+2 hpi).

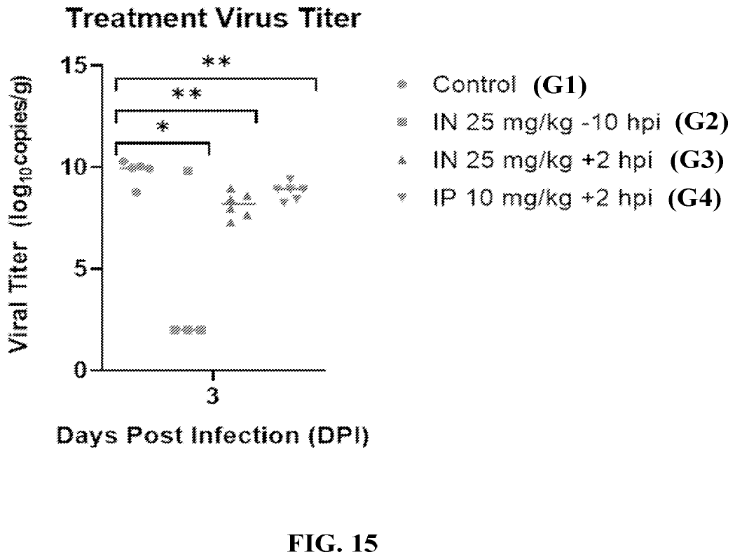

The SARS-CoV-2 viral titer in the lungs collected from the mice was determined by RT-PCR on Day 3 (3 days post inoculation). As shown in FIG. 15, both treatment strategies including administration routes (e.g., IN and IP) used in G2-G4 groups showed efficacy in reducing viral titer as compared to that of the control group (G1). In particular, the G2 group mice showed significant reduction in virus titer with undetectable levels in 3 out of the 4 mice.

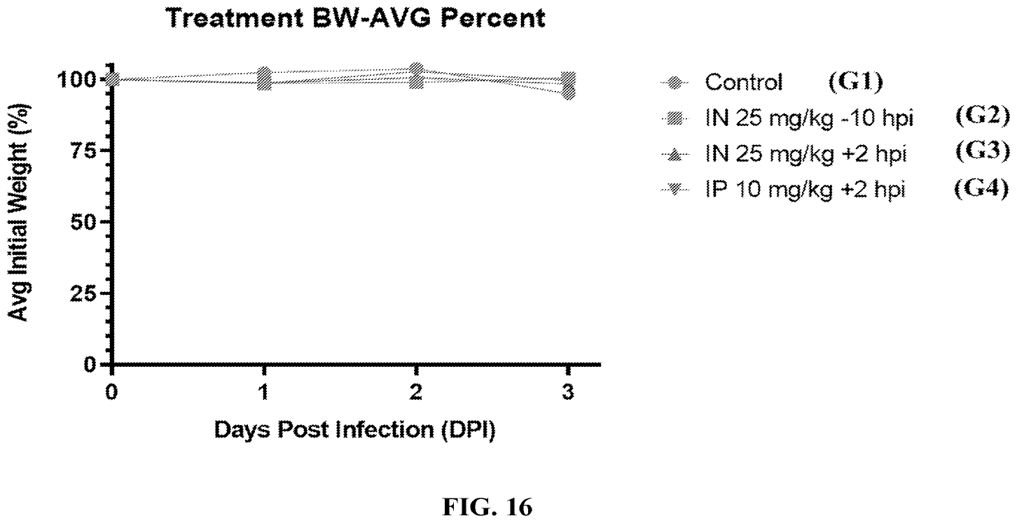

The body weight of the mice in each group was also measured daily from Day 0 to Day 3, and the result is shown in FIG. 16. During the measurement period, the body weight and survival of the treatment group mice (G2-G4) were comparable to those of the control group mice (G1), indicating that the tri-specific antibody 3F-1B-3A-Fc was well tolerated and safe to use for treating COVID-19.

### Example 10. Tri-Specific Antibody 3F-1B-2A-Fc Maintains Potent Binding and ACE2 Blocking Functions Against SARS-CoV-2 S Protein Mutants

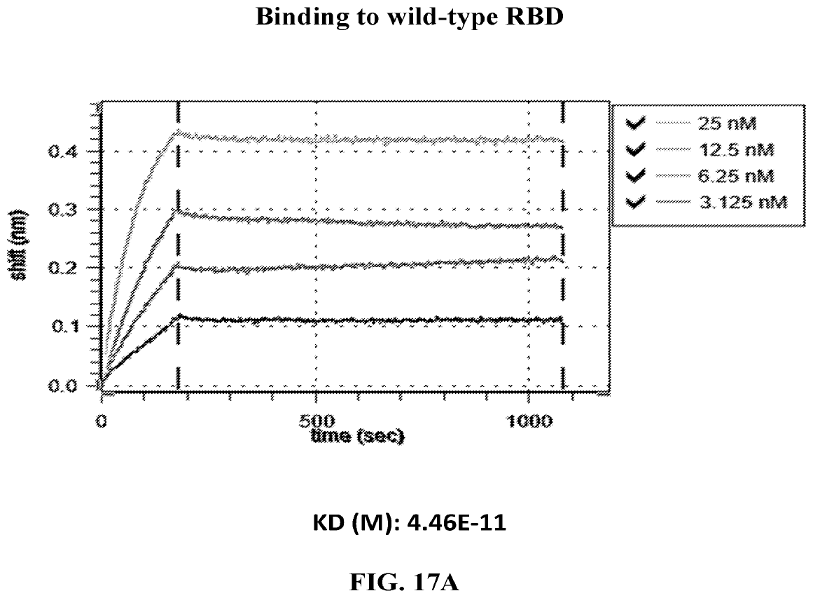

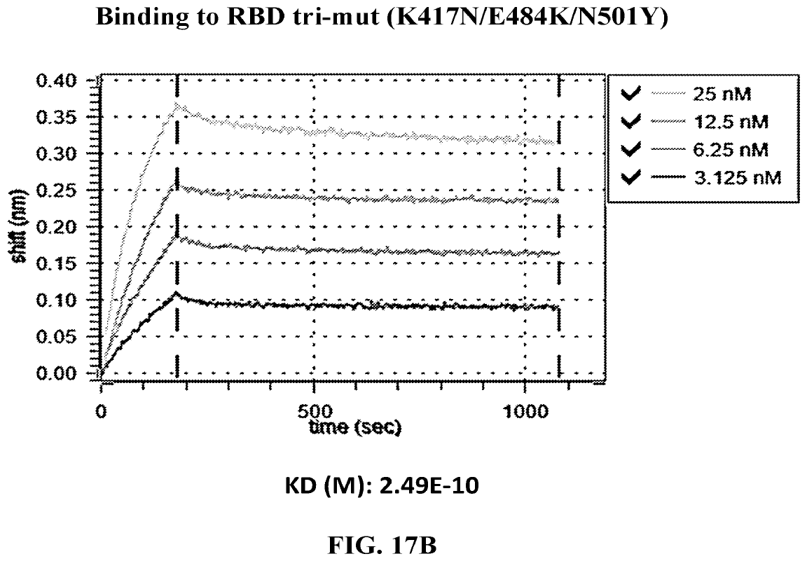

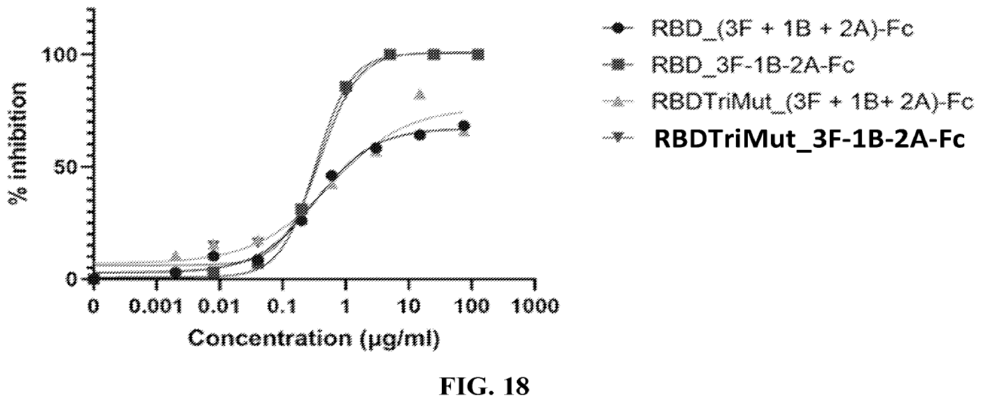

To explore whether the tri-specific antibody 3F-1B-2A-Fc can maintain binding and ACE2 blocking functions against SARS-CoV-2 S protein mutants, an S protein mutant with three mutations in the receptor binding domain (RBD), i.e., K417N, E484K, and N501Y, was purified. The S protein mutant was referred to as “tri-mut” or “RBD tri-mut”. As shown in FIGS. 17A-17B, tri-specific antibody 3F-1B-2A-Fc showed a reduced binding affinity to RBD tri-mut (KD ˜0.249 nM) relative to an S protein with wild-type RBD (KD ˜0.0446 nM). However, according to the results shown in FIG. 18, 3F-1B-2A-Fc exhibited a blocking effect to RBD tri-mut/ACE2 interaction that was comparable to that of S protein (with wild-type RBD)/ACE2 interaction. In addition, the results also showed that the blocking effect of RBD tri-mut/ACE2 interaction by tri-specific antibody 3F-1B-2A-Fc was more potent than that of individual VHH-Fcs 3F, 1B, and 2A in combination.

Other S protein mutants, e.g., D614G and N501Y, were also tested, and 3F-1B-2A-Fc showed a similar binding affinity to the mutant S proteins as compared to that of the wild-type S protein.

### Example 11. Thermostability Test of Tri-Specific Antibody 3F-1B-2A-Fc

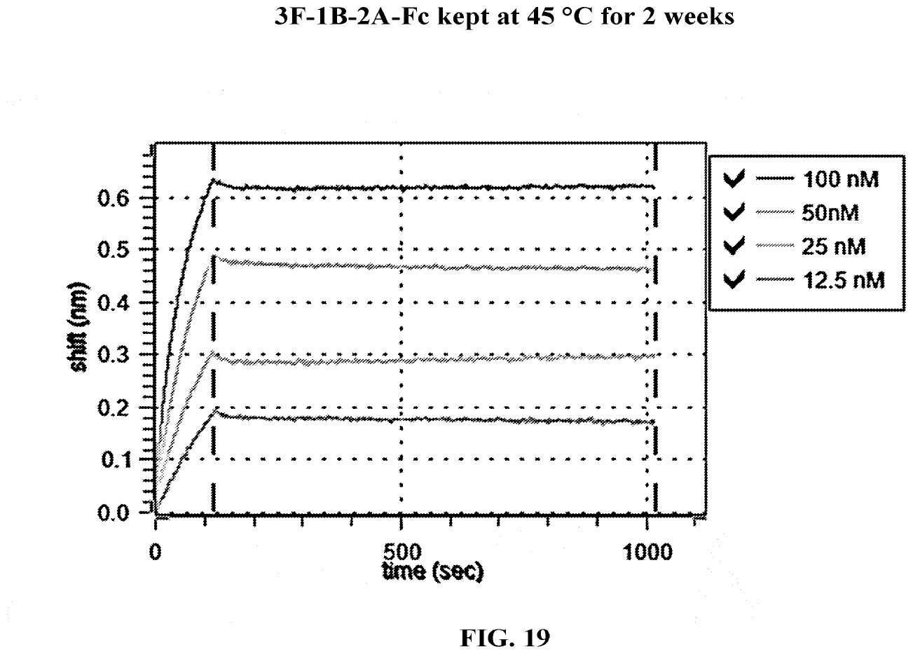

The thermostability of 3F-1B-2A-Fc was tested. Specifically, the antibody was kept at 45° C. for 2 weeks and then its binding to SARS-CoV-2 S protein was determined by bio-layer interferometry (BLI). The BLI data is shown in FIG. 19. The binding kinetic parameters Kₒₙ, K_{off}, and KD were calculated accordingly as 1.82×10⁵ /Ms, 9.90×10⁻⁵ s⁻¹, and 5.43×10⁻¹⁰ M, respectively.

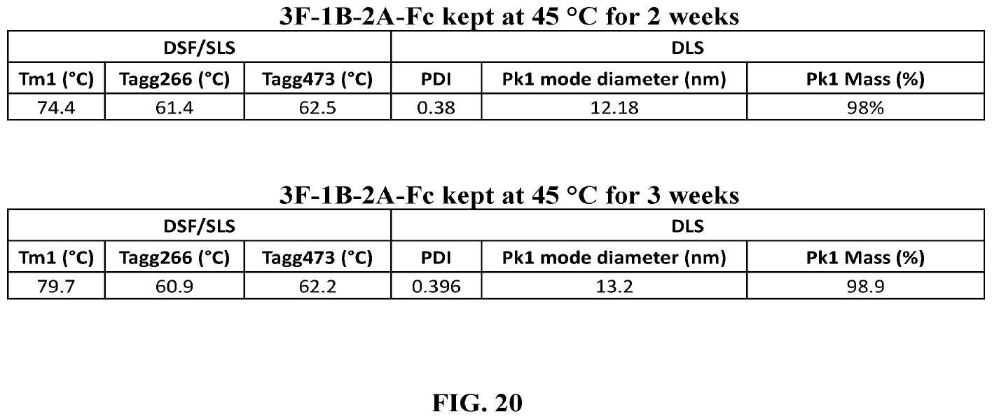

In addition, the physico-chemical properties of 3F-1B-2A-Fc was analyzed, using DLS and DSF/SLS methods, after the antibody was kept at 45° C. for 2 or 3 weeks. The results are shown in FIG. 20. The results showed that the heated 3F-1B-2A-Fc antibody had acceptable Tm and Tagg values, as compared to those determined using an un-heated antibody (See FIG. 10E).

The above results indicate that 3F-1B-2A-Fc maintains strong binding to the SARS-CoV-2 S protein after heating 3F-1B-2A-Fc at 45° C. for 2-3 weeks. The heated protein had low aggregation potential based on the DLS method and was thermostable based on the DSF/SLS method. The great thermostability allows for room temperature transportation and distribution.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to a coronavirus S protein, comprising:
a heavy chain single variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively;
(4) the selected VHH CDRs 1, 2, 3 amino acid sequences are set forth in SEQ ID NOs: 10, 11, and 12, respectively;
(5) the selected VHH CDRs 1, 2, 3 amino acid sequences are set forth in SEQ ID NOs: 13, 14, and 15, respectively;
(6) the selected VHH CDRs 1, 2, 3 amino acid sequences are set forth in SEQ ID NOs: 16, 17, and 18, respectively;
(7) the selected VHH CDRs 1, 2, 3 amino acid sequences are set forth in SEQ ID NOs: 19, 20, and 21, respectively;
(8) the selected VHH CDRs 1, 2, 3 amino acid sequences are set forth in SEQ ID NOs: 22, 23, and 24, respectively;
(9) the selected VHH CDRs 1, 2, 3 amino acid sequences are set forth in SEQ ID NOs: 25, 26, and 27, respectively;
(10) the selected VHH CDRs 1, 2, 3 amino acid sequences are set forth in SEQ ID NOs: 28, 29, and 30, respectively;
(11) the selected VHH CDRs 1, 2, 3 amino acid sequences are set forth in SEQ ID NOs: 31, 32, and 33, respectively;
(12) the selected VHH CDRs 1, 2, 3 amino acid sequences are set forth in SEQ ID NOs: 34, 35, and 36, respectively;
(13) the selected VHH CDRs 1, 2, 3 amino acid sequences are set forth in SEQ ID NOs: 37, 38, and 39, respectively;
(14) the selected VHH CDRs 1, 2, 3 amino acid sequences are set forth in SEQ ID NOs: 40, 41, and 42, respectively;
(15) the selected VHH CDRs 1, 2, 3 amino acid sequences are set forth in SEQ ID NOs: 43, 44, and 45, respectively;
(16) the selected VHH CDRs 1, 2, 3 amino acid sequences are set forth in SEQ ID NOs: 46, 47, and 48, respectively;
(17) the selected VHH CDRs 1, 2, 3 amino acid sequences are set forth in SEQ ID NOs: 49, 50, and 51, respectively;
(18) the selected VHH CDRs 1, 2, 3 amino acid sequences are set forth in SEQ ID NOs: 52, 53, and 54, respectively;
(19) the selected VHH CDRs 1, 2, 3 amino acid sequences are set forth in SEQ ID NOs: 55, 56, and 57, respectively;
(20) the selected VHH CDRs 1, 2, 3 amino acid sequences are set forth in SEQ ID NOs: 58, 59, and 60, respectively;
(21) the selected VHH CDRs 1, 2, 3 amino acid sequences are set forth in SEQ ID NOs: 61, 62, and 63, respectively;
(22) the selected VHH CDRs 1, 2, 3 amino acid sequences are set forth in SEQ ID NOs: 64, 65, and 66, respectively;
(23) the selected VHH CDRs 1, 2, 3 amino acid sequences are set forth in SEQ ID NOs: 67, 68, and 69, respectively;
(24) the selected VHH CDRs 1, 2, 3 amino acid sequences are set forth in SEQ ID NOs: 70, 71, and 72, respectively;
(25) the selected VHH CDRs 1, 2, 3 amino acid sequences are set forth in SEQ ID NOs: 73, 74, and 75, respectively;
(26) the selected VHH CDRs 1, 2, 3 amino acid sequences are set forth in SEQ ID NOs: 76, 77, and 78, respectively;
(27) the selected VHH CDRs 1, 2, 3 amino acid sequences are set forth in SEQ ID NOs: 79, 80, and 81, respectively;
(28) the selected VHH CDRs 1, 2, 3 amino acid sequences are set forth in SEQ ID NOs: 82, 83, and 84, respectively;
(29) the selected VHH CDRs 1, 2, 3 amino acid sequences are set forth in SEQ ID NOs: 85, 86, and 87, respectively;
(30) the selected VHH CDRs 1, 2, 3 amino acid sequences are set forth in SEQ ID NOs: 88, 89, and 90, respectively;
(31) the selected VHH CDRs 1, 2, 3 amino acid sequences are set forth in SEQ ID NOs: 91, 92, and 93, respectively;
(32) the selected VHH CDRs 1, 2, 3 amino acid sequences are set forth in SEQ ID NOs: 94, 95, and 96, respectively;
(33) the selected VHH CDRs 1, 2, 3 amino acid sequences are set forth in SEQ ID NOs: 97, 98, and 99, respectively;
(34) the selected VHH CDRs 1, 2, 3 amino acid sequences are set forth in SEQ ID NOs: 100, 101, and 102, respectively;
(35) the selected VHH CDRs 1, 2, 3 amino acid sequences are set forth in SEQ ID NOs: 103, 104, and 105, respectively;
(36) the selected VHH CDRs 1, 2, 3 amino acid sequences are set forth in SEQ ID NOs: 106, 107, and 108, respectively;
(37) the selected VHH CDRs 1, 2, 3 amino acid sequences are set forth in SEQ ID NOs: 109, 110, and 111, respectively;
(38) the selected VHH CDRs 1, 2, 3 amino acid sequences are set forth in SEQ ID NOs: 112, 113, and 114, respectively;
(39) the selected VHH CDRs 1, 2, 3 amino acid sequences are set forth in SEQ ID NOs: 115, 116, and 117, respectively;
(40) the selected VHH CDRs 1, 2, 3 amino acid sequences are set forth in SEQ ID NOs: 118, 119, and 120, respectively;
(41) the selected VHH CDRs 1, 2, 3 amino acid sequences are set forth in SEQ ID NOs: 121, 122, and 123, respectively;
(42) the selected VHH CDRs 1, 2, 3 amino acid sequences are set forth in SEQ ID NOs: 124, 125, and 126, respectively;
(43) the selected VHH CDRs 1, 2, 3 amino acid sequences are set forth in SEQ ID NOs: 127, 128, and 129, respectively;
(44) the selected VHH CDRs 1, 2, 3 amino acid sequences are set forth in SEQ ID NOs: 130, 131, and 132, respectively;
(45) the selected VHH CDRs 1, 2, 3 amino acid sequences are set forth in SEQ ID NOs: 133, 134, and 135, respectively;
(46) the selected VHH CDRs 1, 2, 3 amino acid sequences are set forth in SEQ ID NOs: 136, 137, and 138, respectively;
(47) the selected VHH CDRs 1, 2, 3 amino acid sequences are set forth in SEQ ID NOs: 139, 140, and 141, respectively;
(48) the selected VHH CDRs 1, 2, 3 amino acid sequences are set forth in SEQ ID NOs: 142, 143, and 144, respectively;
(49) the selected VHH CDRs 1, 2, 3 amino acid sequences are set forth in SEQ ID NOs: 145, 146, and 147, respectively;
(50) the selected VHH CDRs 1, 2, 3 amino acid sequences are set forth in SEQ ID NOs: 148, 149, and 150, respectively;
(51) the selected VHH CDRs 1, 2, 3 amino acid sequences are set forth in SEQ ID NOs: 151, 152, and 153, respectively;
(52) the selected VHH CDRs 1, 2, 3 amino acid sequences are set forth in SEQ ID NOs: 154, 155, and 156, respectively;
(53) the selected VHH CDRs 1, 2, 3 amino acid sequences are set forth in SEQ ID NOs: 157, 158, and 159, respectively;
(54) the selected VHH CDRs 1, 2, 3 amino acid sequences are set forth in SEQ ID NOs: 160, 161, and 162, respectively;
(55) the selected VHH CDRs 1, 2, 3 amino acid sequences are set forth in SEQ ID NOs: 163, 164, and 165, respectively;
(56) the selected VHH CDRs 1, 2, 3 amino acid sequences are set forth in SEQ ID NOs: 166, 167, and 168, respectively;
(57) the selected VHH CDRs 1, 2, 3 amino acid sequences are set forth in SEQ ID NOs: 169, 170, and 171, respectively;
(58) the selected VHH CDRs 1, 2, 3 amino acid sequences are set forth in SEQ ID NOs: 172, 173, and 174, respectively;
(59) the selected VHH CDRs 1, 2, 3 amino acid sequences are set forth in SEQ ID NOs: 175, 176, and 177, respectively;
(60) the selected VHH CDRs 1, 2, 3 amino acid sequences are set forth in SEQ ID NOs: 178, 179, and 180, respectively;
(61) the selected VHH CDRs 1, 2, 3 amino acid sequences are set forth in SEQ ID NOs: 181, 182, and 183, respectively;
(62) the selected VHH CDRs 1, 2, 3 amino acid sequences are set forth in SEQ ID NOs: 184, 185, and 186, respectively;
(63) the selected VHH CDRs 1, 2, 3 amino acid sequences are set forth in SEQ ID NOs: 187, 188, and 189, respectively;
(64) the selected VHH CDRs 1, 2, 3 amino acid sequences are set forth in SEQ ID NOs: 190, 191, and 192, respectively;
(65) the selected VHH CDRs 1, 2, 3 amino acid sequences are set forth in SEQ ID NOs: 193, 194, and 195, respectively;
(66) the selected VHH CDRs 1, 2, 3 amino acid sequences are set forth in SEQ ID NOs: 196, 197, and 198, respectively;
(67) the selected VHH CDRs 1, 2, 3 amino acid sequences are set forth in SEQ ID NOs: 199, 200, and 201, respectively;
(68) the selected VHH CDRs 1, 2, 3 amino acid sequences are set forth in SEQ ID NOs: 202, 203, and 204, respectively;
(69) the selected VHH CDRs 1, 2, 3 amino acid sequences are set forth in SEQ ID NOs: 205, 206, and 207, respectively;
(70) the selected VHH CDRs 1, 2, 3 amino acid sequences are set forth in SEQ ID NOs: 208, 209, and 210, respectively;
(71) the selected VHH CDRs 1, 2, 3 amino acid sequences are set forth in SEQ ID NOs: 211, 212, and 213, respectively;
(72) the selected VHH CDRs 1, 2, 3 amino acid sequences are set forth in SEQ ID NOs: 214, 215, and 216, respectively;
(73) the selected VHH CDRs 1, 2, 3 amino acid sequences are set forth in SEQ ID NOs: 217, 218, and 219, respectively;
(74) the selected VHH CDRs 1, 2, 3 amino acid sequences are set forth in SEQ ID NOs: 220, 221, and 222, respectively;
(75) the selected VHH CDRs 1, 2, 3 amino acid sequences are set forth in SEQ ID NOs: 223, 224, and 225, respectively;
(76) the selected VHH CDRs 1, 2, 3 amino acid sequences are set forth in SEQ ID NOs: 226, 227, and 228, respectively;
(77) the selected VHH CDRs 1, 2, 3 amino acid sequences are set forth in SEQ ID NOs: 229, 230, and 231, respectively;
(78) the selected VHH CDRs 1, 2, 3 amino acid sequences are set forth in SEQ ID NOs: 232, 233, and 234, respectively;
(79) the selected VHH CDRs 1, 2, 3 amino acid sequences are set forth in SEQ ID NOs: 235, 236, and 237, respectively;
(80) the selected VHH CDRs 1, 2, 3 amino acid sequences are set forth in SEQ ID NOs: 238, 239, and 240, respectively;
(81) the selected VHH CDRs 1, 2, 3 amino acid sequences are set forth in SEQ ID NOs: 241, 242, and 243, respectively;
(82) the selected VHH CDRs 1, 2, 3 amino acid sequences are set forth in SEQ ID NOs: 244, 245, and 246, respectively;
(83) the selected VHH CDRs 1, 2, 3 amino acid sequences are set forth in SEQ ID NOs: 247, 248, and 249, respectively;
(84) the selected VHH CDRs 1, 2, 3 amino acid sequences are set forth in SEQ ID NOs: 250, 251, and 252, respectively;
(85) the selected VHH CDRs 1, 2, 3 amino acid sequences are set forth in SEQ ID NOs: 253, 254, and 255, respectively;
(86) the selected VHH CDRs 1, 2, 3 amino acid sequences are set forth in SEQ ID NOs: 256, 257, and 258, respectively; and
(87) the selected VHH CDRs 1, 2, 3 amino acid sequences are set forth in SEQ ID NOs: 259, 260, and 261, respectively.

2. The antibody or antigen-binding fragment thereof of claim 1, wherein the VHH comprises CDRs 1, 2, 3 with the amino acid sequences set forth in SEQ ID NOs: 1, 2, and 3, respectively.

3. An antibody or antigen-binding fragment thereof that binds to a coronavirus S protein comprising a heavy chain single variable region (VHH) comprising an amino acid sequence that is at least 80% identical to a selected VHH sequence, wherein the selected VHH sequence is selected from the group consisting of SEQ ID NOS: 262-348.

4. The antibody or antigen-binding fragment thereof of claim 3, wherein the VHH comprises the sequence of SEQ ID NO: 262.

5. The antibody or antigen-binding fragment thereof of any one of claims 1-4, wherein the antibody or antigen-binding fragment specifically binds to a coronavirus S protein.

6. The antibody or antigen-binding fragment thereof of any one of claims 1-5, wherein the antibody or antigen-binding fragment is a humanized antibody or antigen-binding fragment thereof

7. An antibody or antigen-binding fragment thereof comprising the VHH CDRs 1, 2, 3, of the antibody or antigen-binding fragment thereof of any one of claims 1-6.

8. The antibody or antigen-binding fragment thereof of any one of claims 1-7, wherein the antibody or antigen-binding fragment comprises a human IgG Fc.

9. The antibody or antigen-binding fragment thereof of any one of claims 1-8, wherein the antibody or antigen-binding fragment comprises two or more heavy chain single variable domains.

10. A nucleic acid comprising a polynucleotide encoding the antibody or antigen-binding fragment thereof any one of claims 1-9.

11. The nucleic acid of claim 10, wherein the nucleic acid is cDNA.

12. A vector comprising one or more of the nucleic acids of any one of claims 10-11.

13. A cell comprising the vector of claim 12.

14. The cell of claim 13, wherein the cell is a CHO cell.

15. A cell comprising one or more of the nucleic acids of claims 10 or 11.

16. A method of producing an antibody or an antigen-binding fragment thereof, the method comprising
(a) culturing the cell of any one of claims 13-15 under conditions sufficient for the cell to produce the antibody or the antigen-binding fragment; and
(b) collecting the antibody or the antigen-binding fragment produced by the cell.

17. A method of treating a subject having a coronavirus-related disease, the method comprising administering a therapeutically effective amount of a composition comprising the antibody or antigen-binding fragment thereof of any one of claims 1-9 to the subject.

18. A method of neutralizing a coronavirus, the method comprising
contacting the coronavirus with an effective amount of a composition comprising
an antibody or antigen-binding fragment thereof of any one of claims 1-9.

19. A method of blocking internalization of a coronavirus by a cell, the method comprising
contacting the coronavirus with an effective amount of a composition comprising
the antibody or antigen-binding fragment thereof of any one of claims 1-9.

20. A method of identifying a subject as having a coronavirus disease, the method comprising
detecting a sample collected from the subject as having the coronavirus by the antibody or antigen-binding fragment thereof of any one of claims 1-9,
thereby identifying the subject as having a coronavirus infection.

21. The method of claim 20, wherein the sample is a blood sample, a saliva sample, a stool sample, or a liquid sample from the respiratory tract of the subject.

22. The method of any one of claims 17-21, wherein the coronavirus is SARS-CoV-2.

23. The method of any one of claims 17-21, wherein the coronavirus is MERS-CoV.

24. The method of any one of claims 17-21, wherein the coronavirus is SARS-CoV.

25. A pharmaceutical composition comprising the antibody or antigen-binding fragment thereof of any one of claims 1-9, and a pharmaceutically acceptable carrier.

26. An antibody or antigen-binding fragment thereof that cross-competes with the antibody or antigen-binding fragment thereof of any one of claims 1-9.
